SEQUENCE LISTING (37 sequences)

SEQ ID NO: 1 - DNA, 9401 bp, Hepatitis C virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcttgactgtgcccgcttcggcctaccaagtgcgcaactccacggggctttaccacgtcaccaatgattgccctaactcgagtattgtgtacgaggcggccgatgccatcctgcacactccggggtgcgtcccttgcgttcgtgagggcaacgcctcgaggtgttgggtggcgatgacccctacggtggccaccagggatggcaaactccccgcgacgcagcttcgacgtcacatcgatctgcttgtcgggagcgccaccctctgttcggccctctacgtgggggacctatgcgggtctgtctttcttgtcggccaactgttcaccttctctcccaggcgccactggacgacgcaaggttgcaattgctctatctatcccggccatataacgggtcaccgcatggcatgggatatgatgatgaactggtcccctacgacggcgttggtaatggctcagctgctccggatcccacaagccatcttggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccacactgtgtctggatttgttagcctcctcgcaccaggcgccaagcagaacgtccagctgatcaacaccaacggcagttggcacctcaatagcacggccctgaactgcaatgatagcctcaacaccggctggttggcagggcttttctatcaccacaagttcaactcttcaggctgtcctgagaggctagccagctgccgaccccttaccgattttgaccagggctggggccctatcagttatgccaacggaagcggccccgaccagcgcccctactgctggcactaccccccaaaaccttgcggtattgtgcccgcgaagagtgtgtgtggtccggtatattgcttcactcccagccccgtggtggtgggaacgaccgacaggtcgggcgcgcccacctacagctggggtgaaaatgatacggacgtcttcgtccttaacaataccaggccaccgctgggcaattggttcggttgtacctggatgaactcaactggattcaccaaagtgtgcggagcgcctccttgtgtcatcggaggggcgggcaacaacaccctgcactgccccactgattgcttccgcaagcatccggacgccacatactctcggtgcggctccggtccctggatcacacccaggtgcctggtcgactacccgtataggctttggcattatccttgtaccatcaactacaccatatttaaaatcaggatgtacgtgggaggggtcgaacacaggctggaagctgcctgcaactggacgcggggcgaacgttgcgatctggaagacagggacaggtccgagctcagcccgttactgctgaccactacacagtggcaggtcctcccgtgttccttcacaaccctaccagccttgtccaccggcctcatccacctccaccagaacattgtggacgtgcagtacttgtacggggtggggtcaagcatcgcgtcctgggccattaagtgggagtacgtcgttctcctgttccttctgcttgcagacgcgcgcgtctgctcctgcttgtggatgatgctactcatatcccaagcggaggcggctttggagaacctcgtaatacttaatgcagcatccctggccgggacgcacggtcttgtatccttcctcgtgttcttctgctttgcatggtatttgaagggtaagtgggtgcccggagcggtctacaccttctacgggatgtggcctctcctcctgctcctgttggcgttgccccagcgggcgtacgcgctggacacggaggtggccgcgtcgtgtggcggtgttgttctcgtcgggttgatggcgctgactctgtcaccatattacaagcgctatatcagctggtgcttgtggtggcttcagtattttctgaccagagtggaagcgcaactgcacgtgtggattccccccctcaacgtccgaggggggcgcgacgccgtcatcttactcatgtgtgctgtacacccgactctggtatttgacatcaccaaattgctgctggccgtcttcggacccctttggattcttcaagccagtttgcttaaagtaccctactttgtgcgcgtccaaggccttctccggttctgcgcgttagcgcggaagatgatcggaggccattacgtgcaaatggtcatcattaagttaggggcgcttactggcacctatgtttataaccatctcactcctcttcgggactgggcgcacaacggcttgcgagatctggccgtggctgtagagccagtcgtcttctcccaaatggagaccaagctcatcacgtggggggcagataccgccgcgtgcggtgacatcatcaacggcttgcctgtttccgcccgcaggggccgggagatactgctcgggccagccgatggaatggtctccaaggggtggaggttgctggcgcccatcacggcgtacgcccagcagacaaggggcctcctagggtgcataatcaccagcctaactggccgggacaaaaaccaagtggagggtgaggtccagattgtgtcaactgctgcccaaaccttcctggcaacgtgcatcaatggggtgtgctggactgtctaccacggggccggaacgaggaccatcgcgtcacccaagggtcctgtcatccagatgtataccaatgtagaccaagaccttgtgggctggcccgctccgcaaggtagccgctcattgacaccctgcacttgcggctcctcggacctttacctggtcacgaggcacgccgatgtcattcccgtgcgccggcggggtgatagcaggggcagcctgctgtcgccccggcccatttcctacttgaaaggctcctcggggggtccgctgttgtgccccgcggggcacgccgtgggcatatttagggccgcggtgtgcacccgtggagtggctaaggcggtggactttatccctgtggagaacctagagacaaccatgaggtccccggtgttcacggataactcctctccaccagtagtgccccagagcttccaggtggctcacctccatgctcccacaggcagcggcaaaagcaccaaggtcccggctgcatatgcagctcagggctataaggtgctagtactcaacccctctgttgctgcaacactgggctttggtgcttacatgtccaaggctcatgggatcgatcctaacatcaggaccggggtgagaacaattaccactggcagccccatcacgtactccacctacggcaagttccttgccgacggcgggtgctcggggggcgcttatgacataataatttgtgacgagtgccactccacggatgccacatccatcttgggcatcggcactgtccttgaccaagcagagactgcgggggcgagactggttgtgctcgccaccgccacccctccgggctccgtcactgtgccccatcccaacatcgaggaggttgctctgtccaccaccggagagatccctttttacggcaaggctatccccctcgaagtaatcaagggggggagacatctcatcttctgtcattcaaagaagaagtgcgacgaactcgccgcaaagctggtcgcattgggcatcaatgccgtggcctactaccgcggtcttgacgtgtccgtcatcccgaccagcggcgatgttgtcgtcgtggcaaccgatgccctcatgaccggctataccggcgacttcgactcggtgatagactgcaatacgtgtgtcacccagacagtcgatttcagccttgaccctaccttcaccattgagacaatcacgctcccccaggatgctgtctcccgcactcaacgtcggggcaggactggcagggggaagccaggcatctacagatttgtggcaccgggggagcgcccctccggcatgttcgactcgtccgtcctctgtgagtgctatgacgcaggctgtgcttggtatgagctcacgcccgccgagactacagttaggctacgagcgtacatgaacaccccggggcttcccgtgtgccaggaccatcttgaattttgggagggcgtctttacaggcctcactcatatagatgcccactttctatcccagacaaagcagagtggggagaaccttccttacctggtagcgtaccaagccaccgtgtgcgctagggctcaagcccctcccccatcgtgggaccagatgtggaagtgtttgattcgcctcaagcccaccctccatgggccaacacccctgctatacagactgggcgctgttcagaatgaaatcaccctgacgcacccagtcaccaaatacatcatgacatgcatgtcggccgacctggaggtcgtcacgagcacctgggtgctcgttggcggcgtcctggctgctttggccgcgtattgcctgtcaacaggctgcgtggtcatagtgggcagggtcgtcttgtccgggaagccggcaatcatacctgacagggaagtcctctaccgagagttcgatgagatggaagagtgctctcagcacttaccgtacatcgagcaagggatgatgctcgccgagcagttcaagcagaaggccctcggcctcctgcagaccgcgtcccgtcaggcagaggttatcgcccctgctgtccagaccaactggcaaaaactcgagaccttctgggcgaagcatatgtggaacttcatcagtgggatacaatacttggcgggcttgtcaacgctgcctggtaaccccgccattgcttcattgatggcttttacagctgctgtcaccagcccactaaccactagccaaaccctcctcttcaacatattgggggggtgggtggctgcccagctcgccgcccccggtgccgctactgcctttgtgggcgctggcttagctggcgccgccatcggcagtgttggactggggaaggtcctcatagacatccttgcagggtatggcgcgggcgtggcgggagctcttgtggcattcaagatcatgagcggtgaggtcccctccacggaggacctggtcaatctactgcccgccatcctctcgcccggagccctcgtagtcggcgtggtctgtgcagcaatactgcgccggcacgttggcccgggcgagggggcagtgcagtggatgaaccggctgatagccttcgcctcccgggggaaccatgtttcccccacgcactacgtgccggagagcgatgcagctgcccgcgtcactgccatactcagcagcctcactgtaacccagctcctgaggcgactgcaccagtggataagctcggagtgtaccactccatgctccggttcctggctaagggacatctgggactggatatgcgaggtgttgagcgactttaagacctggctaaaagctaagctcatgccacagctgcctgggatcccctttgtgtcctgccagcgcgggtataagggggtctggcgagtggacggcatcatgcacactcgctgccactgtggagctgagatcactggacatgtcaaaaacgggacgatgaggatcgtcggtcctaggacctgcaggaacatgtggagtgggaccttccccattaatgcctacaccacgggcccctgtaccccccttcctgcgccgaactacacgttcgcgctatggagggtgtctgcagaggaatatgtggagataaggcaggtgggggacttccactacgtgacgggtatgactactgacaatctcaaatgcccgtgccaggtcccatcgcccgaatttttcacagaattggacggggtgcgcctacataggtttgcgcccccctgcaagcccttgctgcgggaggaggtatcattcagagtaggactccacgaatacccggtagggtcgcaattaccttgcgagcccgaaccggacgtggccgtgttgacgtccatgctcactgatccctcccatataacagcagaggcggccgggcgaaggttggcgaggggatcacccccctctgtggccagctcctcggctagccagctatccgctccatctctcaaggcaacttgcaccgctaaccatgactcccctgatgctgagctcatagaggccaacctcctatggaggcaggagatgggcggcaacatcaccagggttgagtcagaaaacaaagtggtgattctggactccttcgatccgcttgtggcggaggaggacgagcgggagatctccgtacccgcagaaatcctgcggaagtctcggagattcgcccaggccctgcccgtttgggcgcggccggactataaccccccgctagtggagacgtggaaaaagcccgactacgaaccacctgtggtccatggctgtccgcttccacctccaaagtcccctcctgtgcctccgcctcggaagaagcggacggtggtcctcactgaatcaaccctatctactgccttggccgagctcgccaccagaagctttggcagctcctcaacttccggcattacgggcgacaatacgacaacatcctctgagcccgccccttctggctgcccccccgactccgacgctgagtcctattcctccatgccccccctggagggggagcctggggatccggatcttagcgacgggtcatggtcaacggtcagtagtgaggccaacgcggaggatgtcgtgtgctgctcaatgtcttactcttggacaggcgcactcgtcaccccgtgcgccgcggaagaacagaaactgcccatcaatgcactaagcaactcgttgctacgtcaccacaatttggtgtattccaccacctcacgcagtgcttgccaaaggcagaagaaagtcacatttgacagactgcaagttctggacagccattaccaggacgtactcaaggaggttaaagcagcggcgtcaaaagtgaaggctaacttgctatccgtagaggaagcttgcagcctgacgcccccacactcagccaaatccaagtttggttatggggcaaaagacgtccgttgccatgccagaaaggccgtaacccacatcaactccgtgtggaaagaccttctggaagacaatgtaacaccaatagacactaccatcatggctaagaacgaggttttctgcgttcagcctgagaaggggggtcgtaagccagctcgtctcatcgtgttccccgatctgggcgtgcgcgtgtgcgaaaagatggctttgtacgacgtggttacaaagctccccttggccgtgatgggaagctcctacggattccaatactcaccaggacagcgggttgaattcctcgtgcaagcgtggaagtccaagaaaaccccaatggggttctcgtatgatacccgctgctttgactccacagtcactgagagcgacatccgtacggaggaggcaatctaccaatgttgtgacctcgacccccaagcccgcgtggccatcaagtccctcaccgagaggctttatgttgggggccctcttaccaattcaaggggggagaactgcggctatcgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagcgcgggggtccaggaggacgcggcgagcctgagagccttcacggaggctatgaccaggtactccgccccccctggggaccccccacaaccagaatacgacttggagctcataacatcatgctcctccaacgtgtcagtcgcccacgacggcgctggaaagagggtctactacctcacccgtgaccctacaacccccctcgcgagagctgcgtgggagacagcaagacacactccagtcaattcctggctaggcaacataatcatgtttgcccccacactgtgggcgaggatgatactgatgacccatttctttagcgtccttatagccagggaccagcttgaacaggccctcgattgcgagatctacggggcctgctactccatagaaccacttgatctacctccaatcattcaaagactccatggcctcagcgcattttcactccacagttactctccaggtgaaattaatagggtggccgcatgcctcagaaaacttggggtaccgcccttgcgagcttggagacaccgggcccggagcgtccgcgctaggcttctggccagaggaggcagggctgccatatgtggcaagtacctcttcaactgggcagtaagaacaaagctcaaactcactccaatagcggccgctggccagctggacttgtccggctggttcacggctggctacagcgggggagacatttatcacagcgtgtctcatgcccggccccgctggatctggttttgcctactcctgcttgctgcaggggtaggcatctacctcctccccaaccgatgaaggttggggtaaacactccggcct

SEQ ID NO: 2 - protein, 3011 aa, Hepatitis C virus
MSTNPKPQKKNKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNASRCWVAMTPTVATRDGKLPATQLRRHIDLLVGSATLCSALYVGDLCGSVFLVGQLFTFSPRRHWTTQGCNCSIYPGHITGHRMAWDMMMNWSPTTALVMAQLLRIPQAILDMIAGAHWGVLAGIAYFSMVGNWAKVLVVLLLFAGVDAETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGKWVPGAVYTFYGMWPLLLLLLALPQRAYALDTEVAASCGGVVLVGLMALTLSPYYKRYISWCLWWLQYFLTRVEAQLHVWIPPLNVRGGRDAVILLMCAVHPTLVFDITKLLLAVFGPLWILQASLLKVPYFVRVQGLLRFCALARKMIGGHYVQMVIIKLGALTGTYVYNHLTPLRDWAHNGLRDLAVAVEPVVFSQMETKLITWGADTAACGDIINGLPVSARRGREILLGPADGMVSKGWRLLAPITAYAQQTRGLLGCIITSLTGRDKNQVEGEVQIVSTAAQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVIAPAVQTNWQKLETFWAKHMWNFISGIQYLAGLSTLPGNPAIASLMAFTAAVTSPLTTSQTLLFNILGGWVAAQLAAPGAATAFVGAGLAGAAIGSVGLGKVLIDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYKGVWRVDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNR

SEQ ID NO: 3 - protein, 9 aa, Influenza A virus
AKSFYKNLI

SEQ ID NO: 4 - protein, 9 aa, Influenza A virus
KGNSYPKLS

SEQ ID NO: 5 - protein, 9 aa, Influenza A virus
QQSLYQNAD

SEQ ID NO: 6 - protein, 9 aa, Influenza A virus
NADAYVFVG

SEQ ID NO: 7 - protein, 9 aa, Influenza A virus
VDGWYGYHH

SEQ ID NO: 8 - protein, 9 aa, Influenza A virus
VKNLYEKVR

SEQ ID NO: 9 - protein, 9 aa, Influenza A virus
KNGTYDYPK

SEQ ID NO: 10 - protein, 9 aa, Influenza A virus
STRIYQILA

SEQ ID NO: 11 - protein, 9 aa, Influenza A virus
ILAIYSTVA

SEQ ID NO: 12 - protein, 9 aa, Influenza A virus
DVPDYASLR

SEQ ID NO: 13 - protein, 9 aa, Influenza A virus
LNFKYPALN

SEQ ID NO: 14 - protein, 9 aa, Influenza A virus
FDKLYIWGV

SEQ ID NO: 15 - protein, 9 aa, Influenza A virus
MDGWYGFRH

SEQ ID NO: 16 - protein, 9 aa, Influenza A virus
DVPDYASLR

SEQ ID NO: 17 - protein, 9 aa, Influenza A virus
LNFKYPALN

SEQ ID NO: 18 - protein, 9 aa, Influenza A virus
FDKLYIWGV

SEQ ID NO: 19 - protein, 9 aa, Influenza A virus
VDGWYGFRH

SEQ ID NO: 20 - protein, 9 aa, Influenza A virus
DHDVYRDEA

SEQ ID NO: 21 - protein, 9 aa, Influenza B virus
LHEKYGGLN

SEQ ID NO: 22 - protein, 9 aa, Human immunodeficiency virus type 1
WVTVYYGVP

SEQ ID NO: 23 - protein, 9 aa, Human immunodeficiency virus type 1
SNDTYRLIN

SEQ ID NO: 24 - protein, 9 aa, Human immunodeficiency virus type 1
IRQAYCNLS

SEQ ID NO: 25 - protein, 9 aa, Human immunodeficiency virus type 1
GEFFYCNTT

SEQ ID NO: 26 - protein, 9 aa, Human immunodeficiency virus type 1
LNNTYTKEK

SEQ ID NO: 27 - protein, 9 aa, Human immunodeficiency virus type 1
SNKTYDTIW

SEQ ID NO: 28 - protein, 9 aa, Human immunodeficiency virus type 1
EIDNYTNII

SEQ ID NO: 29 - protein, 9 aa, Human immunodeficiency virus type 1
TNIIYSLIE

SEQ ID NO: 30 - protein, 9 aa, Human immunodeficiency virus type 1
VRQGYSPLS

SEQ ID NO: 31 - protein, 9 aa, Human immunodeficiency virus type 1
WVTVYYGVP

SEQ ID NO: 32 - protein, 9 aa, Human immunodeficiency virus type 1
SSEYYRLIN

SEQ ID NO: 33 - protein, 9 aa, Human immunodeficiency virus type 1
GQTFYATGD

SEQ ID NO: 34 - protein, 9 aa, Human immunodeficiency virus type 1
GEFFYCNTS

SEQ ID NO: 35 - protein, 9 aa, Human immunodeficiency virus type 1
FNGTYNWTE

SEQ ID NO: 36 - protein, 9 aa, Human immunodeficiency virus type 1
TNTIYRLLE

SEQ ID NO: 37 - protein, 9 aa, Human immunodeficiency virus type 1
VRQGYSPLS

(19) United States
(12) Patent Application Publication
(10) Pub. No.: US 2016/0067331 A1
(43) Pub. Date: Mar. 10, 2016

(54) VIRAL PROTEINS AS IMMUNOMODULATORY AGENTS AND VACCINE COMPONENTS

(71) Applicant: THE UNIVERSITY OF IOWA RESEACH FOUNDATION, Iowa City, IA (US)

(72) Inventors: Jack T. STAPLETON, Iowa City, IA (US); Nirjal BHATTARAI, Coralville, IA (US); Jinhua XIANG, Iowa City, IA (US); James H. MCLINDEN, Coralville, IA (US)

(21) Appl. No.: 14/773,855

(22) PCT Filed: Mar. 14, 2014
(86) PCT No.: PCT/US14/27147

§ 371 Date: Sep. 9, 2015

Related U.S. Application Data
(60) Provisional application No. 61/787,895, filed on Mar. 15, 2013.

(51) Int. Cl. A61K 39/29; A61K 39/21; A61K 39/145; A61K 39/12; A61K 45/06; C12N 7/00

(52) U.S. Cl. CPC A61K 39/29; A61K 45/06; C12N 7/00; A61K 39/145; A61K 39/12; A61K 39/21; A61K 2039/55

(57) ABSTRACT

The invention provides compositions and methods involving viral envelope polypeptides and peptides for use in modulating immune responses, including inhibition inflammation related to pathogenic T-cell activation. In addition, modification of the viral sequences responsible for modulating immune response provides for improved vaccine formulations.

This application claims benefit of priority to U.S. Provisional Application Ser. No. 61/787,895, filed Mar. 15, 2013, the entire contents of which are hereby incorporated by reference.

This invention was made with government support under Grant No. RO1 AI-58740 awarded by the National Institutes of Allergy and Infectious Disease and Merit Review Grant I01BX000207 from the Department of Veterans Affairs. The United States Government has certain rights in the invention.

### BACKGROUND OF THE INVENTION

I. Field of the Invention

The present invention relates generally to the fields of molecular biology and virology. More particularly, it concerns methods and compositions to treat inflammatory conditions, in particular those resulting from pathologic T-cell activation. It also relates to improved vaccine formulations.

II. Description of Related Art

GB virus C (GBV-C) is a human virus within the Flaviviridae that is related to hepatitis C virus (HCV) (Stapleton et al., 2011). Although GBV-C infection is common and about 2% of healthy U.S. blood donors have viremia at the time of donation, it is not associated with any disease (Mohr et al., 2010; Stapleton et al., 2011). Due to shared routes of transmission, the rate of GBV-C infection is high among HIV-infected individuals, with a prevalence of up to 42% (Mohr et al., 2010; Rey et al., 2000). GBV-C is lymphotropic, and virus particles are produced when lymphocytes from infected subjects are cultured ex vivo (George et al., 2006; Rydze et al., 2012). Several clinical studies, including a meta-analysis of HIV-positive subjects found an association between persistent GBV-C infection and prolonged survival in HIV-infected individuals (Nunnari et al., 2003; Tillmann et al., 2001; Williams et al., 2004; Xiang et al., 2001; Zhang et al., 2006). Although several mechanisms have been proposed for this beneficial association between GBV-C coinfection and HIV-related survival (Bhattarai and Stapleton, 2012), recent studies suggest that GBV-C reduces HIV-associated chronic immune activation, and that this contributes to better HIV clinical outcomes (Bhattarai et al., 2012a; Bhattarai et al., 2012b; Maidana-Giret et al., 2009; Rydze et al., 2012; Schwarze-Zander et al., 2010; Stapleton et al., 2012; Stapleton et al., 2009).

HIV infection is associated with chronic immunoactivation that contributes to HIV mediated immune dysfunction, and immune activation facilitates HIV replication and pathogenesis (Grossman et al., 2006; Hazenberg et al., 2003). Although combination antiretroviral therapy (cART) suppresses HIV plasma viral load (VL), the level of immune activation markers do not return to levels observed in HIV-uninfected individuals (Hunt et al., 2008; Vinikoor et al., 2013). In addition, persistent immune activation observed in HIV-treated individuals is associated with a reduced response to HIV therapy (Deeks et al., 2004; Hunt et al., 2003). Among HIV-infected subjects GBVC coinfection is associated reduced immune activation independent of HIV VL or cART (Bhattarai et al., 2012b; Maidana-Giret et al., 2009; Stapleton et al., 2012), suggesting that GBV-C infection alters immune activation pathways. Since GBV-C replication in vitro is reduced by T cell activation (Rydze et al., 2012), the development of mechanisms to inhibit immune activation is beneficial for the virus. Understanding mechanisms by which GBV-C reduces chronic immune activation in HIV-infected subjects may lead to novel approaches to treat HIV infection and HIV associated chronic immune activation. Indeed, by interfering with T cell activation pathways, many viruses increase the likelihood that it will cause persistent infection. Furthermore, by interfering with antigen presentation this impairs the ability to elicit memory T and B cell responses or high titers of antibodies.

### SUMMARY OF THE INVENTION

Thus, in accordance with the present invention, there is provided a method of inhibiting immune cell activation comprising administering to a mammalian subject in need thereof an RNA virus envelope peptide or polypeptide comprising an immunomodulatory domain. In particular, the RNA virus envelope peptide polypeptide is not GBV-C E2. The peptide or polypeptide may comprise about 16, 17, 18, 19, 20, 21, 22, 23, 24, 25, 30, 35, 40, 45, 50, 75, 100, 150, 175, 200, 219, 250 consecutive residues of a native envelope polypeptide or immunomodulatory domain. The peptide or polypeptide may comprise HCV E2 sequences, and may further comprise non-HCV E2 sequences. The immune cell may be a T cell or a B cell. The T cell may be a helper T cell suppressor T cell, or a killer T cell. The subject is a human or a non-human mammal Administering may comprise intravenous, intraarterial, oral, subcutaneous, topical or intraperitoneal administration.

The method may further comprise administering a second anti-inflammatory agent. The second anti-inflammatory agent may a steroid or a COX-2 inhibitor, may be contacted prior to said peptide or polypeptide, after said peptide or polypeptide or at the same time as said peptide or polypeptide. The peptide or polypeptide may comprise all L amino acids, all D amino acids, or a mix of L and D amino acids. The peptide or polypeptide may be administered at 0.1-500 mg/kg/d. The peptide or polypeptide may be administered daily or weekly. The peptide or polypeptide may be administered daily for 7 days, 2 weeks, 3 weeks, 4 weeks, one month, 6 weeks, 8 weeks, two months, 12 weeks, or 3 months, or weekly for 2 weeks, 3 weeks, 4 weeks, 6 weeks, 8 weeks, 10 weeks, or 12 weeks. The peptide or polypeptide is derived from Hepatitis C Virus E2, Hepatitus E Virus, Human Immunodeficiency Virus envelope gp120/160, Yellow Fever Virus envelope protein, Bovine Viral Diarrhea Virus envelope protein, Classical Swine Fever Virus envelope protein, influenza envelope protein, Dengue Virus envelope protein, West Nile Virus envelope protein, and Japanese Encephalitis Virus envelope protein.

Also provided is a composition comprising a peptide or polypeptide comprising a peptide segment as shown in FIG. 19 or 21, formulated with a pharmaceutically acceptable carrier buffer or diluent. The peptide or polypeptide may comprise about 10, 11, 12, 13, 14, 15, 16, 17, 18, 19, 20, 21, 22, 23, 24, 25, 30, 35, 40, 45, 50, 75, or 100 consecutive residues of the native polypeptide from which it is derived. The peptide or polypeptide may be about 10, 11, 12, 13, 14, 15, 16, 17, 18, 19, 20, 21, 22, 23, 24, 25, 30, 35, 40, 45, 50, 75, 100, 125, 150, 175, 200, 219 or 250 residues in length. The peptide or polypeptide may be fused to a non-viral sequence. The composition may be formulated for pharmaceutical administration, such as topical, cutaneous, subcutaneous, alimentrary or parenteral administration.

In another embodiment, there is provided a method of inducing an immune response in an mammalian subject comprising administering to said subject with an RNA virus envelope protein wherein said envelope protein comprises one or more modified kinase sites. The modified kinase site may comprise a deleted kinase site or a mutated kinase site. The RNA virus may be from the Reoviridae, Atroviridae, Caliciviridae, Hepeviridae, Picornaviridae, Togaviridae, Flaviviridae, Coronaviridae, Orthomyxoviridae, Arenaviridae, Bunyaviridae, Paramyxoviridae, Filoviridae, Rabdoviridae, or Retroviridae family. RNA virus is GBV-C, Hepatitis C Virus, Hepatitis E Virus, Human Immunodeficiency Virus, influenza virus, Dengue Virus, West Nile Virus, Japanese Encephalitis Virus, Bovine Viral Diarrhea Virus, Classical Swine Fever Virus or Yellow Fever Virus. The subject may be a human or non-human mammal.

The envelope protein may be free from other viral components. The envelope protein may be comprised in a subunit vaccine comprising other viral components but lacking intact virions. The enveloped protein may be comprised in a killed whole virion. The enveloped protein may be comprised in a live attenuated virus. The envelope protein may be administered with a second envelope protein from a distinct serotype or strain of said virus. The envelope protein may be administered more than once. The envelope protein may be formulated with an adjuvant. The envelope protein may comprise a modification to a site shown in Table 5 or FIG. 19 or 21. The envelope protein may comprise a modification to a site shown in FIG. 20. The modified kinase site is an Lck site or Fyn site. The envelope protein may be GBV-C E2, such as where a proline of a PXXP motif, wherein X is any amino acid, at GBV-C E2 positions 48, 51, 257 or 260, is changed to another amino acid.

Also provided is a vaccine comprising an RNA virus envelope protein having a modification in a peptide segment shown in Table 5 or FIG. 19 or 21. The modification may comprise a deleted segment or a mutated segment. The peptide or polypeptide may comprise about 10, 11, 12, 13, 14, 15, 16, 17, 18, 19, 20, 21, 22, 23, 24, 25, 30, 35, 40, 45, 50, 75, or 100 consecutive residues of the native polypeptide from which it is derived. The peptide or polypeptide may be about 10, 11, 12, 13, 14, 15, 16, 17, 18, 19, 20, 21, 22, 23, 24, 25, 30, 35, 40, 45, 50, 75, 100, 125, 150, 175, 200, 219 or 250 residues in length. The peptide or polypeptide may be fused to a non-viral sequence. The vaccine may be formulated with an adjuvant. The envelope protein is GBV-C E2, such as where a proline of a PXXP motif, wherein X is any amino acid, at GBV-C E2 positions 48, 51, 257 or 260 is changed to another amino acid.

The use of the word “a” or “an” when used in conjunction with the term “comprising” in the claims and/or the specification may mean “one,” but it is also consistent with the meaning of “one or more,” “at least one,” and “one or more than one.” Furthermore, where multiple steps of a method of process are cited, it is understood that the steps are not required to be performed in the particular order recited unless one of skill in the art is not be able to practice the method in a different order.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F. Extracellular microvesicles from GBV-C infected human serum inhibit T cell receptor (TCR) signaling in primary human T cells. GBV-C RNA concentration in peripheral blood mononuclear cells (PBMC), and in purified CD4+ and CD8+ T cells obtained from nine GBV-C-infected subjects (FIG. 1A). GBV-C RNA concentration in serum, the extracellular microvesicle (EMV) pellet or supernatant purified from the serum of five GBV-C-infected individuals (FIG. 1B). GBV-C RNA concentration in the top and bottom fraction of serum separated using saline flotation gradient centrifugation (FIG. 1C). IL-2 release by PBMCs (FIG. 1D) and CD69 and CD25 cell surface expression (FIG. 1E, FIG. 1F) of PBMCs incubated with GBV-C positive (GB+) or negative (GB−) serum-derived EMV following activation with CD3 and CD28 antibodies. −Fold change was calculated using CD69 and CD25 MFI levels before and after stimulation. MFI=mean fluorescence intensity. Data represent the average of three independent cultures, independently performed three times. *P<0.05; **P<0.01.

FIGS. 2A-D. GBV-C E2 protein expression inhibits T cell receptor (TCR) mediated activation of CD4+ T cells. Jurkat (tet-off) cells stably expressed GBV-C E2 protein or the same GBV-C sequence with a plus one frame shift to abolish translation (FS). Incubation with doxycycline (dox; 1 μg/ml) for 5 days significantly reduced E2 protein expression (FIG. 2A). Twenty four hours after TCR stimulation with CD3 and CD28 antibodies, CD69 surface expression was significantly reduced in Jurkat cells expressing E2 protein, and this was reversed by maintaining cells in doxycycline (FIG. 2B). Data represent the −fold increase in CD69 expression before and after TCR stimulation from three independent cultures. *P<0.05; **P<0.001. Phosphorylation of the linker for activation of T cells (LAT; FIG. 2C) and the zeta-chain-associated protein kinase (ZAP)-70 (FIG. 2D) in GBV-C E2 expressing Jurkat cells compared to the frameshift control (FS) with TCR activation using anti-CD3. MFI=mean fluorescence intensity. Each experiment was repeated at least three times with consistent results.

FIGS. 3A-D. GBV-C E2 protein interacts with and inhibits Lck activation. Phosphorylation of Lck Y394 in Jurkat cells expressing GBV-C E2 protein compared to the frameshift (FS) control following TCR stimulation with anti-CD3 by immunoblot (FIG. 3A). The data were quantified by densitometry (FIG. 3B). Following precipitation with protein A/G, Jurkat cell lysates incubated with recombinant GBV-C E2-human Fc fusion (E2-Fc) also precipitated Lck but not Zap-70 or LAT, whereas addition of IgG to the lysates did not precipitate Lck (FIG. 3C). Similarly, anti-Lck precipitation of Jurkat cells expressing GBV-C E2 protein with precipitated E2 protein, but did not precipitate nonspecific IgG (FIG. 3D). Each experiment was repeated at least three times with consistent results.

FIGS. 4A-E. Characterization of a peptide domain within GBV-C E2 that inhibits T cell receptor (TCR) signaling. FIG. 4A illustrates Jurkat cells lines generated that stably expressed GBV-C E2 proteins (amino acid numbers shown). *=previously described cell lines. Cell lines that did not inhibit TCR signaling are shaded (FIG. 4B). IL-2 release following TCR stimulation with anti-CD3/CD28 is shown for cell lines expressing various E2 amino acids (FIG. 4D). −Fold change in IL-2 release was calculated by measuring IL-2 at baseline (˜5 pg/ml) and after anti-CD3/CD28 stimulation for 24 hours. Recombinant E2 protein was phosphorylated by Lck in an in vitro kinase reaction (FIG. 4E), and was dephosphorylated by the CD45 phosphatase (FIG. 4C). Data represents average from three independent cultures, independently performed three times. Each experiment was repeated at least three times with consistent results. *P<0.01.

FIGS. 5A-D. Synthetic GBV-C E2 peptides inhibit TCR activation in primary human T cells. IL-2 release (FIG. 5A) from primary human PBMCs, and cell surface CD69 and CD25 expression on primary human CD4+ (FIG. 5B) and CD8+ (FIG. 5C) T cells following TCR-stimulation with anti-CD3/CD28. Cells were incubated in synthetic peptides with an N-terminal HIV TAT protein transduction domain including the native GBV-C E2 86-101 sequence (TAT-Y87), the same sequence with a histidine substitution for Y87 (TAT-Y87H), the TAT sequence alone (TAT-only), or with no peptide. Lck-mediated phosphorylation of synthetic peptides including the GBV-C amino acid 276-292 sequence (TAT-276-292), the TAT-Y87 peptide synthetically phosphorylated (TAT-Y87PO4) or the 276-292 peptide amino acids synthesized in a scrambled order TAT-SCR (FIG. 5D). RLU=relative luminescence units. Each experiment was repeated at least three times with consistent results. *P<0.05, **P<0.01.

FIGS. 6A-G. GBV-C E2 protein inhibits T cell receptor (TCR) signaling in bystander cells. IL-2 release (FIG. 6A) by, and surface expression of CD69 (FIG. 6B) and CD25 (FIG. 6C) on Jurkat cells (JC; GFP negative) following TCR stimulation with anti-CD3/CD28. Cells were cocultured with GBV-C E2 expressing cells (GFP positive) or with vector control cells (VC; GFP positive). Detection of GBV-C E2 protein and CD63 in extracellular microvescles (EMV) purified from cell culture supernatants (see methods) of Jurkat cells expressing GBV-C E2 protein or the frameshift control (FS) (FIG. 6D). Following TCR stimulation with anti-CD3/CD28, IL-2 release (FIG. 6E), CD69 and CD25 cell surface expression (FIG. 6F, FIG. 6G) in PBMCs obtained from healthy, GBV-C negative donors. Cells were incubated with GBV-C E2 positive extracellular microvesicles (E2 EMV) or GBV-C negative microvesicles (FS EMV). Fold change was calculated by measuring IL-2, CD69 and CD25 levels before and after stimulation. US=unstimulated, MFI=mean fluorescence intensity. Data represent the average of three independent cultures. *P<0.01, **P<0.01.

FIG. 7. Sorting strategy for CD4+ and CD8+ T cell purification from GBV-C viremic subjects. CD3+ T cells were enriched using magnetic bead immunoaffinity selection followed by flow cytometric (FACS) purification of CD4+ and CD8+ T cells. CD4+ and CD8+ T cell purity was greater than 99%.

FIGS. 8A-B. GBV-C E2 protein expression reduces LAT and ZAP-70 phosphorylation. Phosphorylation of LAT (Y191) was significantly inhibited following TCR activation in Jurkat cells expressing GBV-C E2 protein compared to the frameshift control (FS) as determined by ELISA (FIG. 8A). ELISA data represent the average LAT phosphorylation from three independent cultures. Fold change in phosphorylation of ZAP-70 (Y319) following TCR activation was measured by densitometry of immune blot (FIG. 8B). Each experiment was repeated at least three times with consistent results. *P<0.05; **P<0.01.

FIGS. 9A-C. GBV-C E2 protein does not alter CD45 and Csk expression. Expression of CD45 (FIGS. 9A-B) and Csk (C) was not different in GBV-C E2 expressing cells compared to the FS control. Recombinant GBV-C E2 protein did not reduce CD45 enzymatic function (FIG. 9C). NS=not significant. Each experiment was repeated twice with consistent results.

FIG. 10. GFP expression by Jurkat cell lines. Jurkat cell lines expressing human GBV-C E2 protein truncated mutants and E2 protein from chimpanzee GBV-C (GBV-Ccpz) isolate stably expressed GFP as determined by flow cytometry.

FIGS. 11A-D. Sequence alignment of E2 protein from human and chimpanzee GBV-C isolates. GBV-C E2 protein sequences from human GBV-C (GBV-Chum) and chimpanzee GBV-C (GBV-Ccpz) isolates for the two predicted Lck substrate motifs (aa 83-91) (FIG. 11A) and (aa 281-289) (FIG. 11B), and the two SH3 binding motifs (aa 48-51) (FIG. 11C) and (aa 257-260) (FIG. 11D). The GenBank accession numbers for isolates (top to bottom) include: U36380, AB003291, AB013500, AF104403, AB003289, AB013501, AF031827, AF031828, AF031829, AF081782, D90600, AF121950, AF309966, AY196904, D87255, U63715, NC001710, U44402, U45966, U94695, AB003288, AB003290, AB003293, AB008335, AB008342, AF006500, D87262, D87263, D87708, D87709, D87710, D87711, D87712, D87713, D87714, D87715, D90601, U75356, AB021287, AB018667, AY949771, AB003292, K7117, DH028, D1185, AF070476, JX472278.1, JX472279.1.

FIGS. 12A-D. Uptake of TAT-fused peptides in PBMCs. Flowcytometric analysis of PBMCs following 24 hour incubation with FITC-labelled synthetic peptides. No peptide control (FIG. 12A), control peptides with an HIV TAT protein transduction domain sequence at the N-terminus (TAT-only) (FIG. 12B), peptides representing GBV-C aa 86-98 (Y87) with TAT (FIG. 12C), and the same sequence with a histidine substitution for tyrosine (Y87H) (FIG. 12D). Each experiment was conducted in triplicate and repeated on a separate day with consistent results.

FIGS. 13A-C. Viral protein expression inhibiting IL-2 release. (FIG. 13A) HCV E2 protein is expressed in Jurkat cells, but not vector control (VC). (FIG. 13B) IL-2 release from unstimulated Jurkat cells (US), or Jurkat cells containing the VC, HCV E2, GBV-C E2, YFV envelope or the chimpanzee variant of GBV-C E2. (FIG. 13C) Recombinant HCV E2 protein was phosphorylated by Lck in vitro, and was dephosphorylated by CD45 in vitro.

FIG. 14. HCV E2 protein inhibits activation of Lck, and downstream signaling molecules ZAP-70 and LAT following TCR engagement. Following anti-CD3 antibody engagement of the T cell receptor, activation of Lck (phosphorylation of Y394), ZAP-70 (phosphorylation of Y319), and LAT (phospohorylation of Y226) was significantly greater in vector control Jurkat cells compared to Jurkat cells expressing HCV E2 protein. Baseline (0) and time in minutes following application of anti-CD3 antibody is shown (2, 5, 15).

FIGS. 15A-B. HCV E2 Protein inhibits Jurkat cell activation by either anti-CD3/CD28 or PMA-ionomycin. IL-2 release by Jurkat cells expressing either the vector control or HCV E2 protein (both GFP positive from vector) was measured following stimulation with anti-CD3/CD28 (FIG. 15A) or PMA-ionomycin (FIG. 15B).

FIG. 16. HCV E2 inhibits upregulation of activation markers CD69 and CD25 by anti-CD3/CD28, but not PMA-ionomycin. HCV E2 expressing Jurkat cells or control Jurkat cells were stimulated anti-CD3/CD28 and the activation markers CD69 and CD25 were measured on the cell surfaces by flow cytometry (top four panels). HCV E2 blocked upregulation of these markers compared to controls. In contrast, CD69 and CD25 upregulation were not different in HCV E2 expressing and control Jurkat cells stimulated with PMA-ionomycin (bottom four panels).

FIG. 17. T cell activation pathways through the T cell receptor, PMA and ionomycin.

FIG. 18. Predicted PK specific kinase sites. Predictions are all of the predicted kinase targets (using a web-based PK-specific phosphorylation site prediction program) within an E2 predicted amino acid sequence generated from the nucleotide sequence of an Iowa HCV isolate. This HCV isolate was determined to be genotype 1a by commercial testing methods. As can be seen, using Lck as a model, there are only two Lck sites in this sequence. Nevertheless, numerous additional signaling pathways may be inhibited which may contribute to the poor immunogenicity of these proteins.

FIG. 19. Predicted Lck substrate sequences within the HCV E2 proteins from different HCV isolates. In contrast to the isolate shown in FIG. 18, multiple (up to five in a given isolate) Lck sites are predicted in other HCV E2 sequences. Based on alignments, the Lck sites listed at amino acid position 124 and 211 are highly conserved, and thus are the most likely to be operative. However, if additional Lck sites influence T cell responses, they may well contribute to altered pathogenicity.

FIG. 20. Predicted kinase motifs in the E2 coding region of BVDV 1 and 2 isolates.

FIG. 21. YFV replication increased in cells lacking LCK. YFV but not mumps replication was significantly greater in JCaM cells (that lack Lck) compared to JCaM cells in which Lck was restored (JCaM/Lck). **=p<0.01; NS=not significant.

FIGS. 22A-B. TCR-activation inhibits YFV. YFV replication was reduced in Jurkat cells (with Lck) activated with anti-CD3 for 24 hrs prior to YFV infection (FIG. 22A), but not in Jurkat cells lacking Lck. YFV RNA measured 5 days post infection. In contrast, no difference in YFV replication was noted in cells infected with YFV for 4 days prior to 24 hrs activation with anti-CD3 in Lck+ and Lck− cells (FIG. 22B), As before, replication was significantly greater in Lck− cells compared to Lck+ cells.

FIGS. 23A-B. Lck inhibitor enhances YFV replication. Incubation of Jurkat cells (FIG. 23A) or primary human CD3+ T cells (FIG. 23B) in the Lck inhibitor II (pyrrolo[2,3-d]pyrimidines containing a 5-[4-phenoxyphenyl]) significantly enhanced YFV replication over 5 days in culture. **=p<0.01 and *=p<0.05 compared to no inhibitor.

FIGS. 24A-B. YFV particles inhibit TCR signaling. Addition of UV-inactivated particles to primary human CD3+ T cells for 16 hrs prior to activation with anti-CD3/CD28 significantly reduced IL-2 release (FIG. 24A) and upregulation of CD69 (FIG. 24B) in a dose-related manner. YFV titer in supernatant prior to UV inactivation was 1×10⁶⁵ TCID₅₀/mL. Dose represents ml supernatant added. **=p<0.01

FIG. 25. Enhanced TCR signaling with YFV Lck predicted substrates. Jurkat cell lines were generated expressing the YFV (17D strain) envelope (YFenv) or peptide regions containing conserved tyrosines (Y274 and Y375) predicted to be Lck substrates (see top panel). Following activation with anti-CD3/CD28, the YFV (C-terminus truncated) and the Y274 inhibited IL-2 release and upregulation of CD69 compared to the Jurkat parental control, and a cell line expressing a conserved tyrosine predicted to be a substrate for Lyn (Y96). The Y375 peptide expressing cells consistently demonstrated enhanced TCR signaling.

FIG. 26. CD3+ murine splenocytes. CD3+ murine (BALB/C) splenocytes were prepared from adult mice, incubated with the Lck inhibitor II at concentrations shown prior to infection with YFV (17D). Replication was significantly higher in splenocytes incubated with Lck inhibitor.

FIG. 27. Complete HA coding sequence for A/California/25/2009(H1N1) accession GQ457514/1. Total number of predicted Lck sites in this isolate: 162, 175, 209, 214, 366, 463, 501, 528, 534.

### DESCRIPTION OF ILLUSTRATIVE EMBODIMENTS

Here, the inventors examined potential mechanisms by which GBV-C and other RNA viruses reduce immunoactivation. The have found a novel viral mechanism that inhibits T cell receptor (TCR) signaling via competition for the lymphocyte-specific protein tyrosine kinase (Lck) mediated by the persistent human Pegivirus GBV-C envelope glycoprotein E2. Additional data showing that hepatitis C virus (HCV) and yellow fever virus (YFV, 17D strain) similarly inhibit T cell activation, and the envelope glycoprotein of both of these viruses interfere with T cell activation is provided. While the Lck kinase is involved in these studies of GBV-C and HCV, additional T cell inhibitory signaling molecules are also involved for HCV. Furthermore, bioinformatic predictions for other human pathogens are provided showing that they share this immunomodulatory feature, including west Nile virus (WNV), dengue viruses (DENV), Japanese encephalitis virus (JEV), Influenza A and B, and HIV. A major problem with subunit vaccines for many of these viruses is that they are poor immunogens and elicit low levels of antibody and poor memory responses. Thus, the inventors posit that by identification of the T cell interacting domains of the envelope proteins, followed by mutation of critical amino acids required to interfere with T and B cell responses, they can generate more potent vaccines with improved longevity of protection.

GBV-C and the related HCV are the only two strictly cytoplasmic human RNA viruses that cause persistent infection. GBV-C modulates global T cell activation as determined by measurement of surface markers upregulated on CD4+ and CD8+ T cells following activation (Nattermann et al., 2003; Maidana et al., 2009; Xiang et al., 2004; Xiang et al., 2006; Schwarze-Zander et al., 2010; Stapleton, et al., 2012). The effect is modest, and GBV-C infected humans are not characterized by side effects of immunosuppression (reviewed in Bhattarai & Stapleton, 2012). In contrast, HCV is associated with an increased susceptibility to other infections, particularly HBV, bacterial infections, and schistosomiasis (reviewed in Hahn, 2003). Although anti-HCV envelope antibodies can protect chimpanzees from infection (Farci et al., 1996), immune responses to HCV envelope are weak (Fournillier et al., 2001; Cerny and Chisari, 1999). Several reasons for this have been proposed including virion or E2 association with lipids, heavy glycosylation, and marked antigenic variation (Fournillier et al., 2001).

Numerous clinical studies find an association between GBV-C infection and reduced levels of T and B cell activation (Bowen, and Walker, 2005; Lauer and Walker, 2001; Kanto et al., 1999; Krishnada et al., 2010; Kobayashi et al., 1998; Semmo et al., 2005; Eckels et al., 1999; Serti et al., 2011; Doganiuc et al., 2003; Tomova et al., 2009; Masciopinto et al., 2004). Expression of the GBV-C E2 protein in a CD4+ T cell line resulted in a block in IL-2 release, upregulation of activation markers CD69 and CD25 following stimulation through the T cell receptor (TCR) (Bhattarai et al., 2012b). Furthermore, addition of recombinant E2 to primary human CD4 and CD8 cells blocked these three measures of TCR signaling (Bhattarai et al., 2012b). Characterization of this TCR-signaling block identified three potential mechanisms by which E2 might interfere. First, there are two SH3 binding domains a small peptide region in E2, and a direct interaction between E2 and the proximal kinase Lck was demonstrated by reciprocal co-immunoprecipitation experiments. Secondly, there are two predicted Lck substrate domains in E2, and Lck phosphorylated recombinant E2 protein and these two synthetic peptides. Thirdly, deletion mapping of E2 demonstrated that the tyrosine at position 87, when expressed as a recombinant protein or synthesized as a peptide that maintained the predicted Lck binding domain, was sufficient to inhibit Lck activation and signaling. Although synthetic peptides including the downstream predicted tyrosine (Y285) are phosphorylated in vitro, neither expression of recombinant proteins including this domain nor synthetic peptides interfere with TCR signaling. Furthermore, although the SH3 binding domains may influence E2 interactions with Lck intracellularly, these are not required for inhibition. These data illustrate that prediction models alone cannot determine if a protein will serve as a functional substrate, and that further experimentation is required to prove an effect.

While GBV-C replicates in T and B lymphocytes (Xiang et al., 2000; George et al., 2006), a very low proportion of lymphocytes in peripheral blood are infected (on average, <1%). Thus, infection alone is unlikely to cause the global reduction in TCR-mediated activation. The inventors have found that serum microvesicles obtained from GBV-C-infected people block T cell activation compared to serum microvesicles from GBV-C uninfected. The inventors further show that CD4+ T cell lines expressing E2 protein produce exosomes containing E2 which reduced T cell activation. Previous studies demonstrate that HCV produces exosomes and that E2 is incorporated in these via its interactions with the E2 receptor CD81 (Masciopinto et al., 2004), a common component of exosomes.

GBV-C E2 protein is poorly immunogenic in mice (Mohr et al. 2010), and even less immunogenic in chimpanzees (unpublished data). The inventors propose that the substitution of alternative amino acids for the tyrosine at position 87 of the GBV-C E2 will enhance immunogenicity both in antibody titer and in T cell responses (including memory T cells). They also propose that the SH3 binding domains will interfere with T cell function, and that mutation of the prolines of the PXXP motif (where X is any amino acid) at GBV-C E2 positions 48, 51, 257 and 260 will further enhance immunogenicity and memory.

### I. VIRUSES

The inventors initially discovered that the GBV-C envelope glycoprotein contains binding sites and substrate sites that compete with lymphocyte kinases leading to impaired activation. Subsequently, the hepatitis C virus (HCV) and yellow fever virus (YFV) envelopes were demonstrated similarly impair lymphocyte activation. Based on bioinformatic review of sequences from RNA viruses (influenza serves as the exemplar), it is now found that this is a common feature of RNA viruses. The inventors propose that this explains the poor immunogenicity and memory responses to immunization with recombinant envelope proteins. Using these sites as immunosuppressive agents is therefore proposed Further, by identification and mutation of these immunomodulatory sites, envelope glycoproteins will be more immunogenic and will induce improved memory T and B cell responses.

As such, the invention involves two aspects, both stemming from the identification of viral envelope sequences that inhibit T cell activation. These sequences can be used reduce host immune responses in situations where such is desired, or they can be altered and then used in the context of improved vaccination to prevent or limit viral infection.

This will apply for all human and animal RNA viruses including vertebrate dsRNA viruses of the family Reoviridae, and ssRNA viruses of the families Atroviridae, Caliciviridae, HEV, Picornaviridae, Togaviridae, Flaviviridae, Coronaviridae, Orthomyxoviridae, Arenaviridae, Bunyaviridae, Paramyxoviridae, Filoviridae, Rabdoviridae, and Retroviridae.

A. Hepatitis C Virus

Hepatitis C virus (HCV) was discovered in 1989, and accounts for approximately 20% of acute hepatitis cases in the United States (Alter, 1997). About 80% of HCV infections become persistent, and 20% of these progress into chronic disease. Approximately 170 million people worldwide are infected with HCV (Conry-Cantilena et al., 1996). Due to the long period of time from infection until the development of serious liver disease, it is predicted that there will be a marked increase in liver disease resulting from HCV over the next 25 years (Williams, 1999; Seeff, 1997). In fact, surgery patients and others requiring blood transfusions, and especially those having suppressed immune systems, resulting, for example, from drugs administered in connection with organ transplantation, are at risk of developing HCV infection, which is the primary cause of transfusion-associated hepatitis in the world today. It has been estimated that posttransfusion hepatitis C may be responsible for up to 3,000 annual cases of chronic active hepatitis or cirrhosis of the liver in the U.S. alone. Hemodialysis patients, as well as intravenous drug abusers are other groups which are at risk for acquiring HCV infection.

Various clinical studies have been conducted with the goal of identifying pharmaceutical agents capable of effectively treating HCV infection in patients afflicted with chronic hepatitis C. These studies have involved the use of dideoxynucleoside analogues and interferon-α, alone and in combination therapy with other anti-viral substances (U.S. Pat. No. 5,633,388). Such studies have shown, however, that substantial numbers of the participants do not respond to this therapy, and of those that do respond favorably, a large proportion were found to relapse after termination of treatment.

HCV primarily replicates in the hepatocyte (Major et al., 1997), but is also found in association with a variety of peripheral blood cells (PBC's) (Major et al., 1997; Schmidt et al., 1997). Although controversial, it appears that HCV replicates to some extent in PBCs, and inefficient in vitro cultivation can be achieved in T- and B-cell lines (Major et al., 1997; Bartenschlager et al., 2000).

The mechanisms by which HCV attaches and enters cells has not been clear. Two cellular surface receptors have been shown to interact with HCV or the HCV envelope glycoprotein E2 in vitro, leading to speculation that either may represent the HCV cellular receptor (Pileri et al., 1998; Monazahian et al., 1999; Agnello et al., 1999; Flint et al., 1999; Wuenschmann et al., 2000). It has been shown that recombinant HCV E2 binds to human CD81 (Pileri et al., 1998; Flint et al., 1999; Flint and Maidens et al., 1999; Hadlock et al., 2000; Owsianka et al., 2001; Flint and McKeating, 2000; Petracca et al., 2000; Patel et al., 2000). CD81 is a member of the tetraspanin superfamily of cell surface molecules, and is expressed on virtually all nucleated cells (Levy and Maecker, 1998). Initial studies suggested that E2 binding to CD81 may be responsible for the binding of HCV to target cells in vivo. However, although E2 has repeatedly been shown to bind CD81, only two studies presented evidence that HCV particles derived from human serum bind to this surface molecule (Pileri et al., 1998; Hadlock et al., 2000).

The inventors have showed that, although HCV E2 binds specifically to CD81 (Wuenschmann et al., 2000), the binding of HCV particles purified from plasma was not inhibited by soluble CD81, and the extent of virus binding correlated with the level of LDLr expression (Wuenschmann et al., 2000). Additional lines of evidence argue that CD81 is not the HCV receptor. HCV E2 has a higher affinity for marmoset CD81 than human CD81, yet marmosets are not susceptible to HCV. The affinity for HCV E2 to CD81 was found to be significantly lower than predicted for a true viral receptor (Petracca et al., 2000). Using an RT-PCR based detection method, plasma-derived HCV and HCV E2 bound to U937 subcloned cells that lack expression of CD81 (Hamaia and Allain, 2001). These data suggest that CD81 is not the primary cell receptor for HCV.

Nevertheless, HCV E2 does interact with CD81, and the E2 regions involved in CD81 binding are highly conserved (Pileri et al., 1998; Flint et al., 1999; Flint and Maidens et al., 1999; Hadlock et al., 2000; Owsianka et al., 2001; Flint and McKeating, 2000; Petracca et al., 2000; Patel et al., 2000)), suggesting a functional role for CD81-E2 interactions in HCV replication (Pileri et al., 1998; Flint et al., 1999; Flint and Maidens et al., 1999; Hadlock et al., 2000; Owsianka et al., 2001; Flint and McKeating, 2000). The extremely low density of HCV found in gradient centrifugation of infectious serum suggested an association with VLDL and LDL (Hijikata et al., 1993; Bradley et al., 1991; Prince et al., 1996). Infectious virus was found at the same densities as VLDL and LDL and coprecipitated with LDL (Monazahian et al., 1999; Bradley et al., 1991; Prince et al., 1996; Thomssen and Thiele, 1993; Xiang et al., 1998). Subsequent studies (Monazahian et al., 1999; Bradley et al., 1991; Prince et al. 1996; Xiang et al., 1998) demonstrated an interaction between HCV or HCV-LDL complexes with the low density lipoprotein receptor (LDLr) (Wuenschmann et al., 2000; Prince et al., 1996; Thomssen and Thiele, 1993; Xiang et al., 1998; Thomssen et al., 1992).

HCV present in the plasma of infected people has also been shown to interact with very-low-density (VLDL) and low-density lipoproteins (LDL). The liver synthesizes VLDL which consists of triaglycerols, cholesterol, phospholipids and the apoprotein apoB-100, VLDL's released into the blood, where it acquires additional lipoproteins C.sub.II and apoE from high-density lipoproteins (HDL). VLDL is digested by Lipoprotein Lipase (LPL), an enzyme found attached to capillary endothelial cells, to form intermediate density lipoproteins (IDL) and LDL, and apoB-100 is the only remaining apoprotein in LDL. The low-density lipoprotein receptor (LDLr) recognizes both apoE and apoB-100 and can therefore bind VLDL, IDL and chylomicron remnants in addition to LDL. (Marks et al., 1996).

HCV-RNA containing material in serum, presumably virus particles, separate into very low density particles (<1.06 g/cm³) by gradient sedimentation, suggesting that HCV associates with VLDL and LDL (Monazahian et al., 1999; Thomssen et al., 1993; Xiang et al., 1998; Prince et al., 1996; Bradley et al., 1991). In addition, particles with densities of 1.11-1.18 g/cm³ have been described (Xiang et al., 1998; Prince et al., 1996; Bradley et al., 1991; Hijikata et al., 1993). Chimpanzee infectivity studies demonstrated that the very low density HCV particles were highly infectious, whereas the particles of higher density were not infectious (Bradley, 2000). (Monazahian et al., 1999; Xiang et al., 1998; Prince et al., 1996; Bradley et al., 1991). Thomssen et al. (1993) showed that HCV coprecipitated with LDL and demonstrated an interaction of HCV or HCV-LDL complexes with the LDLr (Wuenschmann et al., 2000; Thomssen et al., 1993; Xiang et al., 1998; Prince et al., 1996; Thomssen et al., 1992).

Monazahian et al. (1999) demonstrated that expression of recombinant human LDLr in murine cells lacking human CD81 confirmed binding of HCV to these cells (Monazahian et al., 1999) and Agnello et al. (1999) demonstrated that HCV bound to and entered fibroblasts containing LDLr, but not LDLr deficient fibroblasts, using an in situ hybridization method (Agnello et al., 1999). Using flow cytometry, the inventors confirmed that plasma-derived HCV bound to cells expressing LDLr, but not to cells lacking the LDLr (Wuenschmann et al., 2000). No interactions between viral envelope proteins (E1 or E2) and the LDL receptor have been reported (Wuenschmann et al., 2000). However, Monazahian et al. (1999) found that in vitro translated HCV E1 and E2 proteins, labeled with ³⁵S-methionine co-precipitated with VLDL, LDL and HDL (Monazahian et al., 2000).

HCV E2 is the outer protein of the viral envelope and may participate in the binding of viruses to the target cells. The protein starts at amino acid 394 of the HCV polyprotein, and extends to amino acid 747. It has a hypervariable region at the amino terminus of the protein, and the carboxy terminus includes a transmembrane domain.

Due to the deficiencies in the prior art, there remains a need for more effective treatments to lower LDL levels in a subject. There also remains a need for new and useful methods of reducing or preventing HCV infection in a subject. The presently claimed invention overcomes the deficiencies in the prior art by disclosing new and useful methods for reducing LDL levels in a subject. The present invention also discloses new and useful methods of identifying HCV inhibitors and methods of treating HCV infection.

The viral genomic sequence of HCV is known, as are methods for obtaining the sequence. See, International Publication Nos. WO 89/04669; WO 90/11089; and WO 90/14436. Hepatitis C Virus (HCV) HCV is an enveloped virus containing a positive-sense single-stranded RNA genome of approximately 9.5 kb. The genomic sequence of HCV is approximately 9401 base pairs in length (SEQ ID NO: 1). The peptide sequence for HCV can be obtained from Genbank Accession No. M62321. The viral genome consists of a lengthy 5′ untranslated region (UTR), a long open reading frame encoding a polyprotein precursor of approximately 3011 amino acids (SEQ ID NO: 2) and a short 3′ UTR. The 5′ UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (IRES). The polyprotein precursor is cleaved by both host and viral proteases to yield mature viral structural and non-structural proteins. Viral structural proteins include a nucleocapsid core protein and two envelope glycoproteins, E1 and E2 (U.S. Pat. No. 6,326,151).

HCV utilizes the low density lipoprotein receptor (LDLr) for cell binding and entry (Wuenschmann et al., 2000; Monazahian et al., 1999; Agello et al., 1999). The inventors have previously reported that the HCV envelope glycoprotein (HCV E2 glycoprotein) binds to the lipid moiety of human lipoproteins, and the lipid-virus complex uses the natural receptor for LDL to bind to cells. The HCV E2 glycoprotein starts at amino acid 394 of the HCV polyprotein, and extends to amino acid 747. It has a hypervariable region at the amino terminus of the protein, and the carboxy terminus includes a transmembrane domain. HCV enters the cell via endocytosis using the LDL receptor. HCV E2 glycoprotein interactions with LDL result not only in CD81-independent binding to cells (Wuenschmann et al., 2000), but also to enhancement in LDL binding and uptake by the cells.

B. Other Viruses

1. Yellow Fever Virus

Yellow fever is an acute viral hemorrhagic disease. The virus is a 40 to 50 nm enveloped RNA virus with positive sense of the Flaviviridae family. The yellow fever virus is transmitted by the bite of female mosquitoes (the yellow fever mosquito, Aedes aegypti, and other species) and is found in tropical and subtropical areas in South America and Africa, but not in Asia. The only known hosts of the virus are primates and several species of mosquito. The origin of the disease is most likely to be Africa, from where it was introduced to South America through the slave trade in the 16th century. Since the 17th century, several major epidemics of the disease have been recorded in the Americas, Africa, and Europe. In the 19th century, yellow fever was deemed one of the most dangerous infectious diseases.

Yellow fever presents in most cases in humans with fever, chills, anorexia, nausea, muscle pain (with prominent backache) and headache, which generally subsides after several days. In some patients, a toxic phase follows, in which liver damage with jaundice (inspiring the name of the disease) can occur and lead to death. Because of the increased bleeding tendency (bleeding diathesis), yellow fever belongs to the group of hemorrhagic fevers. The WHO estimates that yellow fever causes 200,000 illnesses and 30,000 deaths every year in unvaccinated populations; today nearly 90% of the infections occur in Africa.

A safe and effective vaccine against yellow fever has existed since the middle of the 20th century, and some countries require vaccinations for travelers. Since no therapy is known, vaccination programs are of great importance in affected areas, along with measures to prevent bites and reduce the population of the transmitting mosquito. Since the 1980s, the number of cases of yellow fever has been increasing, making it a re-emerging disease. This is likely due to warfare and social disruption in several African nations.

Yellow fever begins after an incubation period of three to six days. Most cases only cause a mild infection with fever, headache, chills, back pain, loss of appetite, nausea, and vomiting. In these cases the infection lasts only three to four days. In fifteen percent of cases, however, sufferers enter a second, toxic phase of the disease with recurring fever, this time accompanied by jaundice due to liver damage, as well as abdominal pain. Bleeding in the mouth, the eyes, and the gastrointestinal tract will cause vomitus containing blood (hence the Spanish name for yellow fever, vomito negro (black vomit)). The toxic phase is fatal in approximately 20% of cases, making the overall fatality rate for the disease 3% (15%*20%). In severe epidemics, the mortality may exceed 50%.

Yellow fever is caused by the yellow fever virus, a 40 to 50 nm wide enveloped RNA virus belonging to the family Flaviviridae. The positive sense single-stranded RNA is approximately 11,000 nucleotides long and has a single open reading frame encoding a polyprotein. Host proteases cut this polyprotein into three structural (C, prM, E) and seven non-structural proteins (NS1, NS2A, NS2B, NS3, NS4A, NS4B, NS5); the enumeration corresponds to the decreased pH induces the fusion of the endosomal membrane with the virus envelope. Thus, the capsid reaches the cytosol, decays and releases the genome. Receptor binding as well as membrane fusion are catalyzed by the protein E, which changes its conformation at low pH, which causes a rearrangement of the 90 homodimers to 60 homotrimers.

After entering the host cells, the viral genome is replicated in the rough endoplasmic reticulum (ER) and in the so-called vesicle packets. At first, an immature form of the virus particle is produced inside the ER, whose M-protein is not yet cleaved to its mature form and is therefore denoted as prM (precursor M) and forms a complex with protein E. The immature particles are processed in the Golgi apparatus by the host protein furin, which cleaves prM to M. This releases E from the complex which can now take its place in the mature, infectious virion.

The yellow fever virus is mainly transmitted through the bite of the yellow fever mosquito Aedes aegypti, but other mosquitoes such as the “tiger mosquito” (Aedes albopictus) can also serve as a vector for the virus. Like other Arboviruses which are transmitted via mosquitoes, the yellow fever virus is taken up by a female mosquito which sucks the blood of an infected person or primate. Viruses reach the stomach of the mosquito, and if the virus concentration is high enough, the virions can infect epithelial cells and replicate there. From there they reach the haemocoel (the blood system of mosquitoes) and from there the salivary glands. When the mosquito next sucks blood, it injects its saliva into the wound, and thus the virus reaches the blood of the bitten person. There are also indications for transovarial and transstadial transmission of the yellow fever virus within A. aegypti, i.e., the transmission from a female mosquito to her eggs and then larvae. This infection of vectors without a previous blood meal seems to play a role in single, sudden breakouts of the disease.

For journeys into affected areas, vaccination is highly recommended, since mostly non-native people suffer severe cases of yellow fever. The protective effect is established 10 days after vaccination in 95 percent of the vaccinated people and lasts for at least 10 years (even 30 years later, 81% of patients retained immunity). The attenuated live vaccine (stem 17D) was developed in 1937 by Max Theiler from a diseased patient in Ghana and is produced in chicken eggs. The WHO recommends routine vaccinations for people living in endemic areas between the 9th and 12th month after birth. In about 20% of all cases, mild, flu-like symptoms may develop.

In rare cases (less than one in 200,000 to 300,000), the vaccination can cause YEL-AVD (yellow fever vaccine-associated viscerotropic disease), which is fatal in 60% of all cases. It is probably due to a genetic defect in the immune system. But in some vaccination campaigns, a 20-fold higher incidence rate has been reported. Age is an important risk factor; in children, the complication rate is less than one case per 10 million vaccinations. Another possible side effect is an infection of the nervous system that occurs in one in 200,000 to 300,000 of all cases, causing YEL-AND (yellow fever vaccine-associated neurotropic disease), which can cause meningoencephalitis and is fatal in less than 5% of all cases.

In 2009, the largest mass vaccination against yellow fever began in West Africa, specifically Benin, Liberia, and Sierra Leone. When it is completed in 2015, more than 12 million people will have been vaccinated against the disease. According to the World Health Organization (WHO), the mass vaccination cannot eliminate yellow fever because of the vast number of infected mosquitoes in urban areas of the target countries, but it will significantly reduce the number of people infected. The WHO plans to continue the vaccination campaign in another five African countries—Central African Republic, Ghana, Guinea, Côte d'Ivoire, and Nigeria—and stated that approximately 160 million people in the continent could be at risk unless the organization acquires additional funding to support widespread vaccinations.

2. HIV

Human immunodeficiency virus (HIV) is a lentivirus (slowly-replicating retrovirus) that causes acquired immunodeficiency syndrome (AIDS), a condition in humans in which progressive failure of the immune system allows life-threatening opportunistic infections and cancers to thrive. Infection with HIV occurs by the transfer of blood, semen, vaginal fluid, pre-ejaculate, or breast milk. Within these bodily fluids, HIV is present as both free virus particles and virus within infected immune cells.

HIV infects vital cells in the human immune system such as helper T cells (specifically CD4⁺ T cells), macrophages, and dendritic cells. HIV infection leads to low levels of CD4⁺ T cells through a number of mechanisms including: apoptosis of uninfected bystander cells, direct viral killing of infected cells, and killing of infected CD4⁺ T cells by CD8 cytotoxic lymphocytes that recognize infected cells. When CD4⁺ T cell numbers decline below a critical level, cell-mediated immunity is lost, and the body becomes progressively more susceptible to opportunistic infections.

HIV is a member of the genus Lentivirus, part of the family of Retroviridae. Lentiviruses have many morphologies and biological properties in common. Many species are infected by lentiviruses, which are characteristically responsible for long-duration illnesses with a long incubation period. Lentiviruses are transmitted as single-stranded, positive-sense, enveloped RNA viruses. Upon entry into the target cell, the viral RNA genome is converted (reverse transcribed) into double-stranded DNA by a virally encoded reverse transcriptase that is transported along with the viral genome in the virus particle. The resulting viral DNA is then imported into the cell nucleus and integrated into the cellular DNA by a virally encoded integrase and host co-factors. Once integrated, the virus may become latent, allowing the virus and its host cell to avoid detection by the immune system. Alternatively, the virus may be transcribed, producing new RNA genomes and viral proteins that are packaged and released from the cell as new virus particles that begin the replication cycle anew.

Two types of HIV have been characterized: HIV-1 and HIV-2. HIV-1 is the virus that was initially discovered and termed both LAV and HTLV-III. It is more virulent, more infective, and is the cause of the majority of HIV infections globally. The lower infectivity of HIV-2 compared to HIV-1 implies that fewer of those exposed to HIV-2 will be infected per exposure. Because of its relatively poor capacity for transmission, HIV-2 is largely confined to West Africa.

HIV is different in structure from other retroviruses. It is roughly spherical with a diameter of about 120 nm, around 60 times smaller than a red blood cell, yet large for a virus. It is composed of two copies of positive single-stranded RNA that codes for the virus's nine genes enclosed by a conical capsid composed of 2,000 copies of the viral protein p24. The single-stranded RNA is tightly bound to nucleocapsid proteins, p7, and enzymes needed for the development of the virion such as reverse transcriptase, proteases, ribonuclease and integrase. A matrix composed of the viral protein p17 surrounds the capsid ensuring the integrity of the virion particle.

This is, in turn, surrounded by the viral envelope that is composed of two layers of fatty molecules called phospholipids taken from the membrane of a human cell when a newly formed virus particle buds from the cell. Embedded in the viral envelope are proteins from the host cell and about 70 copies of a complex HIV protein that protrudes through the surface of the virus particle. This protein, known as Env, consists of a cap made of three molecules called glycoprotein (gp) 120, and a stem consisting of three gp41 molecules that anchor the structure into the viral envelope. This glycoprotein complex enables the virus to attach to and fuse with target cells to initiate the infectious cycle. Both these surface proteins, especially gp120, have been considered as targets of future treatments or vaccines against HIV.

The RNA genome consists of at least seven structural landmarks (LTR, TAR, RRE, PE, SLIP, CRS, and INS), and nine genes (gag, pol, and env, tat, rev, nef, vif, vpr, vpu, and sometimes a tenth tev, which is a fusion of tat env and rev), encoding 19 proteins. Three of these genes, gag, pol, and env, contain information needed to make the structural proteins for new virus particles. For example, env codes for a protein called gp160 that is broken down by a cellular protease to form gp120 and gp41. The six remaining genes, tat, rev, nef, vif, vpr, and vpu (or vpx in the case of HIV-2), are regulatory genes for proteins that control the ability of HIV to infect cells, produce new copies of virus (replicate), or cause disease.

The two Tat proteins (p16 and p14) are transcriptional trans activators for the LTR promoter acting by binding the TAR RNA element. The TAR may also be processed into microRNAs that regulate the apoptosis genes ERCC1 and IER3. The Rev protein (p19) is involved in shuttling RNAs from the nucleus and the cytoplasm by binding to the RRE RNA element. The Vif protein (p23) prevents the action of APOBEC3G (a cell protein that deaminates DNA:RNA hybrids and/or interferes with the Pol protein). The Vpr protein (p14) arrests cell division at G2/M. The Nef protein (p27) down-regulates CD4 (the major viral receptor), as well as the MHC class I and class II molecules.

Nef also interacts with SH3 domains. The Vpu protein (p16) influences the release of new virus particles from infected cells. The ends of each strand of HIV RNA contain an RNA sequence called the long terminal repeat (LTR). Regions in the LTR act as switches to control production of new viruses and can be triggered by proteins from either HIV or the host cell. The Psi element is involved in viral genome packaging and recognized by Gag and Rev proteins. The SLIP element (TTTTTT) is involved in the frameshift in the Gag-Pol reading frame required to make functional Pol.

HIV differs from many viruses in that it has very high genetic variability. This diversity is a result of its fast replication cycle, with the generation of about 10¹⁰ virions every day, coupled with a high mutation rate of approximately 3×10⁻⁵ per nucleotide base per cycle of replication and recombinogenic properties of reverse transcriptase. This complex scenario leads to the generation of many variants of HIV in a single infected patient in the course of one day. This variability is compounded when a single cell is simultaneously infected by two or more different strains of HIV. When simultaneous infection occurs, the genome of progeny virions may be composed of RNA strands from two different strains. This hybrid virion then infects a new cell where it undergoes replication. As this happens, the reverse transcriptase, by jumping back and forth between the two different RNA templates, will generate a newly synthesized retroviral DNA sequence that is a recombinant between the two parental genomes. This recombination is most obvious when it occurs between subtypes.

The closely related simian immunodeficiency virus (SIV) has evolved into many strains, classified by the natural host species. SIV strains of the African green monkey (SIVagm) and sooty mangabey (SIVsmm) are thought to have a long evolutionary history with their hosts. These hosts have adapted to the presence of the virus, which is present at high levels in the host's blood but evokes only a mild immune response, does not cause the development of simian AIDS, and does not undergo the extensive mutation and recombination typical of HIV infection in humans.

In contrast, when these strains infect species that have not adapted to SIV (“heterologous” hosts such as rhesus or cynomologus macaques), the animals develop AIDS and the virus generates genetic diversity similar to what is seen in human HIV infection. Chimpanzee SIV (SIVcpz), the closest genetic relative of HIV-1, is associated with increased mortality and AIDS-like symptoms in its natural host. SIVcpz appears to have been transmitted relatively recently to chimpanzee and human populations, so their hosts have not yet adapted to the virus. This virus has also lost a function of the Nef gene that is present in most SIVs; without this function, T cell depletion is more likely, leading to immunodeficiency.

Three groups of HIV-1 have been identified on the basis of differences in the envelope (env) region: M, N, and O. Group M is the most prevalent and is subdivided into eight subtypes (or clades), based on the whole genome, which are geographically distinct. The most prevalent are subtypes B (found mainly in North America and Europe), A and D (found mainly in Africa), and C (found mainly in Africa and Asia); these subtypes form branches in the phylogenetic tree representing the lineage of the M group of HIV-1. Coinfection with distinct subtypes gives rise to circulating recombinant forms (CRFs). In 2000, the last year in which an analysis of global subtype prevalence was made, 47.2% of infections worldwide were of subtype C, 26.7% were of subtype A/CRF02_AG, 12.3% were of subtype B, 5.3% were of subtype D, 3.2% were of CRF_AE, and the remaining 5.3% were composed of other subtypes and CRFs. Most HIV-1 research is focused on subtype B; few laboratories focus on the other subtypes. The existence of a fourth group, “P”, has been hypothesised based on a virus isolated in 2009. The strain is apparently derived from gorilla SIV (SIVgor), first isolated from western lowland gorillas in 2006. The genetic sequence of HIV-2 is only partially homologous to HIV-1 and more closely resembles that of SIVsmm.

3. Influenza

The etiological cause of influenza, the Orthomyxoviridae family of viruses, was first discovered in pigs by Richard Shope in 1931. This discovery was shortly followed by the isolation of the virus from humans by a group headed by Patrick Laidlaw at the Medical Research Council of the United Kingdom in 1933. However, it was not until Wendell Stanley first crystallized tobacco mosaic virus in 1935 that the non-cellular nature of viruses was appreciated.

The first significant step towards preventing influenza was the development in 1944 of a killed-virus vaccine for influenza by Thomas Francis, Jr. This built on work by Australian Frank Macfarlane Burnet, who showed that the virus lost virulence when it was cultured in fertilized hen's eggs. Application of this observation by Francis allowed his group of researchers at the University of Michigan to develop the first influenza vaccine, with support from the U.S. Army. The Army was deeply involved in this research due to its experience of influenza in World War I, when thousands of troops were killed by the virus in a matter of months.

Although there were scares in the State of New Jersey in 1976 (with the Swine Flu), worldwide in 1977 (with the Russian Flu), and in Hong Kong and other Asian countries in 1997 (with H5N1 avian influenza), there have been no major pandemics since the 1968 Hong Kong Flu Immunity to previous pandemic influenza strains and vaccination may have limited the spread of the virus and may have helped prevent further pandemics. The influenza virus is an RNA virus of the family Orthomyxoviridae, which comprises five genera: Influenzavirus A, Influenzavirus B, Influenzavirus C, Isavirus and Thogotovirus. The Influenzavirus A genus has one species, influenza A virus. Wild aquatic birds are the natural hosts for a large variety of influenza A. Occasionally, viruses are transmitted to other species and may then cause devastating outbreaks in domestic poultry or give rise to human influenza pandemics. The type A viruses are the most virulent human pathogens among the three influenza types and cause the most severe disease. The influenza A virus can be subdivided into different subtypes based on the antibody response to these viruses.

Influenzaviruses A, B and C are very similar in structure. The virus particle is 80-120 nanometres in diameter and usually roughly spherical, although filamentous forms can occur. This particle is made of a viral envelope containing two main types of glycoproteins, wrapped around a central core. The central core contains the viral RNA genome and other viral proteins that package and protect this RNA. Unusually for a virus, its genome is not a single piece of nucleic acid; instead, it contains seven or eight pieces of segmented negative-sense RNA. The Influenza A genome encodes 11 proteins: hemagglutinin (HA), neuraminidase (NA), nucleoprotein (NP), M1, M2, NS1, NS2(NEP), PA, PB1, PB1-F2 and PB2.

Hemagglutinin (HA) and neuraminidase (NA) are the two large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins are targets for antiviral drugs. Furthermore, they are antigens to which antibodies can be raised. Influenza A viruses are classified into subtypes based on antibody responses to HA and NA. These different types of HA and NA form the basis of the H and N distinctions in, for example, H5N1.

Influenza viruses bind through hemagglutinin onto sialic acid sugars on the surfaces of epithelial cells; typically in the nose, throat and lungs of mammals and intestines of birds. The cell imports the virus by endocytosis. In the acidic endosome, part of the hemagglutinin protein fuses the viral envelope with the vacuole's membrane, releasing the viral RNA (vRNA) molecules, accessory proteins and RNA-dependent RNA polymerase into the cytoplasm. These proteins and vRNA form a complex that is transported into the cell nucleus, where the RNA-dependent RNA polymerase begins transcribing complementary positive-sense vRNA. The vRNA is either exported into the cytoplasm and translated, or remains in the nucleus. Newly-synthesised viral proteins are either secreted through the Golgi apparatus onto the cell surface or transported back into the nucleus to bind vRNA and form new viral genome particles. Other viral proteins have multiple actions in the host cell, including degrading cellular mRNA and using the released nucleotides for vRNA synthesis and also inhibiting translation of host-cell mRNAs.

Negative-sense vRNAs that form the genomes of future viruses, RNA-dependent RNA polymerase, and other viral proteins are assembled into a virion. Hemagglutinin and neuraminidase molecules cluster into a bulge in the cell membrane. The vRNA and viral core proteins leave the nucleus and enter this membrane protrusion. The mature virus buds off from the cell in a sphere of host phospholipid membrane, acquiring hemagglutinin and neuraminidase with this membrane coat. As before, the viruses adhere to the cell through hemagglutinin; the mature viruses detach once their neuraminidase has cleaved sialic acid residues from the host cell. After the release of new influenza viruses, the host cell dies.

Because of the absence of RNA proofreading enzymes, the RNA-dependent RNA polymerase makes a single nucleotide insertion error roughly every 10 thousand nucleotides, which is the approximate length of the influenza vRNA. Hence, the majority of newly-manufactured influenza viruses are mutants, causing “antigenic drift.” The separation of the genome into eight separate segments of vRNA allows mixing or reassortment of vRNAs if more than one viral line has infected a single cell. The resulting rapid change in viral genetics produces antigenic shifts and allows the virus to infect new host species and quickly overcome protective immunity.

4. Other Viruses

The present invention contemplates the use of peptides and polypeptides deriving from other envelope proteins including West Nile virus, Japanese Encephalitis virus, Dengue virus and Classical Swine Fever virus (CSFV).

### II. VIRAL POLYPEPTIDES AS IMMUNOSUPPRESSIVE AGENTS

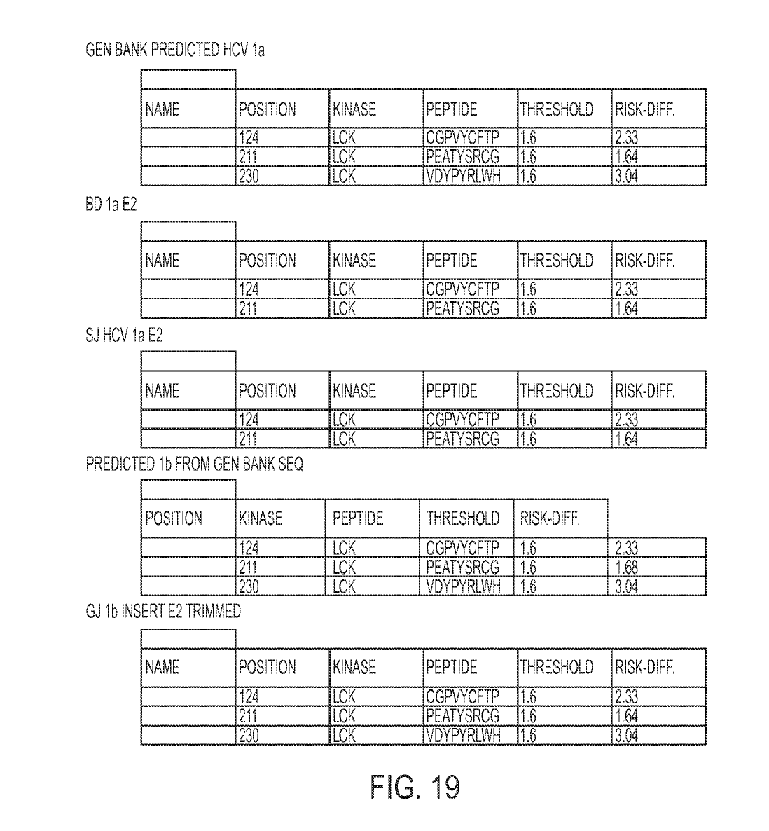

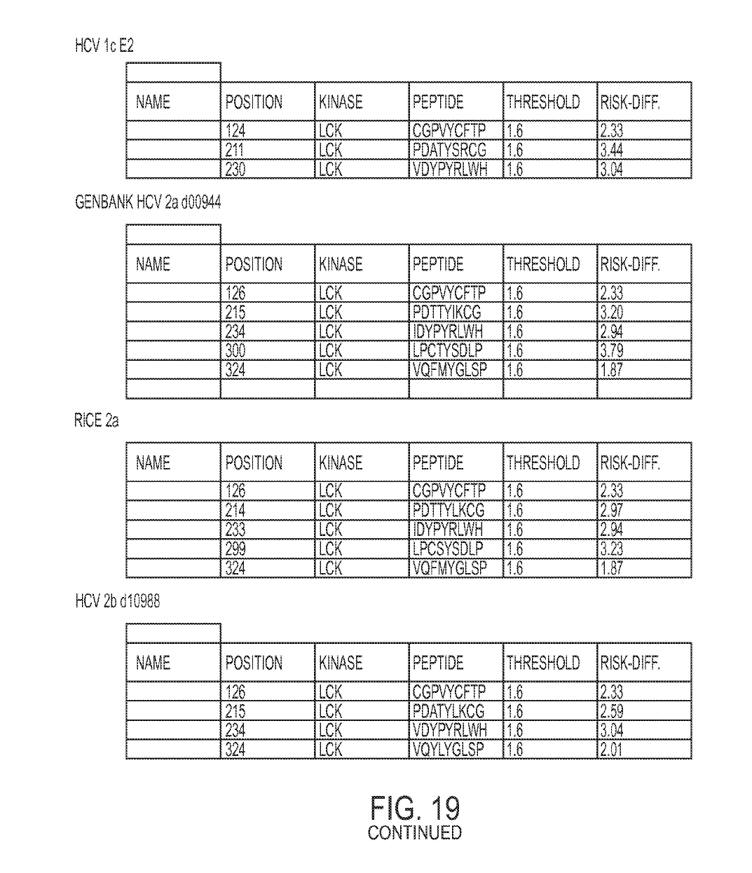

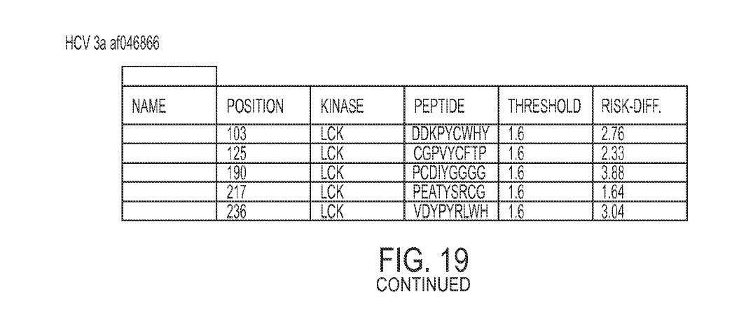

In certain aspects, the invention is directed to viral envelope proteins, e.g., HCV E2 protein. The expression or provision of peptides and polypeptides can be used to modulate immune function. It is contemplated that the compositions and methods disclosed herein may be utilized to express all or part of the protein and derivates thereof. In certain embodiments, compositions of the invention may include the nucleic acids encoding the peptides as set forth in FIGS. 19-21. The method of claim 1, wherein said peptide comprises about 10, 11, 12, 13, 14, 15, 16, 17, 18, 19, 20, 21, 22, 23, 24, 25, 30, 35, 40, 45, 50, 75, 100, 150, 175, 200, 219, 250 consecutive residues of an envelope sequence. Determination of which peptides possess activity may be achieved using functional assays measuring T-cell activation and proliferation as well as cytokine productiOn, which are familiar to those of skill in the art.

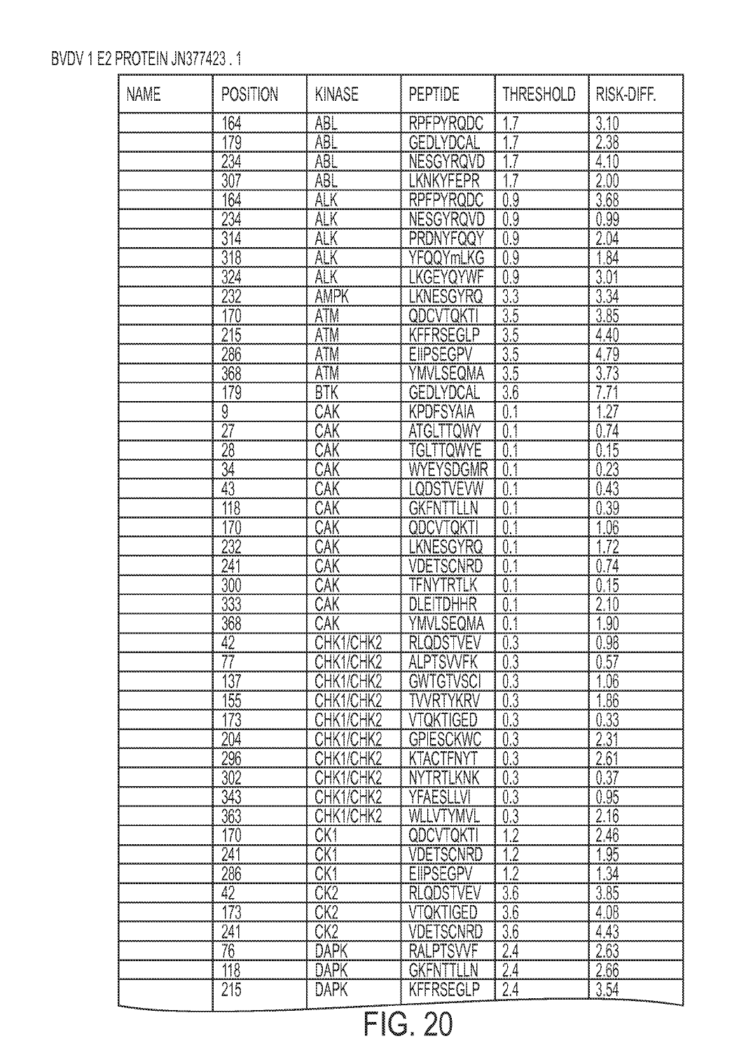

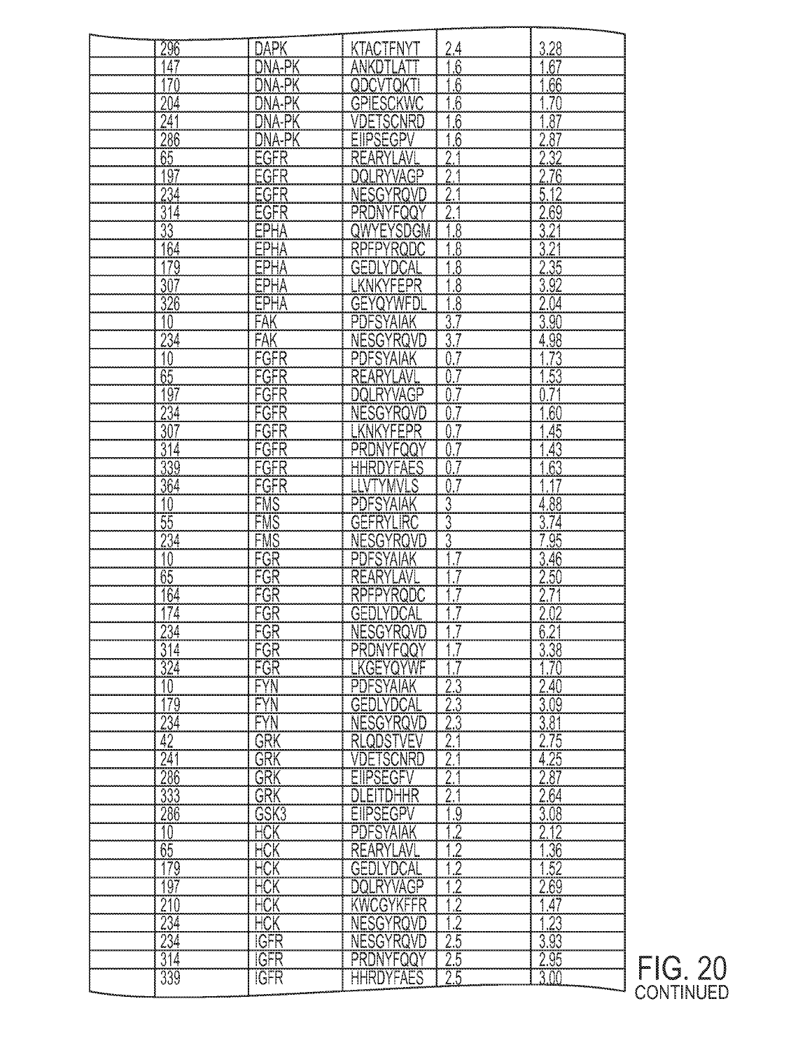

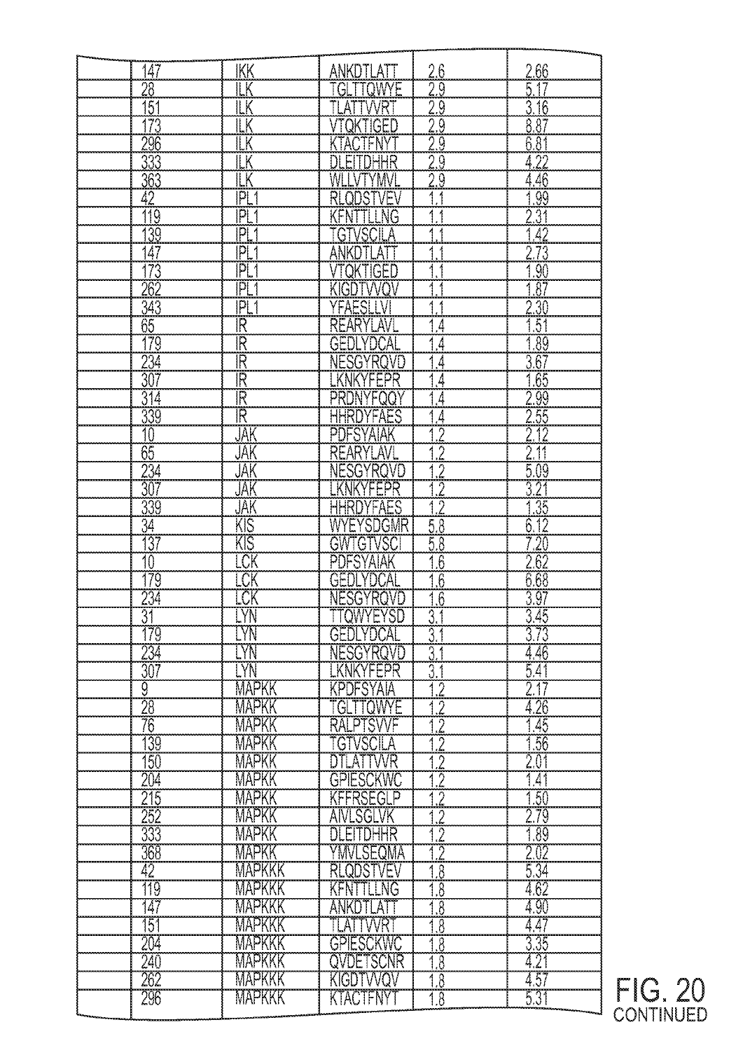

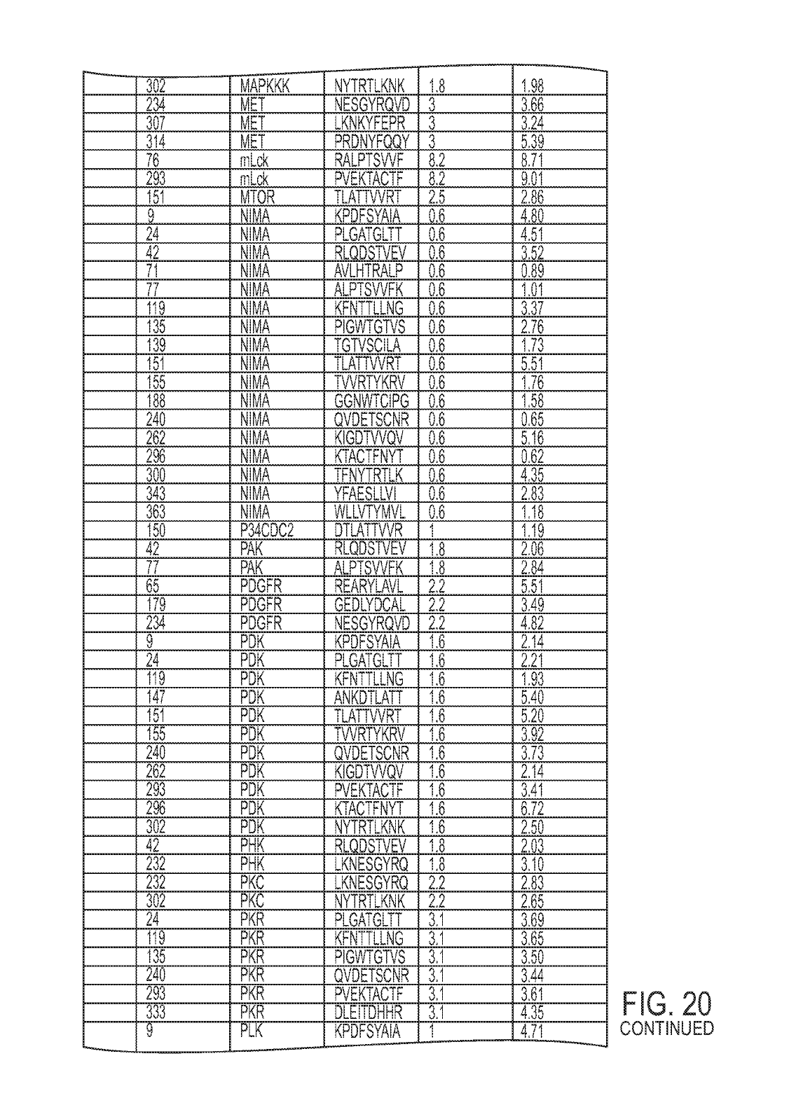

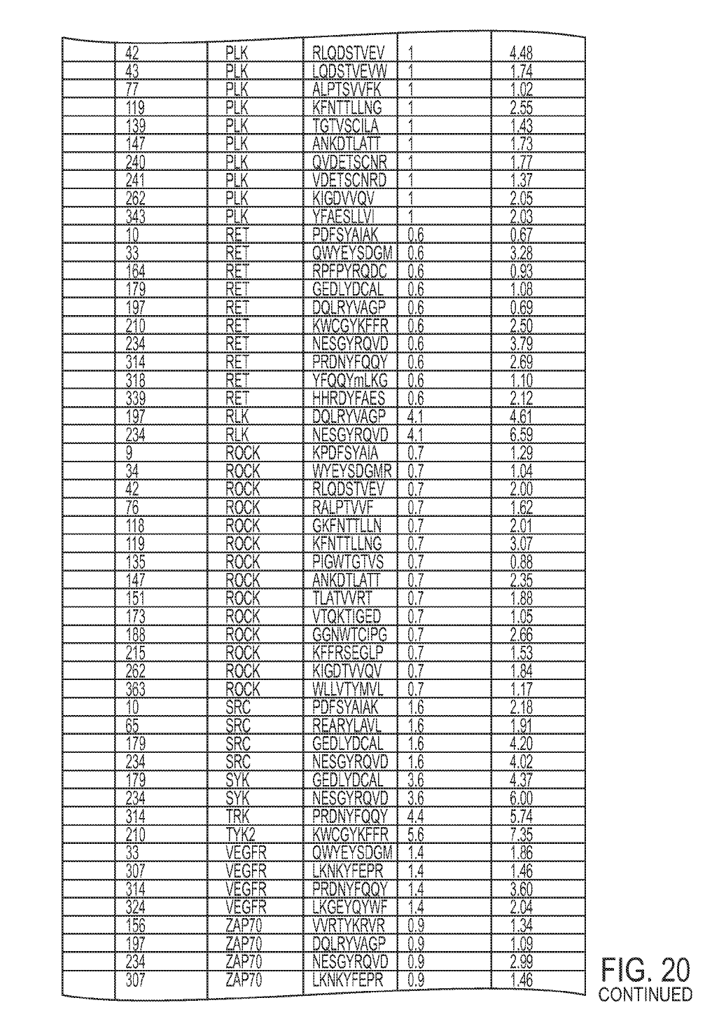

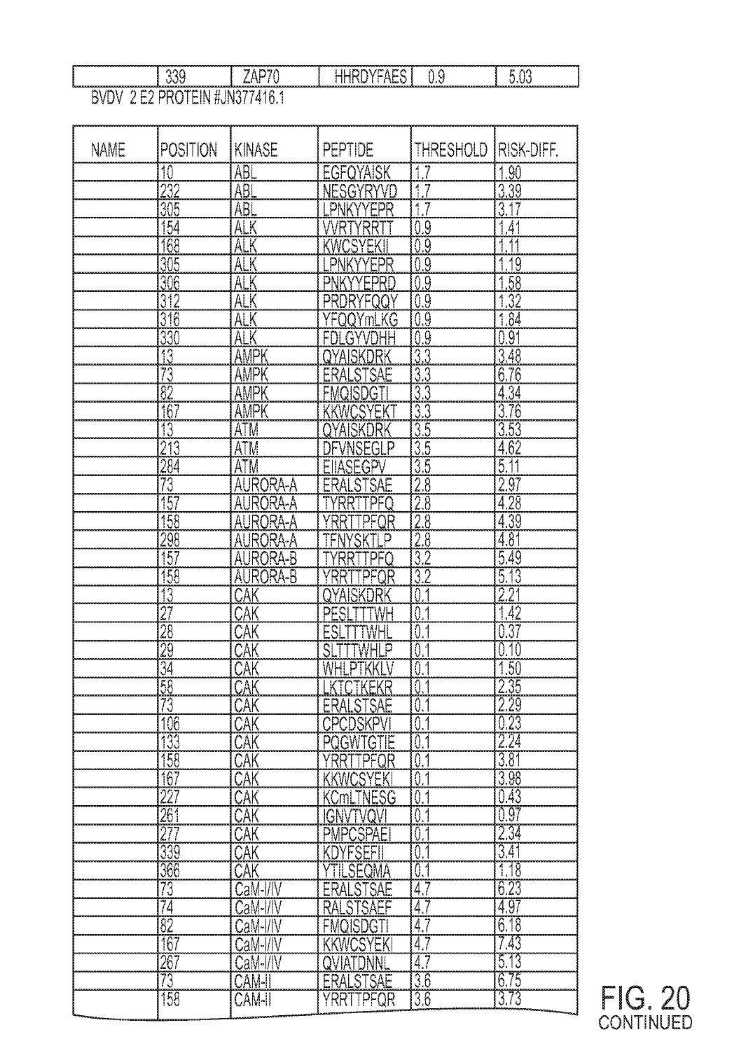

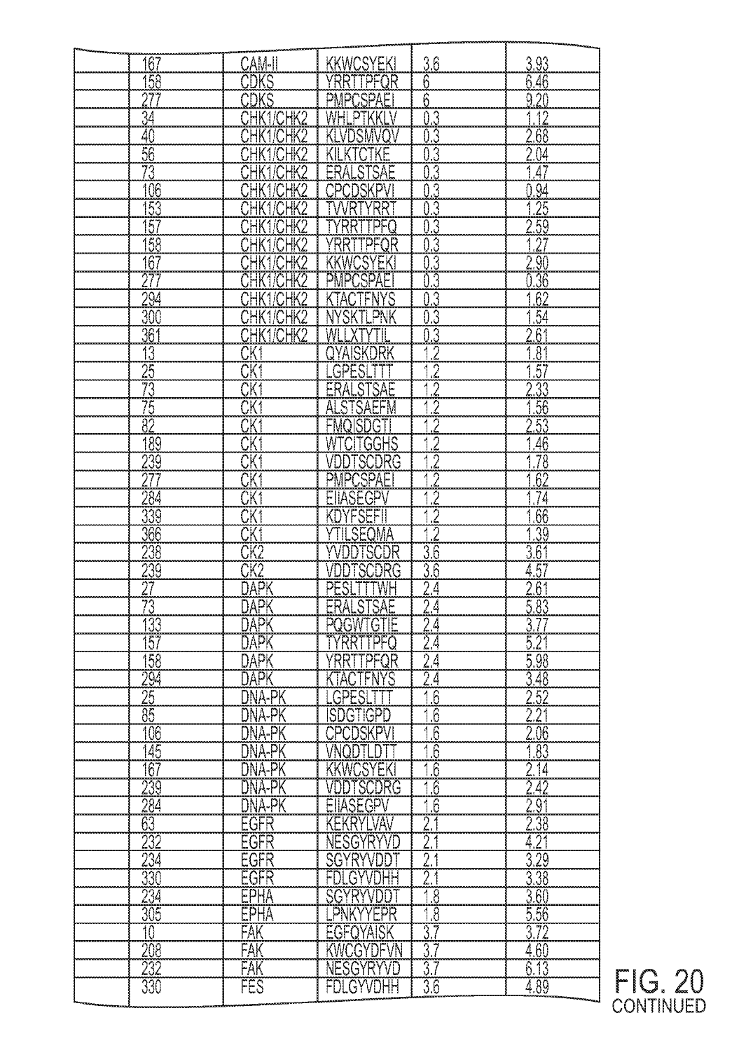

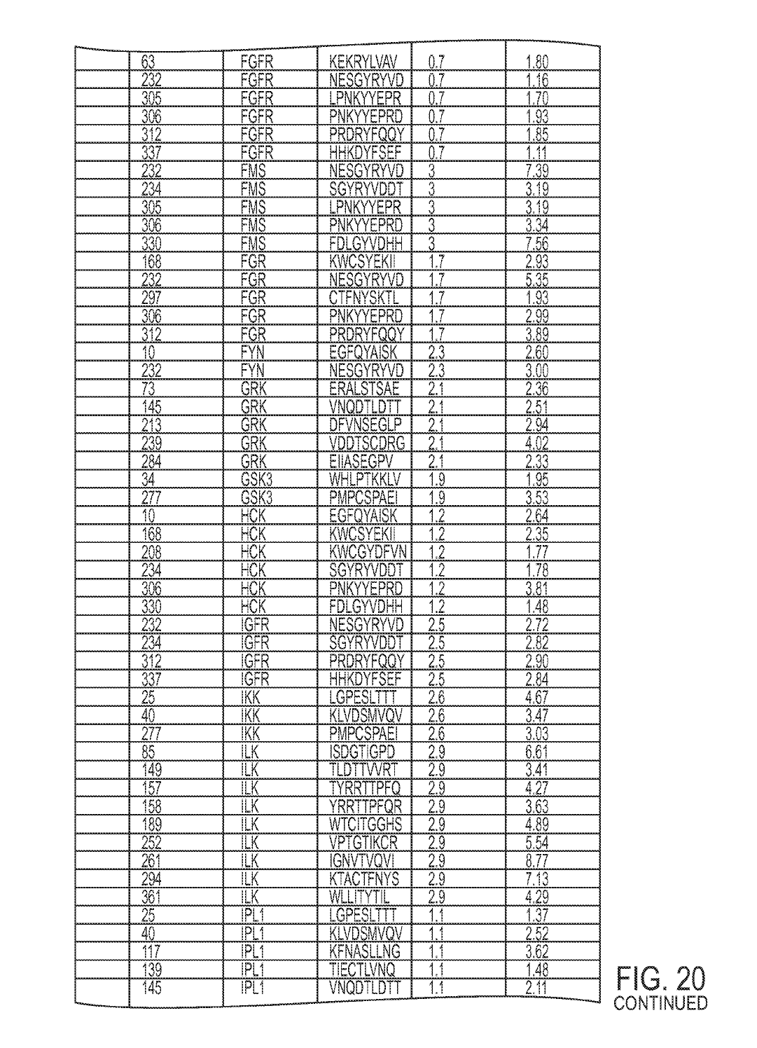

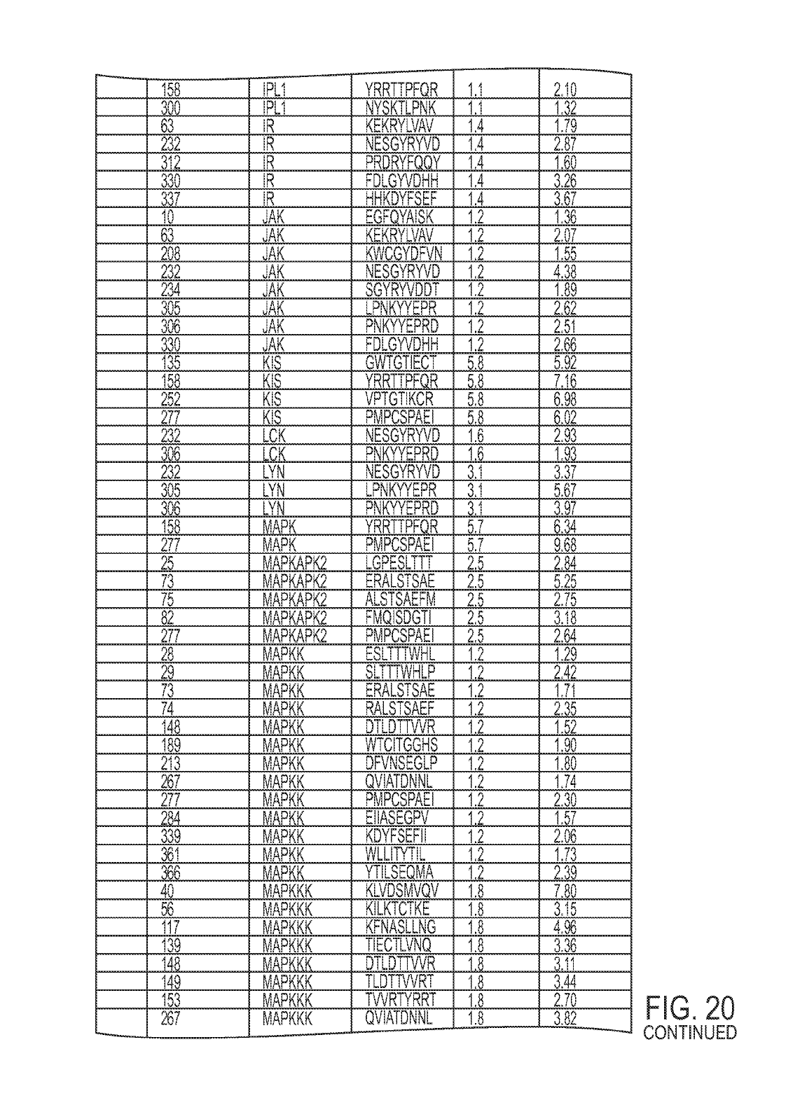

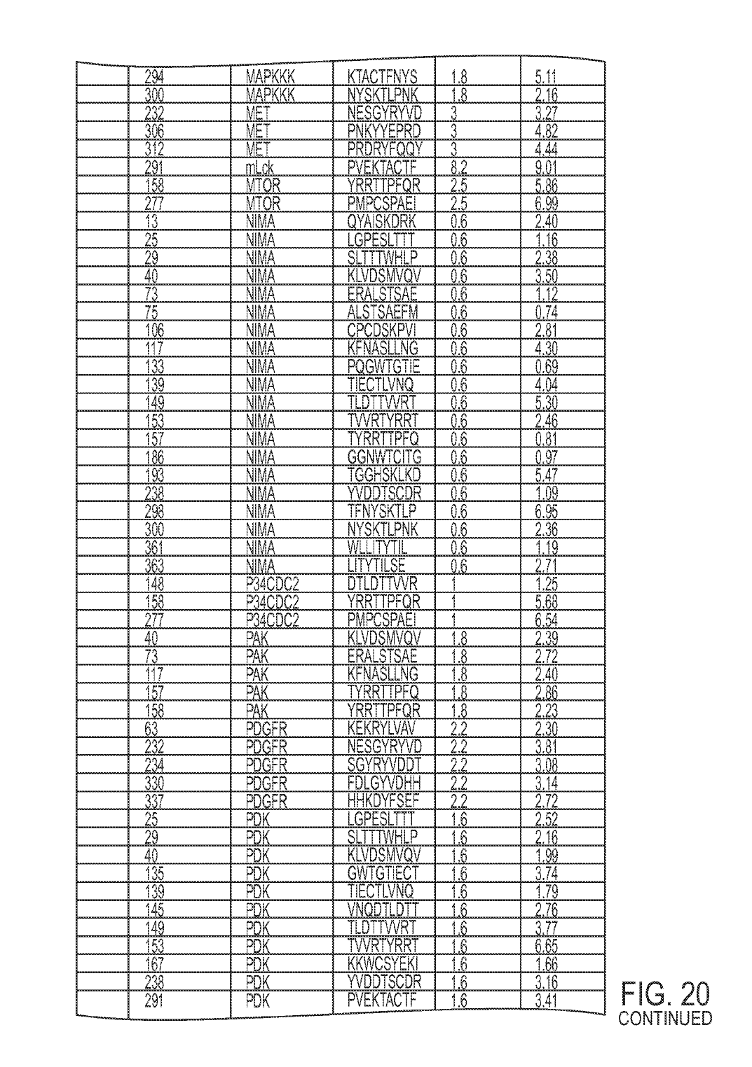

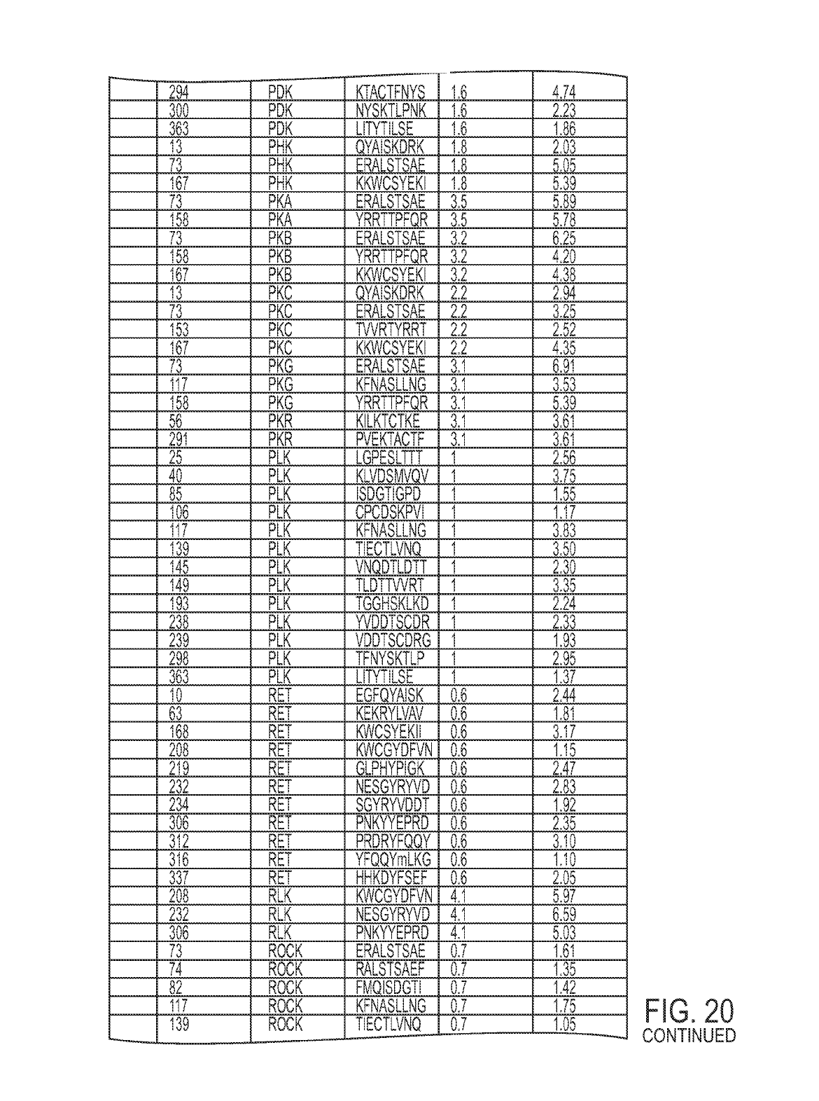

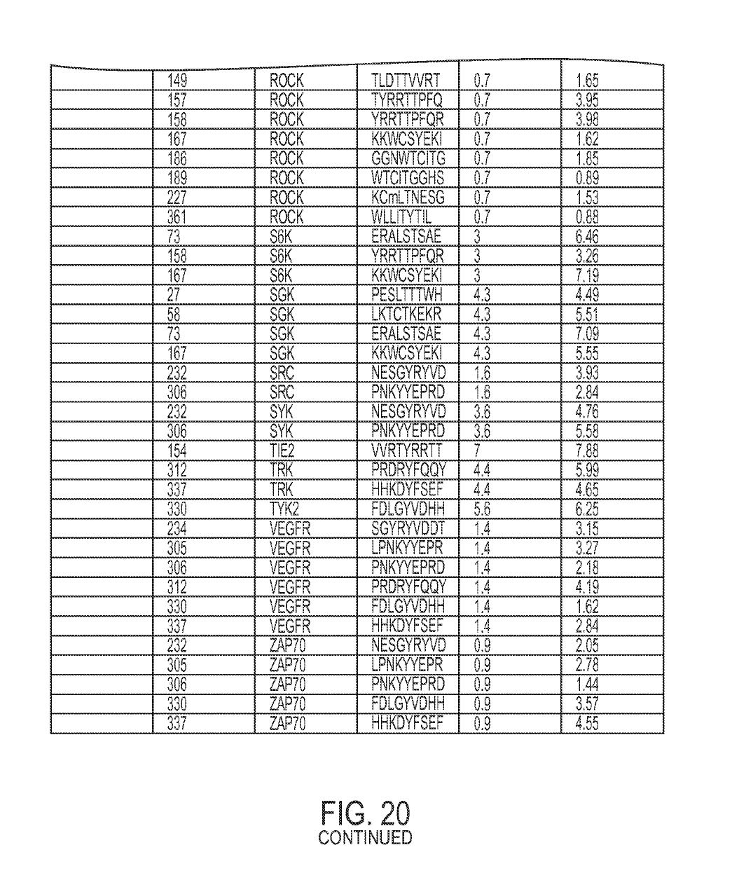

In certain embodiments, the HCV E2 peptide comprises at least about 10 residues of the HCV E2 protein and is 100 residues or less in length. Certain embodiments of the invention include various peptides and/or fusion proteins of HCV polypeptides, in particular HCV E2 protein. For example, all or part of a HCV E2 protein as set forth in FIGS. 19-20 may be used in various embodiments of the invention. In certain embodiments, a fragment of the HCV E2 may comprise, but is not limited to about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, 219, about 220 or more amino acid residues, and any range derivable therein.

It also will be understood that amino acid sequences may include additional residues, such as additional N- or C-terminal amino acids, and yet still be essentially as set forth in one of the sequences disclosed herein, so long as the sequence meets the criteria set forth above, including the maintenance of biological activity (e.g., immunogenicity) where protein expression is concerned. Theses non-envelope sequences may be termed “heterologous.”

A. Variants of Viral Envelope Polypeptides

Embodiments of the invention include various viral envelope polypeptides, peptides, and derivatives thereof. Amino acid sequence variants of a polypeptide can be substitutional, insertional or deletion variants. Deletion variants lack one or more residues of the native protein that are not essential for function or immunosuppressive activity. Insertional mutants typically involve the addition of material at a non-terminal point in the polypeptide. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. However, in particular, the present invention contemplates substitutional mutations that destroy one or more kinase sites within a viral envelope protein.

Conservative substitutions, designed to maintain function, are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of the HCV E2 polypeptides, provided the biological activity of the protein or peptide is maintained.

The term “functionally equivalent codon” is used herein to refer to codons that encode the same amino acid, such as the six codons for arginine or serine, and also refers to codons that encode biologically equivalent amino acids (see Table 1, below).

The following is a discussion based upon changing of the amino acids of a envelope polypeptide or peptide to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. The interactive capacity and nature of a protein that defines that protein's biological functional activity. However, certain amino acid substitutions can be made in a protein sequence, and in its underlying DNA or RNA coding sequence, and nevertheless produce a protein with like properties. It is thus contemplated by the inventors that various changes may be made in the DNA or RNA sequences of genes or coding regions without appreciable loss of their biological utility or activity, as discussed herein. Table 1 shows the codons that encode particular amino acids.

TABLE 1
CODON TABLE

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

It is understood that an amino acid substituted for another having a similar hydrophilicity value still produces a biologically equivalent and immunologically equivalent protein.

In certain embodiments, a envelope polypeptide may be a fusion protein. Fusion proteins may alter the characteristics of a given polypeptide, such antigenicity or purification characteristics. A fusion protein is a specialized type of insertional variant. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals, or transmembrane regions.

The present invention may employ peptides that comprise modified, non-natural and/or unusual amino acids. A table of exemplary, but not limiting, modified, non-natural and/or unusual amino acids is provided herein below. Chemical synthesis may be employed to incorporate such amino acids into the peptides of interest.

TABLE 2
Modified, Non-Natural and Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta-Amino- | Ahyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In addition to the variants discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides. Beta II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. These structures, which render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

The present invention may utilize an L-configuration amino acids, D-configuration amino acids, or a mixture thereof. While L-amino acids represent the vast majority of amino acids found in proteins, D-amino acids are found in some proteins produced by exotic sea-dwelling organisms, such as cone snails. They are also abundant components of the peptidoglycan cell walls of bacteria. D-serine may act as a neurotransmitter in the brain. The L and D convention for amino acid configuration refers not to the optical activity of the amino acid itself, but rather to the optical activity of the isomer of glyceraldehyde from which that amino acid can theoretically be synthesized (D-glyceraldehyde is dextrorotary; L-glyceraldehyde is levorotary).

One form of an “all-D” peptide is a retro-inverso peptide. Retro-inverso modification of naturally-occurring polypeptides involves the synthetic assemblage of amino acids with α-carbon stereochemistry opposite to that of the corresponding L-amino acids, i.e., D-amino acids in reverse order with respect to the native peptide sequence. A retro-inverso analogue thus has reversed termini and reversed direction of peptide bonds (NH—CO rather than CO—NH) while approximately maintaining the topology of the side chains as in the native peptide sequence. See U.S. Pat. No. 6,261,569, incorporated herein by reference.

B. In Vitro Production of Viral Envelope Polypeptides or Peptides

Various types of expression vectors are known in the art that can be used for the production of protein products. Following transfection with a expression vector, a cell in culture, e.g., a primary mammalian cell, a recombinant product may be prepared in various ways. A host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented (for exemplary methods see Freshney, 1992).

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large-scale production of cells and cell products. However, suspension cultured cells have limitations, such as tumorigenic potential and lower protein production than adherent cells.

In further aspects of the invention, other protein production methods known in the art may be used, including but not limited to prokaryotic, yeast, and other eukaryotic hosts such as insect cells and the like.

C. Protein Purification

It may be desirable to purify polypeptides and peptides, or variants and derivatives thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, hydrophobic interaction chromatography, exclusion chromatography; polyacrylamide gel electrophoresis; isoelectric focusing. A particularly efficient method of purifying peptides is fast protein liquid chromatography or even FPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “−fold purification number.” The actual units used to represent the amount of activity will, of course, be dependent upon the particular assay technique chosen to follow the purification and whether or not the expressed protein or peptide exhibits a detectable activity.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme.

D. Peptide Synthesis

Peptides may be generated synthetically for use in various embodiments of the present invention. Because of their relatively small size, the peptides of the invention can be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); Barany and Merrifield (1979), each incorporated herein by reference. Short peptide sequences, or libraries of overlapping peptides, usually from about 5 up to about 34 to 40 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

It may be desirable to purify polypeptide and peptides. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “−fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “−fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

### III. VIRAL POLYNUCLEOTIDES

Certain embodiments of the invention include viral envelope-coding polynucleotides or nucleic acid molecules and fragments thereof, as well as polynucleotides encoding envelope proteins of other viruses mentioned herein. The polynucleotides of the invention may be isolated and purified from other virus or cells infected or transfected with viral polynucleotides. The term isolated indicating they are free or substantially free from total viral or cellular genomic RNA or DNA, and proteins. It is contemplated that an isolated and purified virus nucleic acid molecule may take the form of RNA or DNA. A viral nucleic acid molecule refers to an RNA or DNA molecule that is capable of yielding all or part of a viral envelope protein from a transfected cell.

As used in this application, the term “polynucleotide” refers to a nucleic acid molecule, RNA, or DNA that has been isolated free of total genomic nucleic acid. Therefore, a “polynucleotide encoding all or part of an envelope protein” refers to a nucleic acid segment that contains envelope coding sequences, yet is isolated away from, or purified and free of, total viral genomic RNA and proteins; similarly, a “polynucleotide encoding full-length envelope” refers to a nucleic acid segment that contains full-length envelope coding sequences yet is isolated away from, or purified and free of, total viral genomic RNA and protein.

The term “cDNA” is intended to refer to DNA prepared using RNA as a template. The advantage of using a cDNA, as opposed to genomic RNA or an RNA transcript is stability and the ability to manipulate the sequence using recombinant DNA technology (See Maniatis, 1989; Ausubel, 1994). There may be times when the full or partial genomic sequence is preferred. Alternatively, cDNAs may be advantageous because it represents coding regions of a polypeptide and eliminates introns and other regulatory regions.

It also is contemplated that a given envelope may be represented by natural variants or strains that have slightly different nucleic acid sequences but, nonetheless, encode the same viral polypeptides (see Table 1 above). Consequently, the present invention also encompasses derivatives of envelope with minimal amino acid changes in its viral proteins, but that possesses the same activities. Also contemplated are envelope-encoding nucleic acids that encode modified envelope proteins lacking one or more kinase sites.

The term “gene” is used for simplicity to refer to the nucleic acid giving rise to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule may contain a contiguous envelope nucleic acid sequence of the following lengths: about 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more nucleotides, nucleosides, or base pairs.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated nucleic acid segments and recombinant vectors incorporating DNA sequences that encode viral envelope polypeptides or peptides that include within its amino acid sequence a contiguous amino acid sequence in accordance with, or essentially corresponding to viral envelope polypeptides. Also envisioned are variants that have modification in one or more kinase sites within these polypeptides.

The nucleic acid segments used in the present invention, regardless of the length of the coding sequence itself, may be combined with other DNA or RNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

D. Vectors Encoding HCV E2 or Other Viral Envelope Proteins

The present invention encompasses the use of vectors to encode for all or part of the envelope polypeptide. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). In particular embodiments, gene therapy or immunization vectors are contemplated. One of skill in the art would be well equipped to construct a vector through standard recombinant techniques, which are described in Maniatis et al., 1988 and Ausubel et al., 1994, both incorporated herein by reference.

The term “expression vector” or “expression construct” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” means that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the nucleic acid segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or exogenous, i.e., from a different source than viral sequence. In some examples, a prokaryotic promoter is employed for use with in vitro transcription of a desired sequence. Prokaryotic promoters for use with many commercially available systems include T7, T3, and Sp6.

Table 3 lists several elements/promoters that may be employed, in the context of the present invention, to regulate the expression of a gene. This list is not intended to be exhaustive of all the possible elements involved in the promotion of expression but, merely, to be exemplary thereof. Table 4 provides examples of inducible elements, which are regions of a nucleic acid sequence that can be activated in response to a specific stimulus.

TABLE 3
Promoter and/or Enhancer

| Promoter/Enhancer | References |
|---|---|
| Immunoglobulin Heavy Chain | Banerji et al., 1983; Gilles et al., 1983; |
| | Grosschedl et al., 1985; Atchinson et al., 1986, |
| | 1987; Imler et al., 1987; Weinberger et al., |
| | 1984; Kiledjian et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; |
| | Johnson et al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh |
| | et al., 1985; Firak et al., 1986; Herr et al., 1986; |
| | Imbra et al., 1986; Kadesch et al., 1986; Wang |
| | et al., 1986; Ondek et al., 1987; Kuhl et al., |
| | 1987; Schaffner et al., 1988 |
| Polyoma | Swartzendruber et al., 1975; Vasseur et al., |
| | 1980; Katinka et al., 1980, 1981; Tyndell et al., |
| | 1981; Dandolo et al., 1983; de Villiers et al., |
| | 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Chol et al., 1988; |
| | Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987; Glue et al., 1988 |
| Hepatitis B Virus | Bulla et al., 1986; Jameel et al., 1986; Shaul et |
| | al., 1987; Spandau et al., 1988; Vannice et al., |
| | 1988 |
| Human Immunodeficiency Virus | Muesing et al., 1987; Hauber et al., 1988; |
| | Jakobovits et al., 1988; Feng et al., 1988; |
| | Takebe et al., 1988; Rosen et al., 1988; |
| | Berkhout et al., 1989; Laspia et al., 1989; Sharp |
| | et al., 1989; Braddock et al., 1989 |
| Cytomegalovirus (CMV) | Weber et al., 1984; Boshart et al., 1985; |
| | Foecking et al., 1986 |
| Gibbon Ape Leukemia Virus | Holbrook et al., 1987; Quinn et al., 1989 |

TABLE 4
Inducible Elements

| Element | Inducer | References |
|---|---|---|
| MT II | Phorbol Ester (TFA) | Palmiter et al., 1982; |
| | Heavy metals | Haslinger et al., 1985; Searle |
| | | et al., 1985; Stuart et al., |
| | | 1985; Imagawa et al., 1987, |
| | | Karin et al., 1987; Angel et |
| | | al., 1987b; McNeall et al., |
| | | 1989 |
| MMTV (mouse mammary | Glucocorticoids | Huang et al., 1981; Lee et |
| tumor virus) | | al., 1981; Majors et al., |
| | | 1983; Chandler et al., 1983; |
| | | Lee et al., 1984; Ponta et al., |
| | | 1985; Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee et al., 1997), insulin-like growth factor II (Wu et al., 1997), human platelet endothelial cell adhesion molecule-1 (Almendro et al., 1996).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. See Chandler et al., 1997, herein incorporated by reference.

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

For expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

8. Selectable and Screenable Markers

In certain embodiments of the invention, the cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

E. Host Cells

As used herein, the terms “cell,” “cell line,” and “cell culture” may be used interchangeably. All of these terms also include their progeny, which refers to any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector, expression of part or all of the vector-encoded nucleic acid sequences, or production of infectious viral particles. Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either an eukaryotic or prokaryotic host cell, particularly one that is permissive for replication or expression of the vector.

F. Expression Systems

Numerous expression systems exist that comprise at least all or part of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM from CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. The Tet-On™ and Tet-Off™ systems from CLONTECH® can be used to regulate expression in a mammalian host using tetracycline or its derivatives. The implementation of these systems is described in Gossen et al. (1992) and Gossen et al. (1995), and U.S. Pat. No. 5,650,298, all of which are incorporated by reference.

INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

G. Introduction of Nucleic Acids into Cells

In certain embodiments, a nucleic acid may be introduce into a cell in vitro for production of polypeptides or in vivo for immunization purposes. There are a number of ways in which nucleic acid molecules such as expression vectors may be introduced into cells. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986).

“Viral expression vector” is meant to include those vectors containing sequences of that virus sufficient to (a) support packaging of the vector and (b) to express a polynucleotide that has been cloned therein. In this context, expression may require that the gene product be synthesized. A number of such viral vectors have already been thoroughly researched, including adenovirus, adeno-associated viruses, retroviruses, herpesviruses, and vaccinia viruses.

Delivery may be accomplished in vitro, as in laboratory procedures for transforming cells lines, or in vivo or ex vivo, as in the treatment of certain disease states. One mechanism for delivery is via viral infection where the expression vector is encapsidated in an infectious viral particle. Several non-viral methods for the transfer of expression vectors into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), liposome (Ghosh and Bachhawat, 1991; Kaneda et al., 1989) and receptor-mediated transfection (Wu and Wu, 1987; Wu and Wu, 1988). Some of these techniques may be successfully adapted for in vivo or ex vivo use.

In certain embodiments, the nucleic acid encoding a gene or genes may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression vector is delivered to a cell and where in the cell the nucleic acid remains is dependent on the type of expression vector employed.

Transfer of a nucleic acid molecule may be performed by any of the methods mentioned above which physically or chemically permeabilize the cell membrane. This is particularly applicable for transfer in vitro, but it may be applied to in vivo use as well.

### IV. IMMUNOSUPPRESSIVE THERAPY

A. Inflammatory Conditions

The present invention relates to the use of viral compositions (polypeptides, peptides, nucleic acids coding therefor, and mimetics) for the modulation of immune responses, particularly those relating to pathologic inflammation. In one embodiment, the pathologic inflammation relates to interleukin-2 (IL-2) expression. IL-2 has multiple, sometimes opposing, functions during an inflammatory response. It is a potent inducer of T cell proliferation and T-helper 1 (Th1) and Th2 effector T cell differentiation and provides T cells with a long-lasting competitive advantage resulting in the optimal survival and function of memory cells. In a regulatory role, IL-2 is important for the development, survival, and function of regulatory T cells, it enhances Fas-mediated activation-induced cell death, and it inhibits the development of inflammatory Th17 cells. Thus, in its dual and contrasting functions, IL-2 contributes to both the induction and the termination of inflammatory immune responses.

The present invention would therefore seek to intervene in those disease where, for example, IL-2 is activating T cells and leading to inflammatory states. Such diseases include autoimmune diseases like multiple sclerosis, psoriasis, inflammatory bowel disorders, early arthritis, juvenile arthritis, rheumatoid arthritis, enteropathic arthritis, psoriatic arthritis, ankylosing spondylitis, familial Mediterranean fever, amyotrophic lateral sclerosis, systemic lupus erythematosus, ulcerative colitis, inflammatory bowel disease, Sjögren's syndrome, or Crohn's disease. Other inflammatory conditions include cardiovascular disease, trauma, or pancreatitis.

B. Combinations with Anti-Inflammatories

It is common in many fields of medicine to treat a disease with multiple therapeutic modalities, often called “combination therapies.” Inflammatory diseases are no exception. To treat inflammatory disorders using the methods and compositions of the present invention, one would generally contact a target cell or subject with a viral immunosuppressive segment and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes a viral immunosuppressive segment and the other includes the other agent.

Alternatively, the immunosuppressive viral peptide or polypeptide may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either a viral immunosuppressive segment or the other therapy will be desired. Various combinations may be employed, where the a viral immunosuppressive segment is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated.

Agents or factors suitable for use in a combined therapy against an inflammatory disorder include steroids, glucocorticoids, non-steriodal anti-inflammatory drugs (NSAIDS; including COX-1 and COX-2 inhibitors), aspirin, ibuprofen, and naproxen. Analgesics are commonly associated with anti-inflammatory drugs but which have no anti-inflammatory effects. An example is paracetamol, called acetaminophen in the U.S. and sold under the brand name of Tylenol. As opposed to NSAIDS, which reduce pain and inflammation by inhibiting COX enzymes, paracetamol has recently been shown to block the reuptake of endocannabinoids, which only reduces pain, likely explaining why it has minimal effect on inflammation.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating inflammation.

### V. PHARMACEUTICAL COMPOSITIONS AND ROUTES OF ADMINISTRATION

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render proteins stable. Buffers also will be employed when proteins are introduced into a patient. Aqueous compositions of the present invention comprise an effective amount of the protein or polypeptide, dissolved or dispersed in a pharmaceutically acceptable carrier or aqueous media. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The percentage of active compound in any pharmaceutical preparation is dependent upon both the activity of the compound. Typically, such compositions should contain at least 0.1% active compound. The percentage of the compositions and preparations may, of course, be varied and may conveniently be between about 2 to about 60% of the weight of the unit. The amount of active compounds in such therapeutically useful compositions is such that a suitable dosage will be obtained.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy injection is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, phenylmecuric nitrate, m-cresol, and the like. In many cases, it will be preferable to use isotonic solutions, for example, sugars or sodium chloride. Prolonged absorption of the injectable compositions can be brought about by the use in the compositions of agents delaying absorption, for example, aluminum monostearate, and gelatin.

Sterile injectable solutions are prepared by incorporating the active compounds in the required amount in the appropriate solvent with various of the other ingredients enumerated above, as required, followed by sterile filtration. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques that yield a powder of the active ingredient plus any additional desired ingredient from a previously sterile-filtered solution thereof

The present invention contemplates a viral immunosuppressive segment, and nucleic acid molecules coding therefor. In some embodiments, pharmaceutical compositions are administered to a subject. Different aspects of the present invention involve administering an effective amount of an aqueous composition. Such compositions will generally be dissolved or dispersed in a pharmaceutically acceptable carrier or aqueous medium. Those of skill in the art are well aware of how to apply antibodies or other binding agents, as well as gene delivery to in vivo and ex vivo situations.

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including cremes, lotions, mouthwashes, inhalants and the like.

The active compounds of the present invention can be formulated for parenteral administration, e.g., formulated for injection via the intravenous, intramuscular, intrathoracic, sub-cutaneous, or even intraperitoneal routes. Administration by i.v. or i.m. is specifically contemplated.

The active compositions may be formulated as neutral or salt forms. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In certain embodiments, it may be desirable to provide a continuous supply of compositions to the patient. For intravenous or intraarterial routes, this is accomplished by drip system. For various approaches, delayed release formulations could be used that provided limited but constant amounts of the therapeutic agent over and extended period of time. For internal application, continuous perfusion, for example with a HCV peptide, may be preferred. This could be accomplished by catheterization followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered. It is believed that higher doses may be achieved via perfusion, however.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, Remington's Pharmaceutical Sciences, 1990). Some variation in dosage will necessarily occur depending on the condition of the subject being treated. The person responsible for administration will, in any event, determine the appropriate dose for the individual subject.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Peptides may be administered in a dose that can vary from 0.01, 0.05, 0.1, 0.5, 1, 5, 10, 20, 30, 40, 50, 60, 70, 80, 90, 100 mg/kg of weight to 50, 60, 70, 80, 90, 100, 110, 120, 130, 140, 150, 160, 170, 180, 190, 200 mg/kg of weight in one or more daily, weekly, monthly, or yearly administrations during one or various days, weeks, months, or years. The proteins or peptides can be administered by parenteral injection (intravenous, intraperitoneal, intramuscular, subcutaneous, intracavity or transdermic).

In many instances, it will be desirable to have multiple administrations of the peptides or other compositions of the invention. The compositions of the invention may be administered 1, 2, 3, 4, 5, 6, 7, 8, 9, 10, or more times. The administrations will normally be at from one to twelve week intervals, more usually from one to four week intervals.

Dosages commonly used for formulations that provide passive immunity are in the range of from 0.5 ml to 10 ml per dose, preferably in the range of 2 ml to 5 ml per dose. Repeated doses to deliver the appropriate amount of active compound are common Both the age and size by weight of the recipient must be considered when determining the appropriate dosage of active ingredient and volume to administer.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability, and toxicity of the particular therapeutic substance.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

As used herein, the term in vitro administration refers to manipulations performed on cells removed from an animal, including, but not limited to, cells in culture. The term ex vivo administration refers to cells that have been manipulated in vitro, and are subsequently administered to a living animal. The term in vivo administration includes all manipulations performed on cells within an animal

### VI. VACCINES

In an embodiment of the present invention, a method of inducing an enhanced immune response to the engineered viral proteins rather than native proteins prevent or limit viral infection is provided. Modified viral envelope proteins lacking one or more kinase sites will be used in subunit or whole virus immunization. An effective amount of a vaccine composition, generally, is defined as that amount sufficient to detectably and repeatedly ameliorate, reduce, minimize or limit the extent of the disease or condition or symptoms thereof. More rigorous definitions may apply, including elimination, eradication or cure of disease.

A. Administration

The compositions of the present invention may be used in vivo to produce anti-virus immune response, and thus constitute therapeutic and prophylactic vaccines. Thus, the compositions can be formulated for parenteral administration, e.g., formulated for injection via the intradermal, intravenous, intramuscular, subcutaneous, or intraperitoneal routes. Administration by intradermal and intramuscular routes is specifically contemplated. The vaccine can also be administered by a topical route directly to the mucosa, for example by nasal drops or mist, inhalation, or by nebulizer.

Some variation in dosage and regimen will necessarily occur depending on the age and medical condition of the subject being treated, as well as the route chosen. The person responsible for administration will, in any event, determine the appropriate dose for the individual subject. In many instances, it will be desirable to have multiple administrations of the vaccine. Thus, the compositions of the invention may be administered 1, 2, 3, 4, 5, 6, 7, 8, 9, 10, or more times. The administrations will normally be at from one to twelve week intervals, more usually from one to six week intervals. Periodic re-administration will be desirable with recurrent exposure to the pathogen.

The administration may use various “unit doses.” Unit dose is defined as containing a predetermined-quantity of the therapeutic composition. The quantity to be administered, and the particular route and formulation, are within the skill of those in the clinical arts.

B. Measuring Immune Responses

One of ordinary skill would know various assays to determine whether an immune response against a vaccine was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al. (1991), hereby incorporated by reference.

C. Injectable Formulations

One method for the delivery of a pharmaceutical according to the present invention is via injection. However, the pharmaceutical compositions disclosed herein may alternatively be administered intravenously, intradermally, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety).

Injection may be by syringe or any other method used for injection of a solution, as long as the agent can pass through the particular gauge of needle required for injection. A novel needleless injection system has been described (U.S. Pat. No. 5,846,233) having a nozzle defining an ampule chamber for holding the solution and an energy device for pushing the solution out of the nozzle to the site of delivery.

Solutions of the vaccine as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile aqueous media that can be employed will be known to those of skill in the art in light of the present disclosure. For example, one dosage may be dissolved in 1 ml of isotonic NaCl solution and either added to 1000 ml of hypodermolysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous injectable composition that contains a protein as an active ingredient is well understood in the art.

D. Inhalable or Aerosol Formulations

A particular mode of administration contemplated by the inventor for the peptides of the present invention is via inhalation and/or administration to the nasal mucosa, i.e., intranasal administration. A variety of commercial vaccines (influenza, measles) are currently administered using a nasal mist formulation. The methods of the present invention can be carried out using a delivery similar to that used with the Flu-Mist® product, which employs the BD AccuSpray® System (Becton Dickinson). Also useful for this route are nebulizers, such as jet nebulizers and ultrasonic nebulizers.

E. Additional Vaccine Components

In other embodiments of the invention, the antigenic composition may comprise an additional immunostimulatory agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination.

i. Adjuvants

As also well known in the art, the immunogenicity of a particular immunogen composition can be enhanced by the use of non-specific stimulators of the immune response, known as adjuvants. Adjuvants have been used experimentally to promote a generalized increase in immunity against unknown antigens (e.g., U.S. Pat. No. 4,877,611) Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. Suitable molecule adjuvants include all acceptable immunostimulatory compounds, such as cytokines, toxins or synthetic compositions.

Exemplary, often preferred adjuvants include complete Freund's adjuvant (a non-specific stimulator of the immune response containing killed Mycobacterium tuberculosis), incomplete Freund's adjuvants and aluminum hydroxide adjuvant. Other adjuvants that may also be used include IL-1, IL-2, IL-4, IL-7, IL-12, α-interferon, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopol®) used as an about 0.25% solution. Adjuvant effect may also be made my aggregation of the antigen in the vaccine by heat treatment with temperatures ranging between about 70° to about 101° C. for a 30-second to 2-minute period, respectively. Aggregation by reactivating with pepsin-treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

Some adjuvants, for example, certain organic molecules obtained from bacteria, act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. It is effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present invention.

BCG (bacillus Calmette-Guerin, an attenuated strain of Mycobacterium) and BCG-cell wall skeleton (CWS) may also be used as adjuvants, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945. BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martin et al., 1990). Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface active agents, e.g., saponin and derivatives such as QS21 (Cambridge Biotech), form yet another group of adjuvants for use with the immunogens of the present invention. Nonionic block copolymer surfactants (Rabinovich et al., 1994) may also be employed. Oligonucleotides are another useful group of adjuvants (Yamamoto et al., 1988). Quil A and lentinen are other adjuvants that may be used in certain embodiments of the present invention.

Another group of adjuvants are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the invention (Takada et al., 1995).

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

Adjuvants may be encoded by a nucleic acid (e.g., DNA or RNA). It is contemplated that such adjuvants may be also be encoded in a nucleic acid (e.g., an expression vector) encoding the antigen, or in a separate vector or other construct. Nucleic acids encoding the adjuvants can be delivered directly, such as for example with lipids or liposomes.

ii. Biological Response Modifiers

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as α-interferon, IL-2, or IL-12 or genes encoding proteins involved in immune helper functions, such as B-7.

iii. Chemokines

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-α, MIP1-β, IP-10 and combinations thereof. The skilled artisan will recognize that certain cytokines (e.g., IFN's) are also known to have chemoattractant effects and could also be classified under the term chemokines.

### VII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Expression of GBV-C E2 Protein.

Tet-off Jurkat cell lines expressing GBV-C E2 protein (nt 1167-2161 based on GenBank AF 121950), the vector control (expressing GFP) and E2 coding sequence with a plus one frameshift mutation inserted to abolish protein expression (FS control) were previously described (Bhattarai et al., 2012a). Six truncated E2 proteins were ligated into a modified pTRE2-HGY plasmid (Clontech, Inc.) as described (Xiang et al., 2012). This plasmid generates a bicistronic message encoding the GBV-C E2 sequence followed by the encephalomyocarditis virus (EMC) internal ribosomal entry site (IRES) that directs translation of GFP. Jurkat (tet-off) cell lines (Clontech, Inc) were transfected (Nucleofector II, Lonza Inc.) and cell lines selected for resistance to hygromycin and neomycin. GFP positive cells were bulk sorted using a BD FACS Diva (University of Iowa Flow Cytometry Facility). Protein expression was analyzed by measuring GFP by flow cytometry (BD LSR II) and by immunoblot using antibodies directed against a C-terminal histidine tag (Qiagen). All cell lines were maintained in RPMI 1640 supplemented with 10% heat-inactivated fetal calf serum, 2 mM L-glutamine, 100 IU/ml penicillin, and 100 μg/ml streptomycin with hygromycin and neomycin (200 μg/ml). Insert sequences were confirmed by sequencing plasmid DNA (University of Iowa DNA Core Facility).

Cell Stimulation.

Jurkat cells (5×10⁶ cells/ml) were stimulated with plate-bound anti-CD3 (5 μg/ml, OKT3 clone, eBioscience) and soluble CD28 antibody (5 μg/ml, clone CD28.2, BD Biosciences) unless stated otherwise. For co-culture experiments, non-transfected GFP negative Jurkat cells (5×10⁵ cells/ml) were incubated with either the transfected GFP positive vector control or GFP positive GBV-C E2 expressing cells (1×10⁶ cells/ml) for 72 hours prior to stimulation with anti-CD3/CD28. Following 24 hours of stimulation, cellular receptor expression and cytokine release were measured by flow cytometry in GFP negative cells and by ELISA respectively.

Flow Cytometry.

Cellular receptor expression was measured with CD69 (PE), CD25 (APC), or CD45 (PE) (BD Biosciences) using the manufacturer's recommendation. Cells were incubated on ice for 1 hour, washed 3 times with PBS and fixed in 2% paraformaldehyde (Polysciences). Data was acquired on BD LSR II flow cytometer using single stained CompBeads (BD Biosciences) for compensation. At least 10,000 total events were collected in each experiment and the FlowJo program (Tree Star Inc.) was used for data analysis. All flow cytometry experiments were repeated at least three times with consistent results.

Immunoblot Analysis.

Jurkat cells (5×10⁶) were stimulated with anti-CD3 (5 μg/ml) for the time indicated prior to addition of cell lysis buffer (Cell Signaling) for 15 minutes and sonication. Lysates were separated by polyacrylamide gel electrophoresis and transferred to nitrocellulose membranes (BIORAD). Membranes were incubated in protein-free blocking buffer (Thermo Scientific) for 1 hour at room temperature followed by incubation with primary antibodies Immunoreactive proteins were detected with Amersham ECL (GE Healthcare) using a Kodak Imager. Protein phosphorylation was quantified using ImageJ (NIH) and normalized to total protein levels. Primary antibodies used were: pLAT (Y226; BD Biosciences); total LAT (Biolegend); CD63 antibodies (Systems Biosciences); and pZAP70 (Y319); total ZAP70; pLck (Y505); pLck (Y394/pSrcY416); total Lck (Y394) and total Csk (all from Cell Signaling Technology). For immune precipitation studies, Jurkat cell lysates were incubated with recombinant Fc fused GBV-C E2 protein (Bhattarai et al., 2012a), or GBV-C E2 expressing Jurkat cell lysates were incubated with anti-Lck antibodies overnight at 4° C. as described. Protein complexes were isolated from the cellular lysates using protein A/G agarose beads (Thermo Scientific) and precipitated proteins were detected by immunoblot analysis.

ELISA.

pLAT (Y191) was quantified using PathScan ELISA kit (Cell Signaling Technology) and IL-2 cytokine released into cell culture supernatant was quantified using human IL-2 quantikine ELISA kit (R&D Systems) according to manufacturer's instructions.

Enzyme Activity Assays.

CD45 activity was measured using CD45 tyrosine phosphatase assay kit (Enzo Life Sciences) following the manufacturer's instructions. Purified recombinant GBV-C E2 protein expressed in CHO cells was described previously (Bhattarai et al., 2012a). Enzymatic activity was evaluated with or without GBV-C E2 protein (10 μg) or human IgG control (10 μg; Sigma) at room temperature. Following 1-hour incubation, the reaction was terminated and absorbance determined by a Microplate reader (Model 680, Bio-Rad) at OD620 nm. Phosphorylation of GBV-C E2 protein by Lck was measured by incubating recombinant E2 protein (40 μg) with or without human Lck (500 ng; R&D Systems) as recommended by the manufacturer. Samples were subjected to immunoblot analysis as described above. Phosphorylation was determined by immunoblot analysis with phosphotyrosine antibodies (Invitrogen) and GBV-C E2 protein was identified using an anti-E2 monoclonal antibody as described (Mohr et al., 2010). Lck mediated phosphorylation of GBV-C E2 derived TAT peptides were performed using Lck kinase enzyme system (Promega) as recommended by the manufacturer.

GBV-C E2 Synthetic Peptides.

FITC labelled synthetic peptides with an N-terminal HIV TAT protein transduction domain (TAT) alone (GGGGGRKKRRQRRR), or with the GBV-C E2 aa 86-101 (GGGGGRKKRRQRRRVYGSVSVTCVWGS; Y87), or the Y87H mutation (GGGGGRKKRRQRRRVHGSVSVTCVWGS) were purchased from Ana Spec, Inc. Peptides with the TAT domain and GBV-C E2 aa 276-292 (GGAGLTGGRYEPLVRRC), or the same amino acids in a scrambled order (GCRCARGVLLTPGEGYF) were previously described (Xiang et al., 2012). Peptides were dissolved in RPMI with 10% DMSO. Healthy donor PBMCs (1×10⁶ cells/ml) were incubated with 20 μg peptide at 37° C. over night before stimulation with 500 ng/ml anti-CD3/CD28. IL-2 release and cellular receptor expression was analyzed 24 hrs later.

GBV-C RNA Quantification.

GBV-C viremic HIV-infected subjects receiving cART who were attending the University of Iowa HIV Clinic and healthy volunteer blood donors were invited to participate. HIV-infected subjects' HIV viral load (VL) was below the limit of detection (<48 copies/mL) for a minimum of 6 months and at the time of blood donation. All subjects and healthy blood donors provided written informed consent, and the study was approved by the University of Iowa Institutional Review Board. PBMCs were prepared as described (Rydze et al., 2012). For sorting experiments, CD3+ T cells were enriched using Automacs (Miltenyi Biotech), and CD3+ T cells were sorted into CD4+ and CD8+ populations by FACS (BD ARIA II) using CD3 (V450), CD4 (FITC), CD8 (Alexa700) antibodies (all BD Biosciences). Sorted cells were counted using Countess™ automated cell counter (Invitrogen). Total cellular RNA from specific T cell populations was isolated and GBV-C RNA was quantified by real-time RT-PCR as described (Rydze et al., 2012).

Extracellular Microvesicles (EMV) Isolation.

EMV were purified from the clarified cell-culture supernatant or from human serum using the ExoQuick reagent (Systems Biosciences) according to the manufacturer's instructions. This commercial reagent has been previously reported to yield EMV from cell culture supernatant and human serum (Bala et al., 2012; Fabbri et al., 2012; Singh et al., 2012; Zhuang et al., 2012). Sodium chloride (NaCl) density flotation was performed as described (Xiang et al., 1998). Briefly, 1 ml of undiluted serum was mixed with 35 ml of NaCl solution (1.063 g/ml), and centrifuged in a Beckman SW28 rotor (112,000×g, 4° C.×65 hrs). Following centrifugation, fractions were collected for subsequent analysis. PBMCs from healthy donors were incubated with EMV purified from 5 ml of GBV-C positive or GBV-C negative serum or EMV purified from 10 ml of culture supernatant overnight and stimulated with anti-CD3/CD28 antibodies (500 ng/ml) for 24 hours before analysis. Statistics: Statistics were performed using GraphPad software V4.0 (GraphPad Software Inc.). Two-sided Student's t test was used to compare results between GBV-C E2 protein expressing cells and controls. P values less than 0.05 were considered statistically significant.

YFV Studies.

YFV replication in Lck-deficient and rescued (Lck+) Jurkat cells, primary human T cells and murine CD3+ enriched splenocytes was measured by YFV RNA or TCID₅₀ (BHK-21 cells). TCR activation was measured by IL-2 release (ELISA) and CD69 expression (FACS) following anti-CD3/CD28 or PMA-ionomycin (P-I) stimulation. Jurkat cells stably expressing YFV env and env deletion mutants were studied.

### Example 2

### Results

Extracellular Microvesicles from GBV-C Infected Human Serum Inhibit T Cell Receptor (TCR) Signaling in Primary Human T Cells.

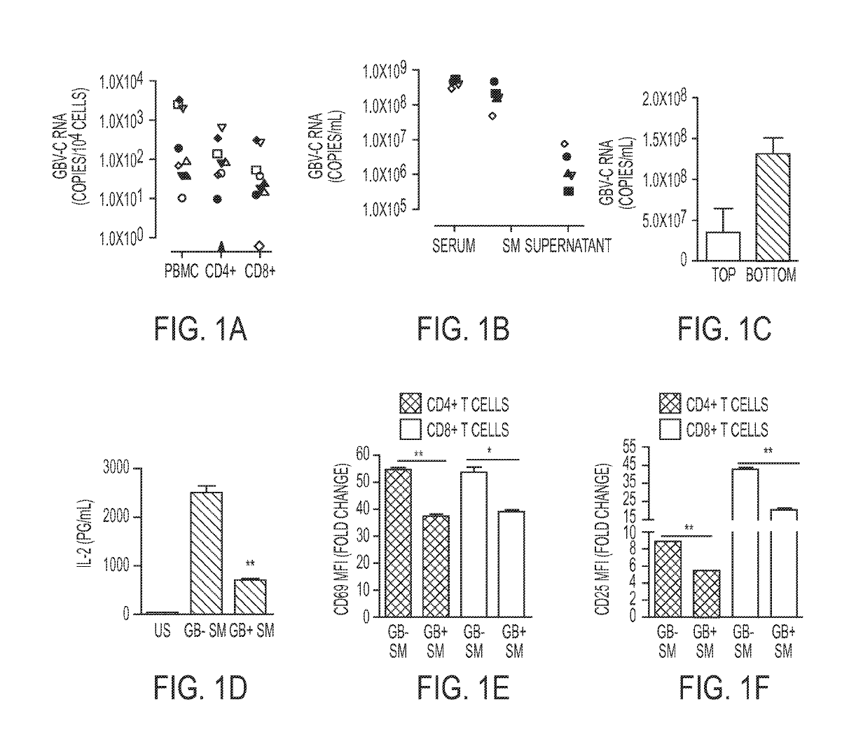

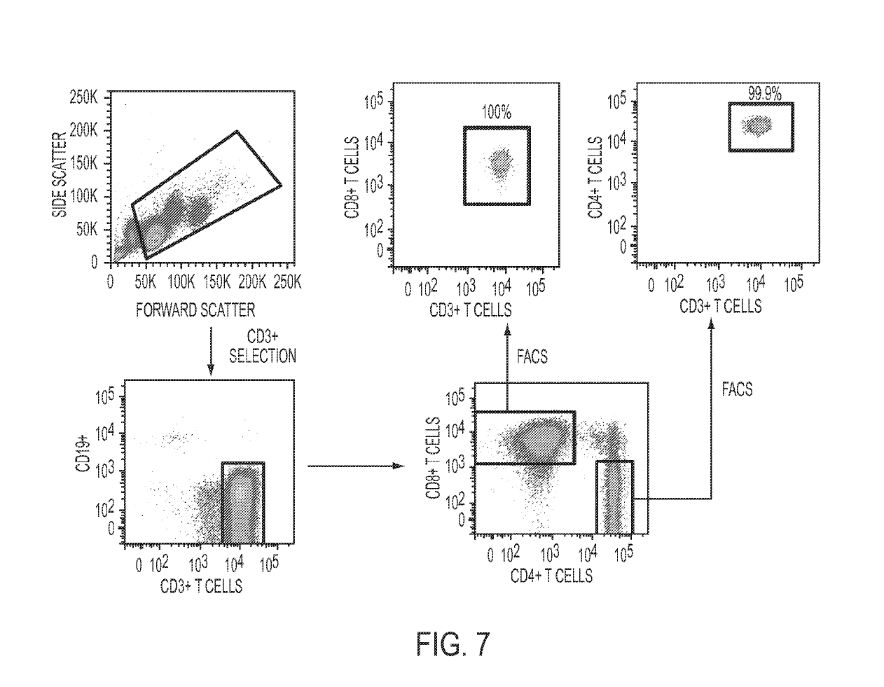

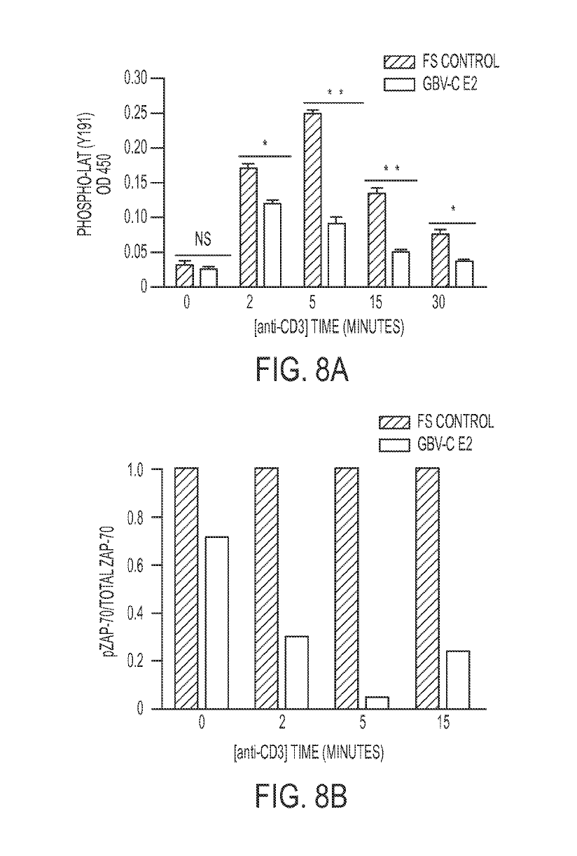

GBV-C infection is associated with global reduction in T cell activation and reduced IL-2 signaling in peripheral blood mononuclear cells (PBMCs) (Bhattarai et al., 2012b; Maidana-Giret et al., 2009; Rydze et al., 2012; Stapleton et al., 2012; Stapleton et al., 2009). Since the frequency of GBV-C infected lymphocytes in peripheral blood is unknown, GBV-C RNA copy number within CD4+ and CD8+ T cells obtained from nine GBV-C viremic subjects was determined Using immunoaffinity selection and fluorescent activated cell sorting (FACS), highly purified (>99%) CD4+ and CD8+ T cells were recovered from peripheral blood mononuclear cells (PBMCs) (FIG. 7). GBV-C RNA was detected in PBMCs obtained from all nine subjects with an average of 879 genome equivalents (G.E.) per 10⁴ cells (FIG. 1A). Viral RNA was detected in both CD4+ T cells (average 146 GE per 10⁴ cells) and CD8+ T cells (average 77 GE per 10⁴ cells) in all but two subjects. One of these subjects had GBV-C RNA detected in CD4+ T cells while the other had GBV-C RNA present in only the CD8+ T cell population (FIG. 1A). If only one copy of GBV-C RNA is produced per cell, then less than 10% of PBMCs are infected. It is likely that infected cells contain multiple copies of viral RNA and thus the proportion of GBV-C infected PBMCs is much lower than 10%. Since clinical studies demonstrate global reduction in CD4+ and CD8+ T cell activation in GBV-C infected subjects (Bhattarai et al., 2012b; Maidana-Giret et al., 2009; Stapleton et al., 2012), GBV-C infection must alter T cell activation in uninfected T cells.

The closely related hepatitis C virus (HCV) transmits viral RNA and proteins to bystander cells via extracellular microvesicles (Dreux et al., 2012; Masciopinto et al., 2004). The related GBV-C may employ a similar mechanism to interact with uninfected bystander cells. To test this hypothesis, extracellular microvesicles (EMV) purified from the sera of GBV-C viremic subjects were examined for GBV-C RNA. Serum EMV were purified using a commercial reagent (Exoquick), and more than 98% of serum GBV-C RNA was precipitated (FIG. 1B). Consistent with a previous study (Xiang et al., 1998), saline flotation gradient centrifugation of GBV-C-positive serum yielded two populations of GBV-C RNA-containing particles with distinctly different densities. Viral RNA was concentrated in the low-density fraction (1.07 g/ml; FIG. 1C, Top), consistent with virions associated with LDL, and in a heavier fraction (˜1.16 g/ml; FIG. 1C, Bottom). The density of the heavier particles was similar to that described for vesicles of endocytic origin (exosomes; 1.10-1.19 g/ml) (Meckes and Raab-Traub, 2011) and were precipitated by the commercial exosome purification reagent (Exoquick). In contrast, the low density particles did not precipitate with Exoquick reagent, suggesting microvesicles of endocytic origin are preferentially precipitated by Exoquick reagent (data not shown). Primary human CD4+ and CD8+ T cells from healthy blood donors were incubated with EMV prepared from GBV-C viremic (GB+ EMV) or GBV-C nonviremic (GB−EMV) sera prior to TCR engagement with CD3/CD28 antibodies. GB+ EMV inhibited TCR-mediated signaling compared to GB− EMV as measured by the release of IL-2 into culture supernatants (FIG. 1D), or by cell surface expression of T cell activation markers (CD69 and CD25; FIGS. 1E-F). These data demonstrate that GBV-C RNA containing microvesicles in the serum of infected subjects inhibited TCR signaling in uninfected T cells, providing a potential mechanism to explain the global reduction in T cell activation observed in humans with HIV-GBV-C coinfection.

GBV-C E2 Protein Inhibits TCR-Mediated Activation of CD4+ T Cells.

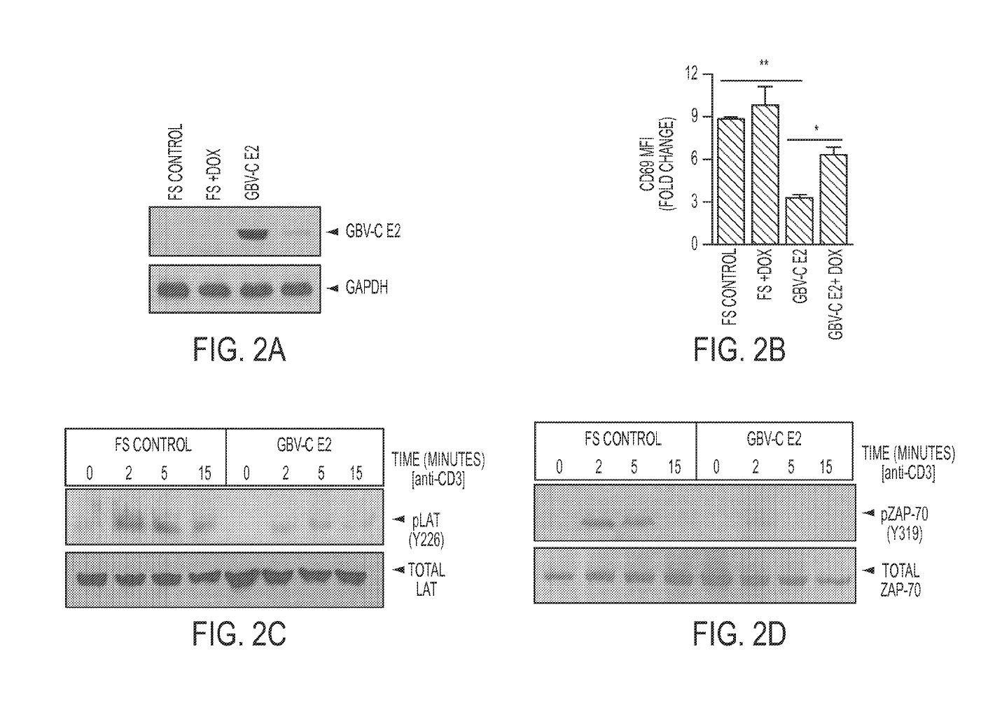

The GBV-C envelope glycoprotein E2 was previously shown to inhibit activation and IL-2 signaling pathways in human T cells (Bhattarai et al., 2012a). To determine if TCR activation was altered by E2 protein, E2 RNA or both, activation was measured in tet-off Jurkat (CD4+) T cells before and following TCR stimulation with CD3/CD28 antibodies. Tet-off Jurkat cells stably expressing GBV-C E2 protein or the GBV-C E2 coding sequence in which a plus one frame shift (FS) was inserted to abolish translation were incubated with or without doxycycline (1 μg/ml) for 5 days to reduce transcription of the GBV-C E2 sequence (FIG. 2A). Activation following TCR stimulation, as measured by CD69 expression, was significantly inhibited in E2 expressing Jurkat cells compared to the control FS cells expressing the E2 RNA (FIG. 2B). Inhibition was significantly reversed in cells maintained in doxycycline (FIG. 2B).

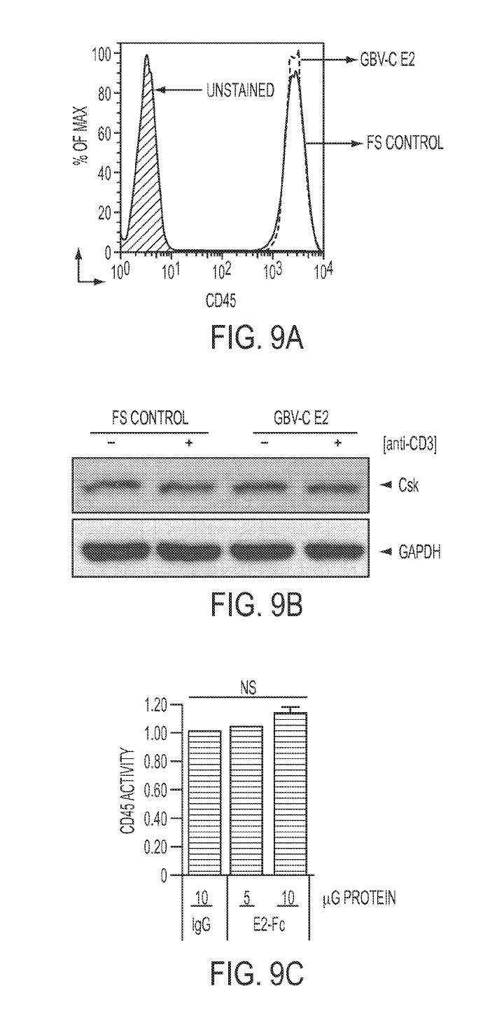

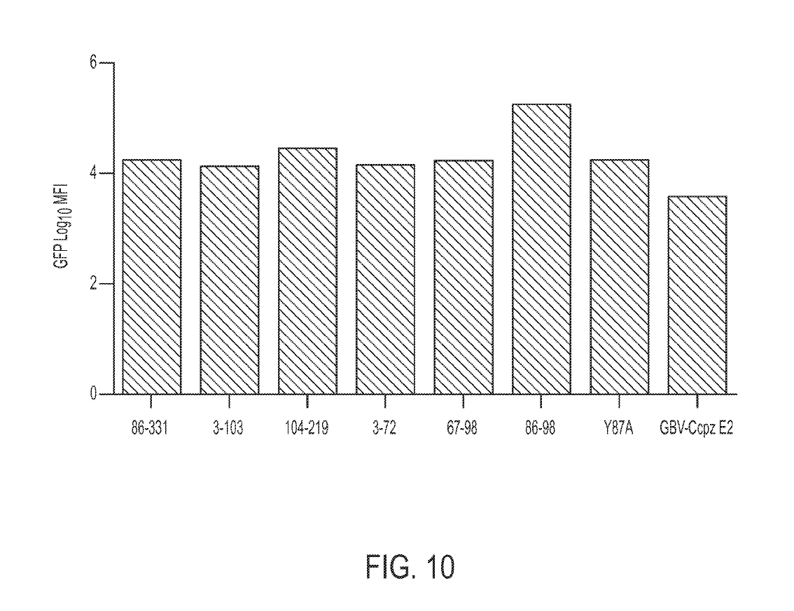

Since GBV-C E2 protein expression inhibited activation following TCR stimulation, the effects of E2 protein on proximal TCR signaling pathways were assessed. Following TCR stimulation, phosphorylation of the linker for activation of T cells (LAT) (FIG. 2C, FIG. 9A) and zeta-chain-associated protein kinase (ZAP)-70 (FIG. 2D, FIG. 9B) was reduced in GBV-C E2 expressing cells compared to the FS control. The reduction in phosphorylation was not due to differences in total cellular LAT or ZAP-70 levels in the E2-expressing and FS control Jurkat cells (FIGS. 2C-D). Thus, expression of GBV-C E2 protein and not the E2 coding RNA inhibited T cell activation by reducing the activation of proximal TCR signaling pathways.

GBV-C E2 Protein Inhibits Lck Activation.

Lymphocyte specific protein tyrosine kinase (Lck) activation is required for signaling through the TCR (Davis and van der Merwe, 2011). Inactive Lck is phosphorylated at tyrosine 505 (Y505) by the C-src tyrosine kinase (Csk). Following TCR engagement, phosphorylated Y505 is dephosphorylated by CD45 tyrosine phosphatase, leading to a change in conformation and subsequent autophosphorylation of Lck tyrosine 394 (Y394) in trans (Davis and van der Merwe, 2011). Lck must be phosphorylated at Y394 to be active, leading to ZAP70 phosphorylation and downstream signaling through the TCR pathway.

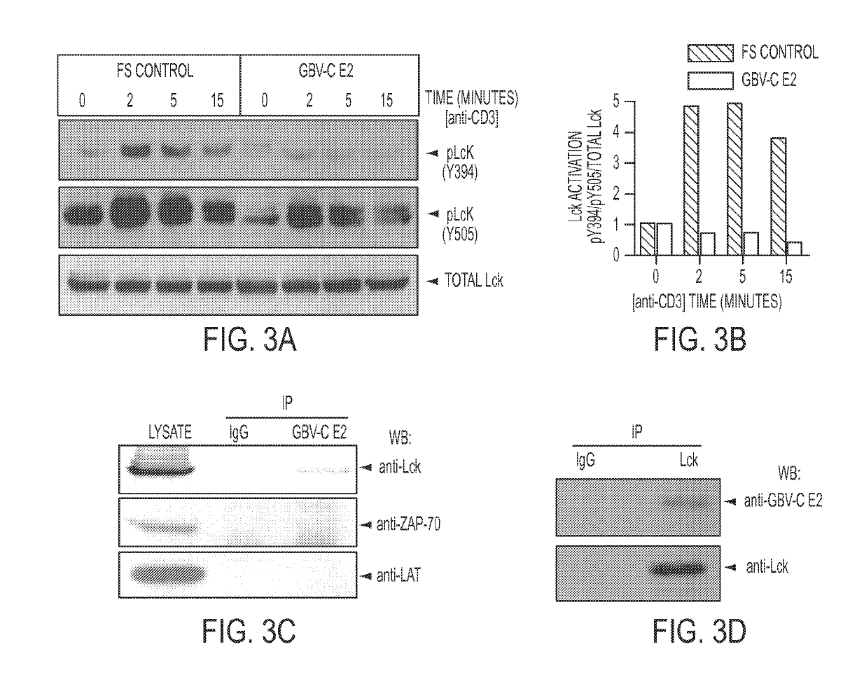

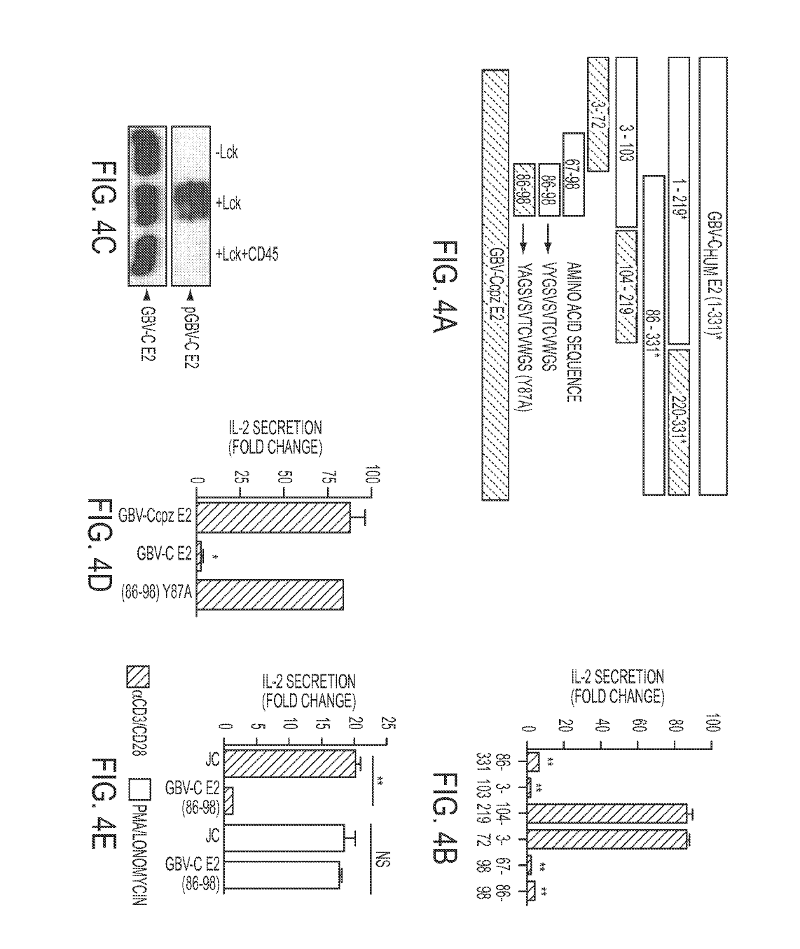

Following TCR engagement with anti-CD3, Lck activation was reduced in Jurkat cells expressing E2 protein compared to FS controls as measured by Y394 phosphorylation (FIGS. 3A-B). This inhibition was not due to altered Lck regulation, as CD45 and Csk expression levels were similar in both GBV-C E2 expressing cells and the FS control cells (FIGS. 9A-B). Furthermore, CD45 phosphatase activity was not altered in vitro by incubation with recombinant GBV-C E2 protein (FIG. 9C).

To determine if GBV-C E2 interacted directly with Lck, recombinant GBV-C E2 protein was incubated with Jurkat cell lysates. The E2-cell lysates were incubated with Lck, ZAP-70 or LAT antibodies to precipitate these proteins. GBV-C E2 protein specifically co-precipitated Lck but not ZAP-70 or LAT (FIG. 3C). Consistent with this finding, pull down of GBV-C E2 from E2 expressing Jurkat cells also specifically precipitated Lck (FIG. 3D), thus the GBV-C E2 protein interacts with Lck, and inhibits Lck activation following TCR stimulation.

A 13 Amino Acid Peptide Domain within GBV-C E2 Inhibits T Cell Receptor (TCR) Signaling.

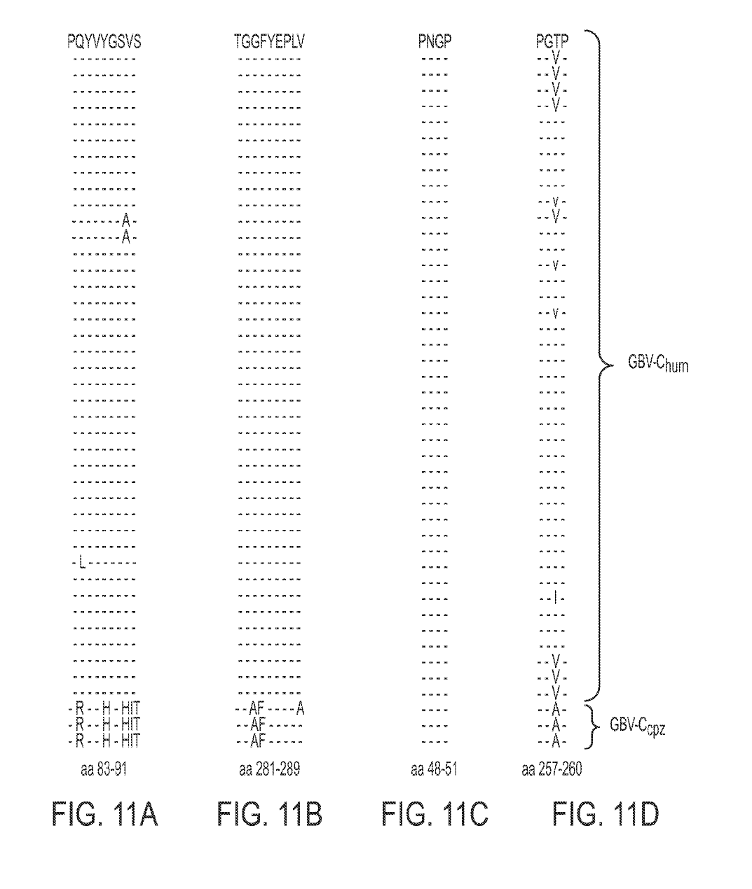

Expression of the N-terminal region 219 aa of GBV-C E2 protein was sufficient to inhibit IL-2 production following TCR stimulation (Bhattarai et al., 2012a). To characterize the region(s) within the GBV-C E2 protein required for TCR signaling inhibition, Jurkat cells expressing truncated GBV-C E2 proteins were generated (FIG. 4A). All cell lines stably expressed GFP detected by flow cytometry (FIG. 11). Following TCR stimulation with anti-CD3/CD28, IL-2 production was blocked in all of the cell lines that contained a 13 amino acid motif within GBV-C E2 (aa 86-98), but not in cell lines that expressed other regions of the E2 protein (FIG. 4B).

Based on kinase-specific phosphorylation substrate prediction programs, the tyrosine residue at position 87 (Y87) in GBV-C E2 is predicted to be an Lck (Src-kinase) target (FIG. 4A; Obenauer et al., 2003; Xue et al., 2008). Although the kinase substrate prediction required the sequence of E2 to begin at position 83, the 13-mer peptide sequence (86-98 aa) contains upstream vector amino acid sequences which maintain the site as a predicted Lck substrate. Consistent with this, recombinant Lck phosphorylated recombinant E2 in an in vitro kinase assay (FIG. 4C). Similar to Lck, the GBV-C E2 protein was dephosphorylated by CD45 tyrosine phosphatase (FIG. 4C).

The predicted Lck substrate motif within GBV-C E2 (aa 83-91; PQYVYGSVS) is highly conserved among GBV-C isolates. There is complete homology among 39 of 42 published human GBV-C isolates representing all seven GBV-C genotypes (FIG. 11A). The three isolates that differed had only a single aa difference (Q84L or V90A), and these polymorphisms maintained a predicted Lck phosphorylation site. In contrast, this E2 protein sequence in chimpanzee GBV-C variant isolates (GBV-Ccpz) differed significantly (PRYVHGHIT; FIG. 11A). The GBV-Ccpz E2 protein has a histidine residue at position 87 (H87) instead of a tyrosine, and this sequence is not predicted to be phosphorylated by Lck.

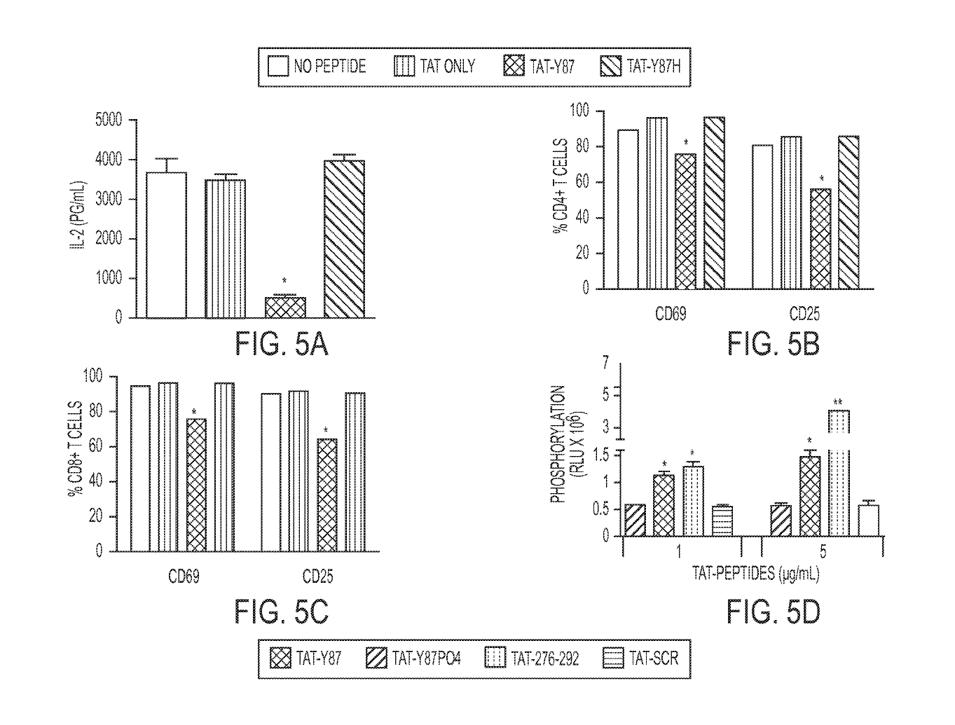

Consistent with this, expression of the GBV-Ccpz E2 protein in Jurkat cells did not inhibit IL-2 production following TCR stimulation (FIG. 5A), and expression of the human GBV-C E2 peptide motif with a tyrosine to alanine substitution at aa 87 (Y87A) did not inhibit TCR signaling (FIG. 5A). There is a second predicted Lck phosphorylation substrate motif that is also highly conserved within the GBV-C E2 protein (aa 281-289, TGGFYEPLV; FIG. 11B). Previous studies and additional mapping expressing E2 proteins in Jurkat cells (FIG. 4A) demonstrated that this region of E2 does not inhibit TCR-mediated activation (Bhattarai et al., 2012a) (FIG. 4A). GBV-C E2 protein also contains two well conserved Src homology domain 3 (SH3) binding domains (PXXP; aa 48-51 and 257-260; FIGS. 11C-D) (Alexandropoulos et al., 1995; Saksela et al., 1995). Neither of these regions was required for inhibition of TCR signaling (FIGS. 4A-B) (Bhattarai et al., 2012a).

To determine if the effect of GBV-C E2 protein on activation was specific for TCR-mediated signaling, control Jurkat cells or Jurkat cells expressing the human GBVC E2 (86-98 aa) were stimulated with phorbol-12-myristate-13-acetate (PMA) and ionomycin, which bypass the TCR for activation of T cells. Incubation of Jurkat cells in PMA-ionomycin did not activate Lck (FIG. 4B), and the GBV-C E2 peptide (86-98) did not inhibit IL-2 release in PMA-ionomycin-stimulated cells (FIG. 4C). Thus, a highly conserved region within the GBV-C E2 protein contains a predicted Lck substrate motif, and expression of this region in CD4+ T cells inhibited TCR-mediated signaling. GBV-C E2 protein did not inhibit activation of T cells through non-TCR pathways (PMA-ionomycin).

Synthetic GBV-C E2 Peptides Inhibit TCR Activation in Primary Human T Cells.

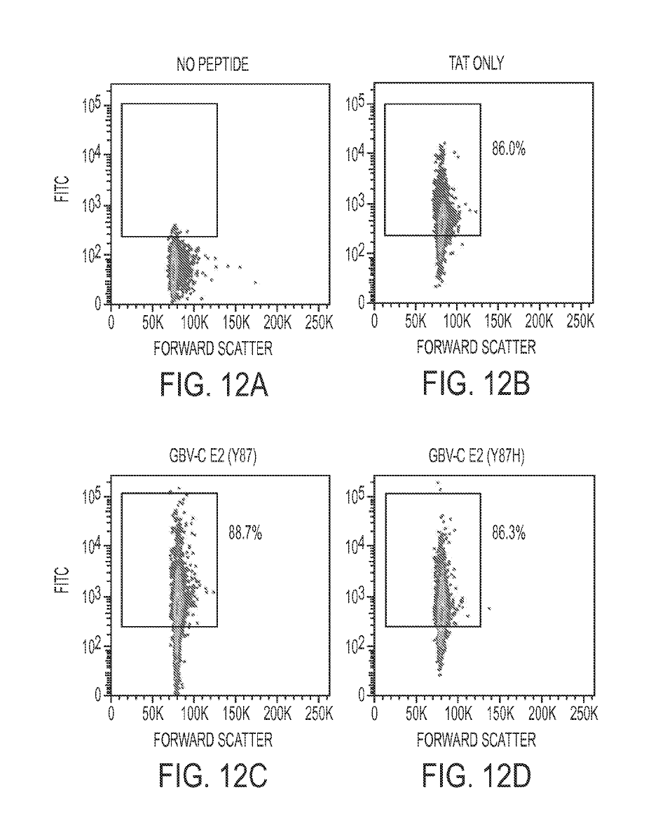

To confirm that the predicted Lck substrate motif within GBV-C E2 protein was sufficient to inhibit TCR-mediated signaling in primary human CD4+ and CD8+ T cells, synthetic peptides with the native sequence (aa 86-101), or with a histidine substituted for the tyrosine at aa 87 (Y87H) were generated and tested for their abilities to inhibit TCR-mediated activation. The peptides were biotinylated to monitor cellular uptake, and included an N-terminal HIV Tat protein transduction domain (TAT) to promote internalization by target cells. A TAT only synthetic peptide served as a negative control. All three biotinylated peptides were internalized by healthy human PBMCs as demonstrated by flow cytometry (FIGS. 12A-D). Following TCR stimulation, IL-2 production by PBMCs was inhibited in cells incubated with the TATY87 peptide, but not in those incubated with either the TAT-Y87H or the TAT control peptides (FIG. 5A). Similarly, surface expression of T cell activation markers CD69 and CD25 was significantly reduced in primary human CD4+ and CD8+ T cells incubated with the TAT-Y87 peptide compared to mutant or control peptide (FIGS. 5B-C). The TAT-Y87 peptide was phosphorylated by Lck in a dose-dependent manner in an in vitro kinase assay (FIG. 5D); however, a synthetically phosphorylated Y87 peptide (TAT-Y87PO4) did not serve as an Lck substrate (FIG. 5D). Thus, this peptide serves as an Lck substrate and will compete for phosphorylation with Lck. As noted, there is a second predicted Lck substrate motif within GBV-C E2 (aa 281-289) that did not inhibit TCR-mediated IL-2 production (Bhattarai et al., 2012a). However, the synthetic peptide containing this motif (TAT-276-292) served as an in vitro Lck substrate (FIG. 5D) while a control peptide with the same E2 amino acids (aa 281-289) synthesized in a scrambled order (TAT-SCR) was not phosphorylated by Lck in vitro (FIG. 5D). Together, these data demonstrate that synthetic peptides representing Lck substrate motifs within GBV-C E2 protein are phosphorylated by Lck and inhibit TCR signaling in human T cells.

GBV-C E2 Protein Inhibits T Cell Activation in Bystander Cells.

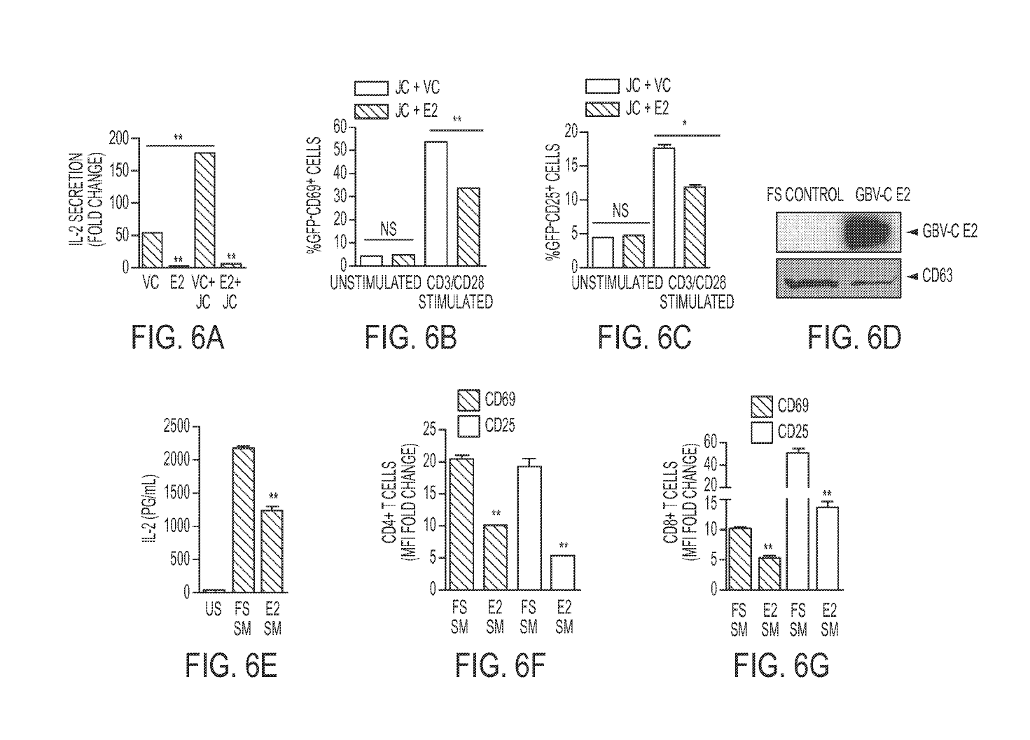

Since GBV-C E2 protein expression inhibited TCR signaling (FIGS. 2A-C), the inventors hypothesized that E2 expressing cells may inhibit TCR signaling in bystander cells contributing to global reduction in TCR signaling that has been observed in GBV-C infected subjects (Bhattarai et al., 2012b; Maidana-Giret et al., 2009; Stapleton et al., 2012; Stapleton et al., 2009). To test this hypothesis, GBV-C E2 expressing (GFP positive) or vector control Jurkat cells (VC; also GFP positive) were co-cultured with Jurkat cells not expressing GFP. Following TCR engagement, IL-2 secretion FIG. 6A) and surface expression of the activation markers CD69 and CD25 (FIGS. 6B and 6C) were significantly inhibited in the bystander Jurkat cells co-cultured with GBV-C E2 expressing cells compared to bystander cells co-cultured with the vector control cells. Since serum extracellular microvesicles (EMV) from GBV-C infected subjects inhibited TCR signaling when incubated with primary human T cells (FIGS. 1A-F), the inventors examined Jurkat cell supernatants for EMV. GBV-C E2 protein was detected in EMV purified from E2-expressing Jurkat cell culture supernatant but not in EMV from the FS supernatant fluid (FIG. 6D). Both E2-expressing and the FS expressing Jurkat cells released EMV that contained CD63 (FIG. 6D), supporting an endocytic origin (Meckes and Raab-Traub, 2011), and consistent with the findings of EMV present in GBV-C infected human serum (FIGS. 1B-C).

To determine if GBV-C E2 protein released from Jurkat cells reduced TCR signaling in bystander T cells, primary human CD4+ and CD8+ T cells from healthy blood donors were incubated with EMV purified from E2-expressing Jurkat cells (E2 EMV) or FS control Jurkat cells (FS EMV). Following TCR engagement, IL-2 release (FIG. 6E) and cell surface expression of CD69 and CD25 (FIGS. 6F-G) were significantly reduced in cells incubated with E2 EMV compared to cells incubated with FS EMV. Thus the GBV-C E2 protein expressing cells inhibited TCR signaling in bystander T cells, and GBV-C E2 protein released from Jurkat cells was contained within EMV. The GBV-C E2-containing EMV inhibited TCR signaling in bystander T cells.

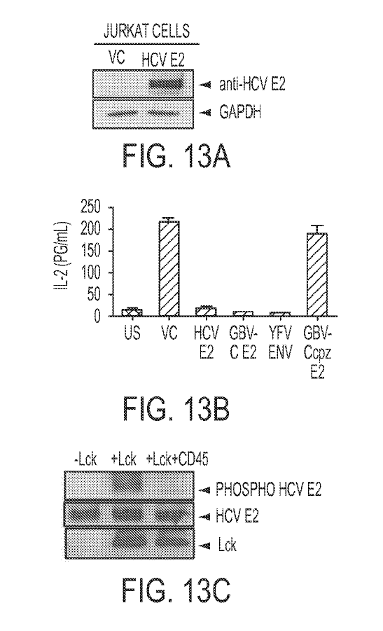

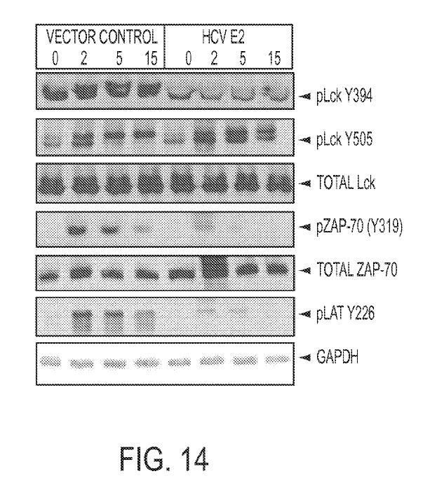

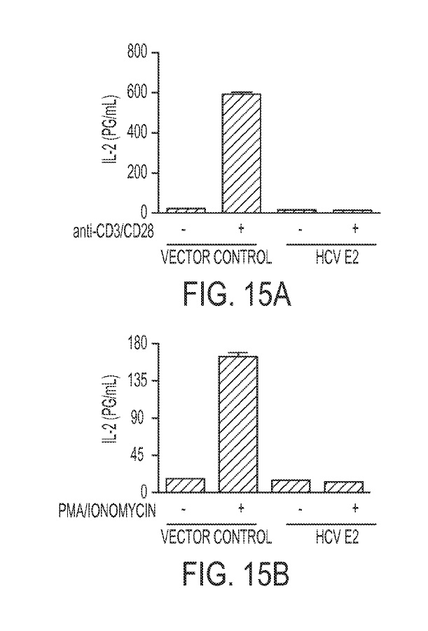

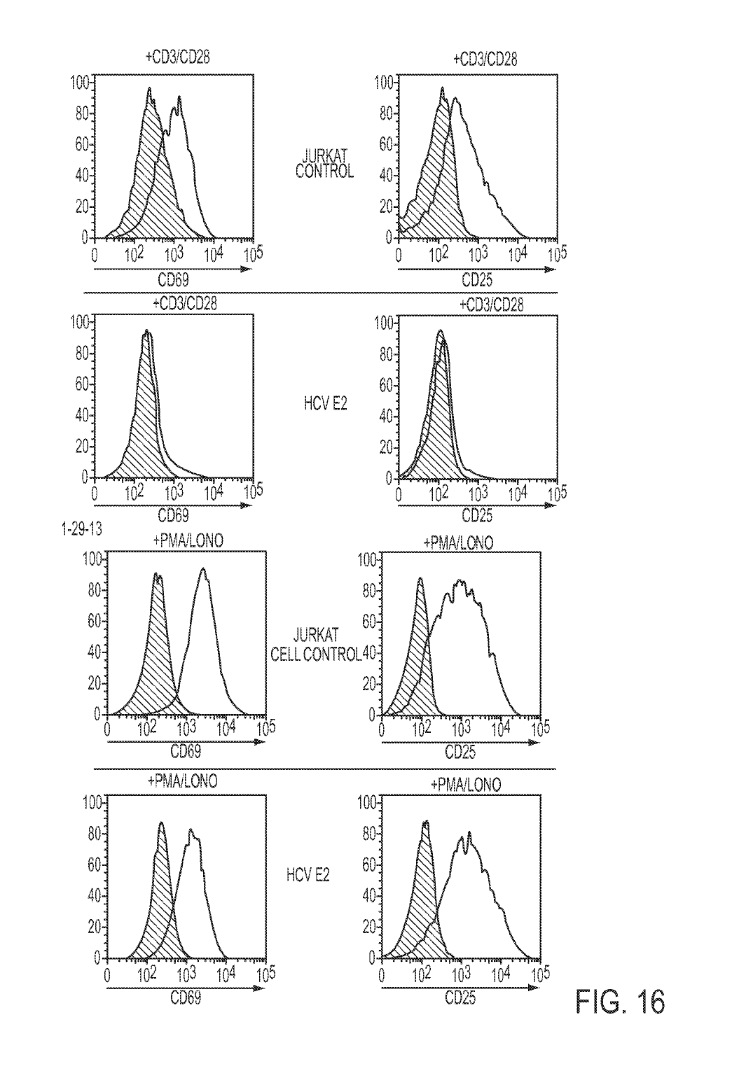

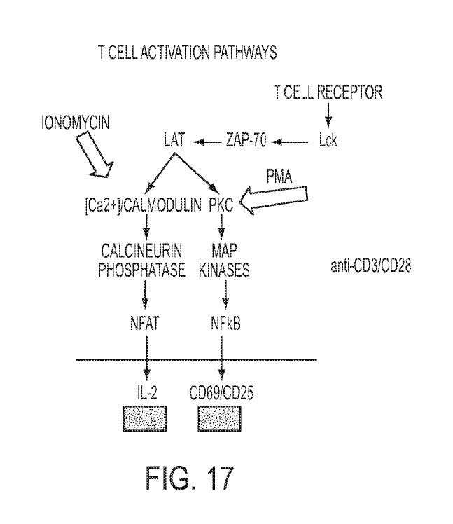

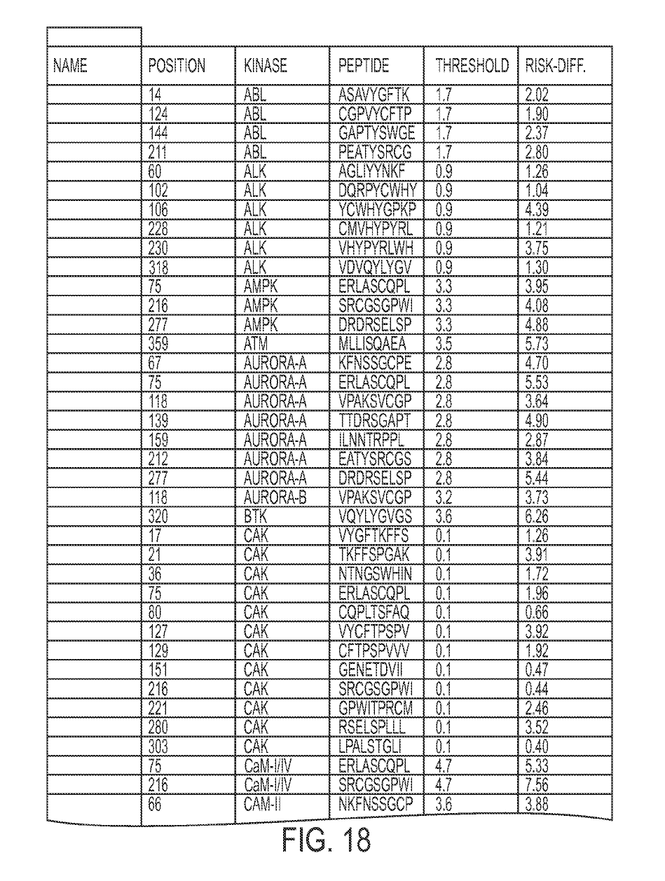

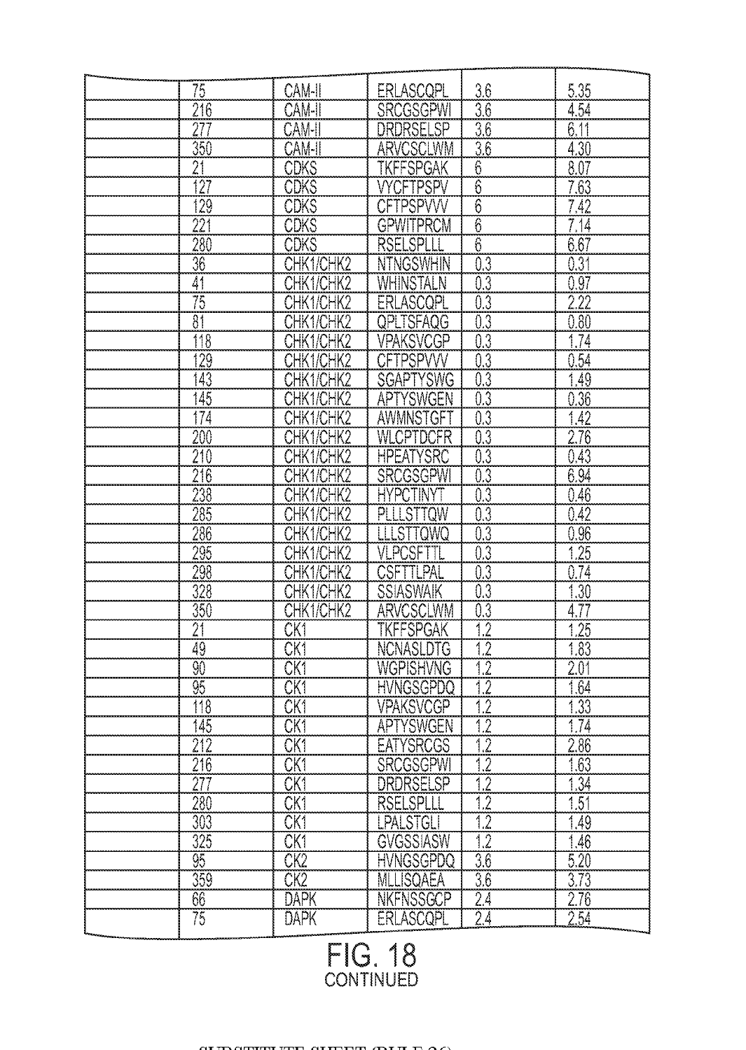

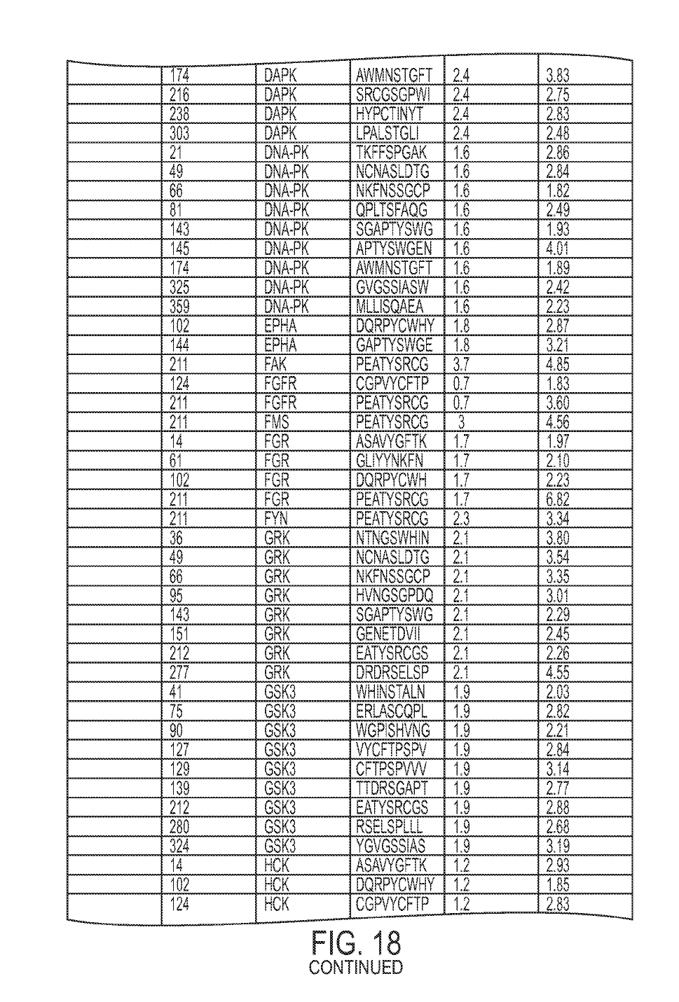

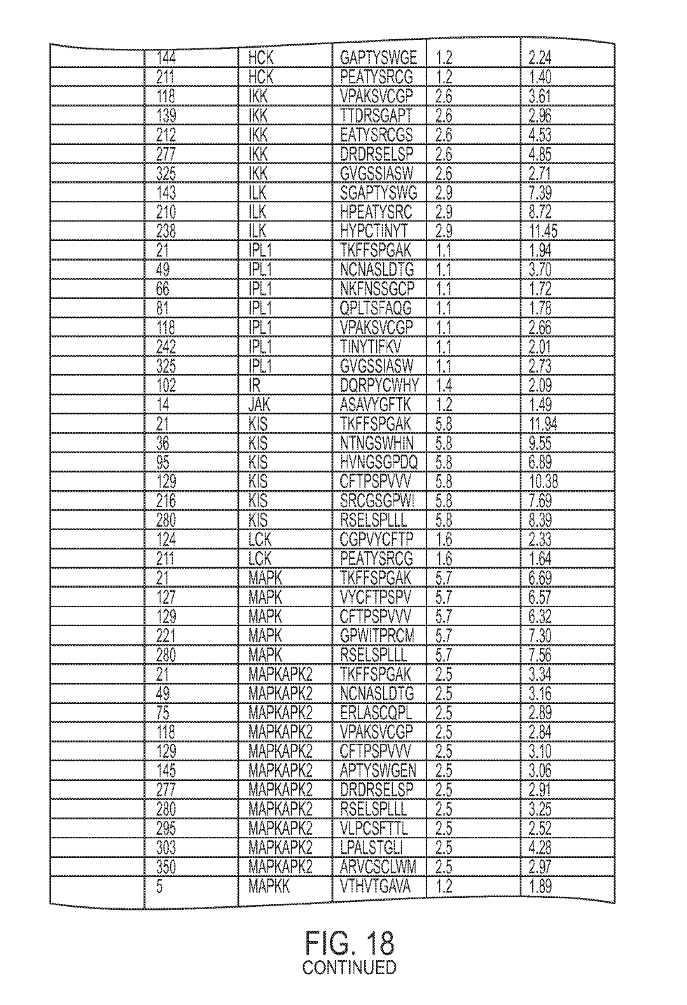

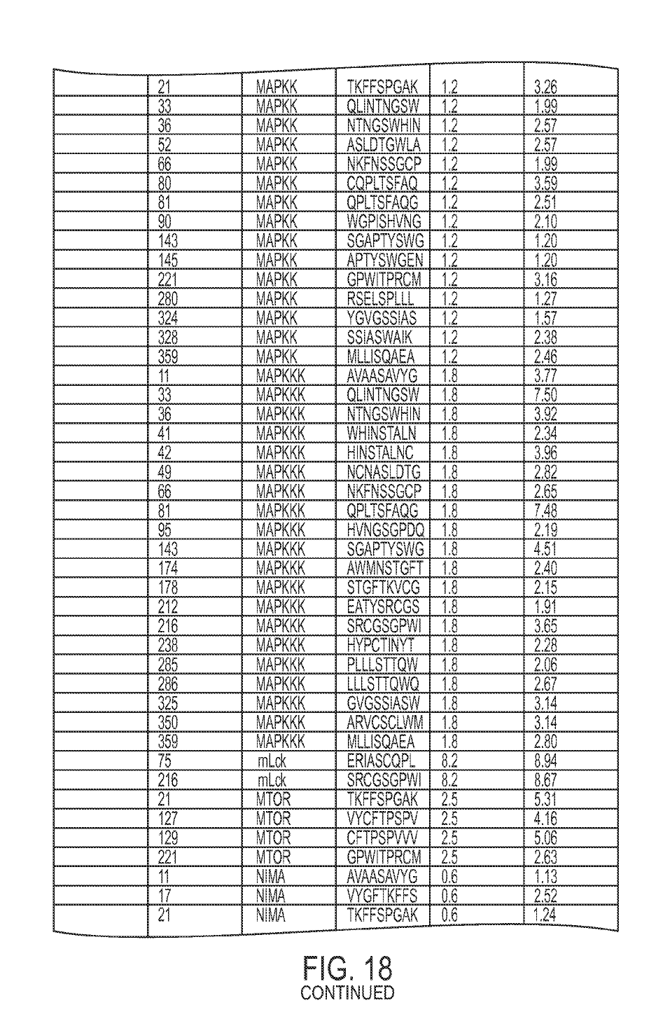

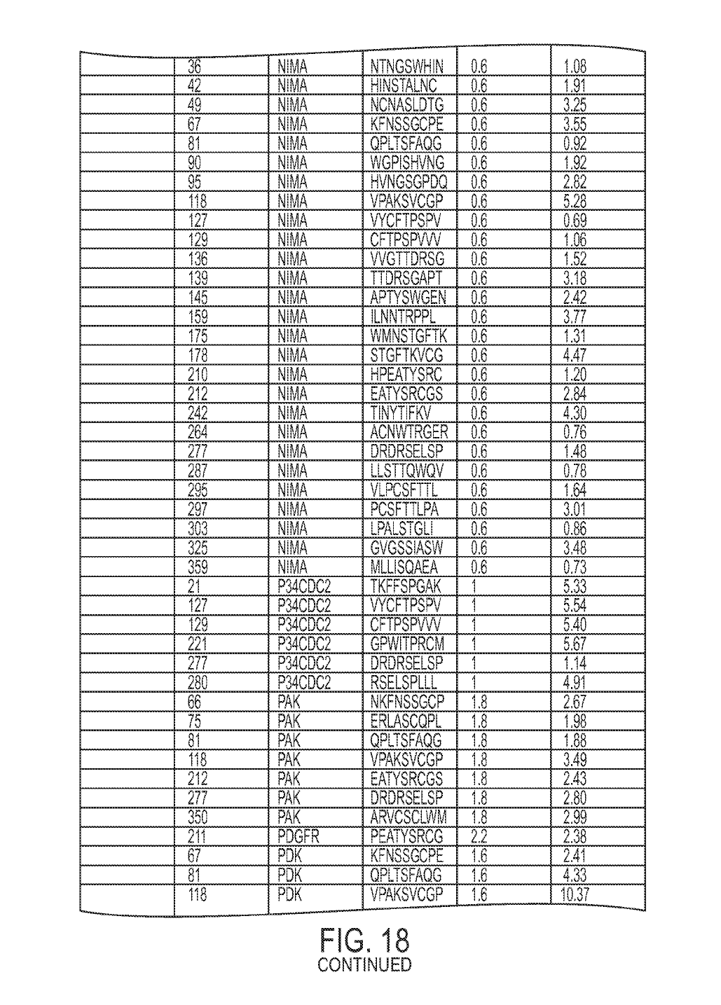

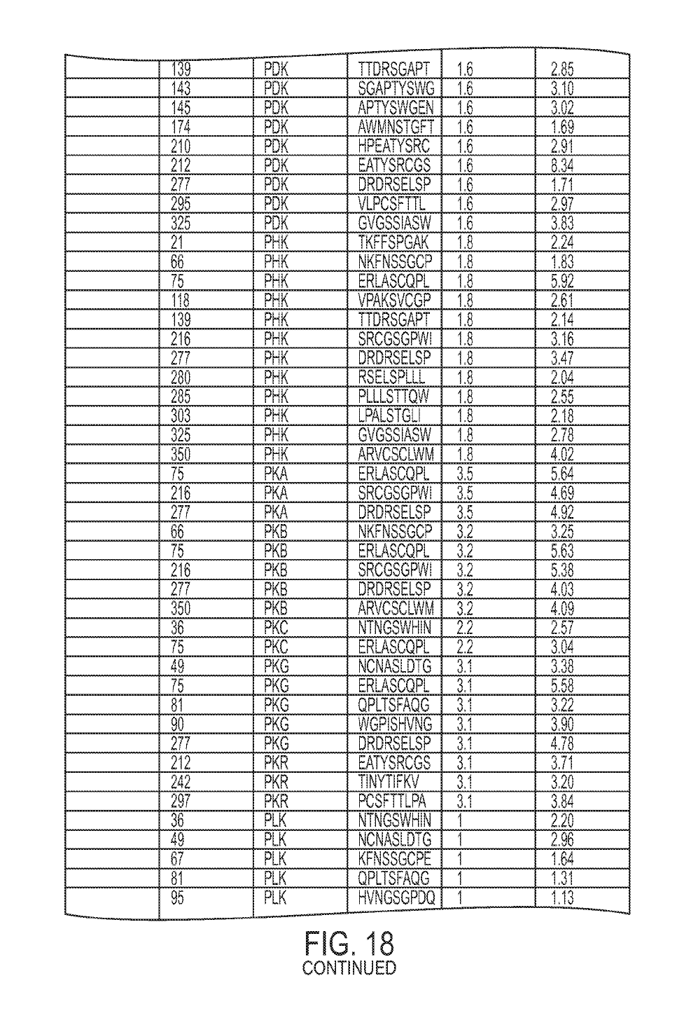

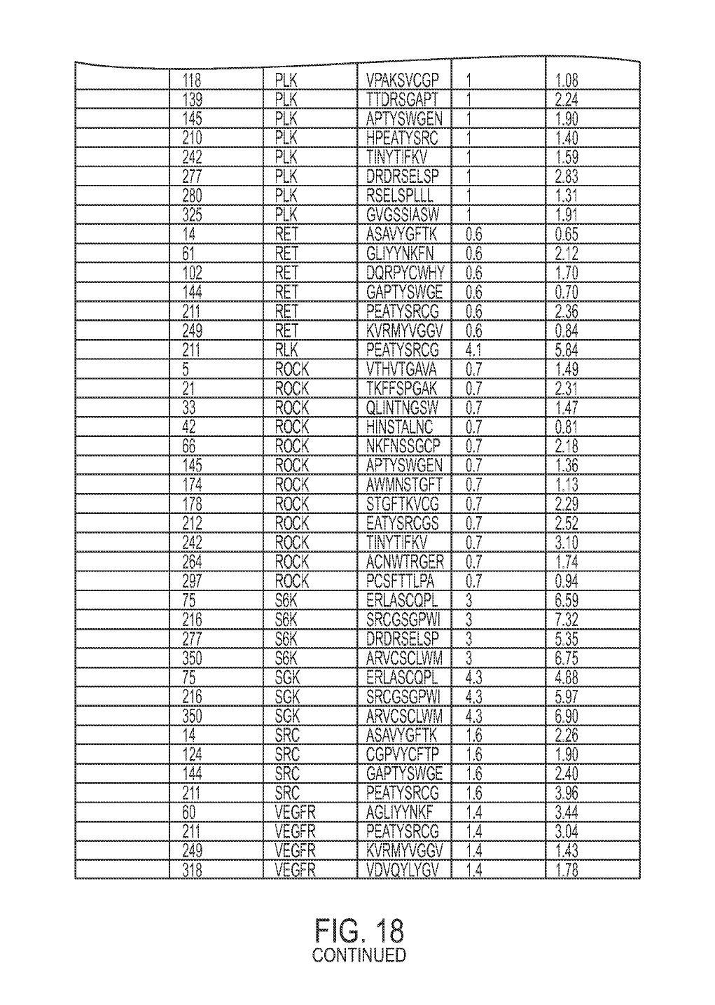

Expression of the HCV E2 protein (expression in Jurkat cells shown in FIG. 13A), and the YFV envelope protein potently inhibit IL-2 release following TCR activation (FIG. 13B). Recombinant HCV E2 was also phosphorylated in vitro by recombinant Lck (FIG. 13C), and upregulation of the activation markers (CD69 and CD25), similar to that observed for GBV-C (FIGS. 13A-C). HCV E2 protein blocked activation of Lck following anti-CD3 antibody as measured by phosphorylation increase in Y394 (FIG. 14). Consistent with this, downstream TCR-signaling molecules ZAP70 and LAT had reduced activation, similarly measured by assessing phosphorylation of Y319 (ZAP70) and Y226 of LAT (FIG. 14). The inventors previously found that the block in T cell signaling mediated by the GBV-C E2 protein is specific for the TCR, as PMA and ionomycin bypass the TCR, and E2 does not block PMA/ionomycin-mediated activation). Like GBV-C E2 protein expression, Jurkat cells expressing HCV E2 demonstrated a complete block in IL-2 release with anti-CD3/CD28 (FIGS. 15A-B). However, and in contrast to GBV-C E2 protein, HCV E2 protein expression in Jurkat cells also blocked the release of IL-2 release following activation with PMA-ionomycin (FIG. 16). Since PMA-ionomycin bypass the TCR, this indicates downstream inhibition of T cell activation events in addition to any effects on Lck (FIG. 16). PMA and ionomycin stimulate different T cell activation pathways (FIG. 17). Since PMA mediates CD69 and CD25 regulation, this pathway is not inhibited by HCV E2. However, ionomycin regulates IL-2 release via NFAT effects on IL-2 transcription. Thus, HCV E2 blocks both the Lck signaling and signaling molecules within the ionomycin-inhibit-able pathway (FIG. 16). Further characterization of the mechanisms by which this occurs are underway.

Since HCV replicates in hepatocytes, the mechanism of T cell modulation described in several clinical observations (Bhattarai et al., 2012c; Fournillier et al., 2001; Cerny and Chisari, 1999) is not obvious from this work. However, two lines of evidence indicate that the proposed mechanism of global T cell inhibition described in prior work (unpublished) are operative in HCV infection. First, evidence was presented in 2004 that serum from patients with HCV infection released exosomes containing HCV particles, and that cell culture lines expressing HCV E2 released exosomes containing E2 protein in vitro (Masciopinto et al., 2004). Secondly, recent data indicate that HCV-infected hepatocyte cell lines release exosomes which interact with dendritic cells leading to the release of antiviral cytokines which influence local inflammation (Cerny and Chisari, 1999). As the inventors have demonstrated with GBV-C serum-derived microvesicles, preliminary data demonstrate that serum microvesicles precipitated by an exosome-enrichment product (exoquick) inhibit TCR-signaling in primary human T cells (not shown). Thus, there are limited and not well accepted data suggesting that HCV replicates in lymphocytes, the release of exosomes and other microvesicular bodies from infected hepatocytes will interact with T cells in lymphoid tissue and peripheral blood, providing the mechanism by which HCV E2 interferes with T cell activation.

Bioinformatic analysis identifies several amino acid sequences in HCV and GBV-C E2 proteins that may serve as a substrate for several important signaling molecules in T and B cells, and as noted for GBV-C, these prediction algorithms require experimental confirmation. The HCV E2 protein we amplified from an Iowa patient contains 5 potential Lck substrate targets. One of these is highly conserved among different genotypes of HCV. Interestingly, the number of Lck sites on different HCV genotypes varies considerably, and if more than one are functional, this may explain differences in HCV pathogenicity between different genotypes. Nevertheless, because the HCV E2 sequence expressed in Jurkat cells potently blocks TCR signaling (FIGS. 14-16), the inventors are mapping the sites on HCV E2 protein for inhibition of Lck and the ionomycin-inducible pathways by deletion mutagenesis. These will be allow selection of key amino acid substitutions that will abrogate the T cell activation inhibition while retaining antigenicity and enhancing immunogenicity. Although HCV is highly variable, conserved epitopes that are broadly neutralizing have been identified (Potter et al., 2012) and improved antigen presentation and memory will enhance vaccine-induced immune responses.

YFV.

The YFV envelope is predicted to have conserved Lck substrate sites. HCV & HPgV do not have robust in vitro lymphocyte replication, where as prior reports suggest YFV will replicate in T cells. Thus, the inventor's hypothesis was that Flavivirus envelope proteins share a conserved interaction with Lck, innately interfering with T cell activation and proliferation. YFV envelope and replication interactions with T cells were assessed.

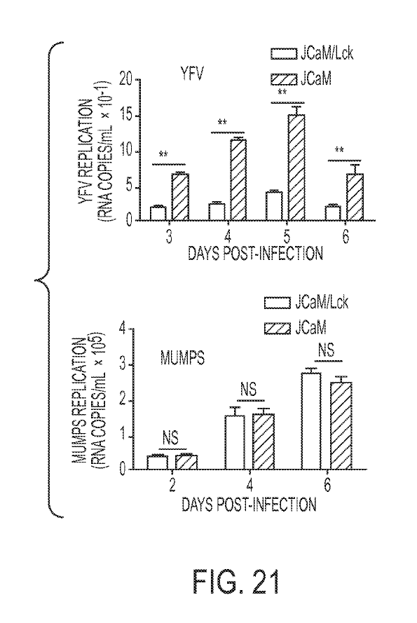

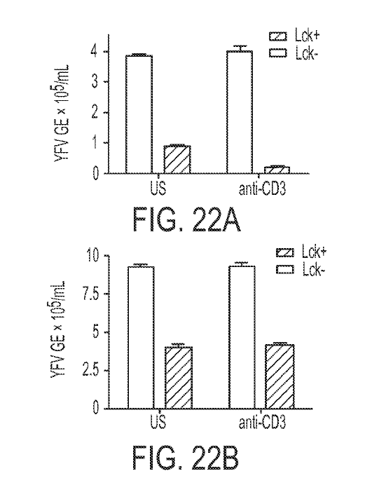

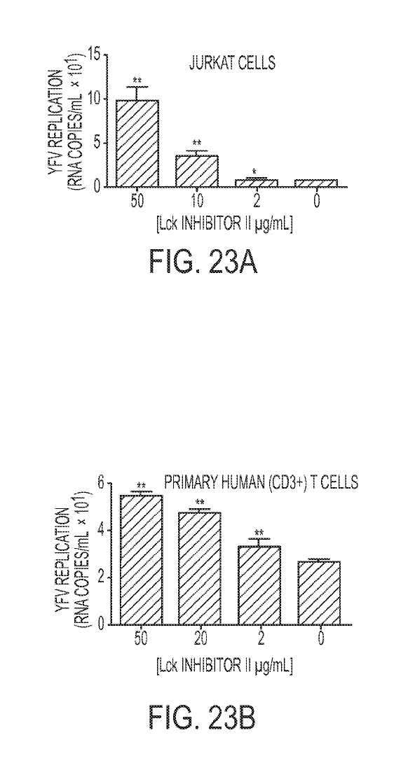

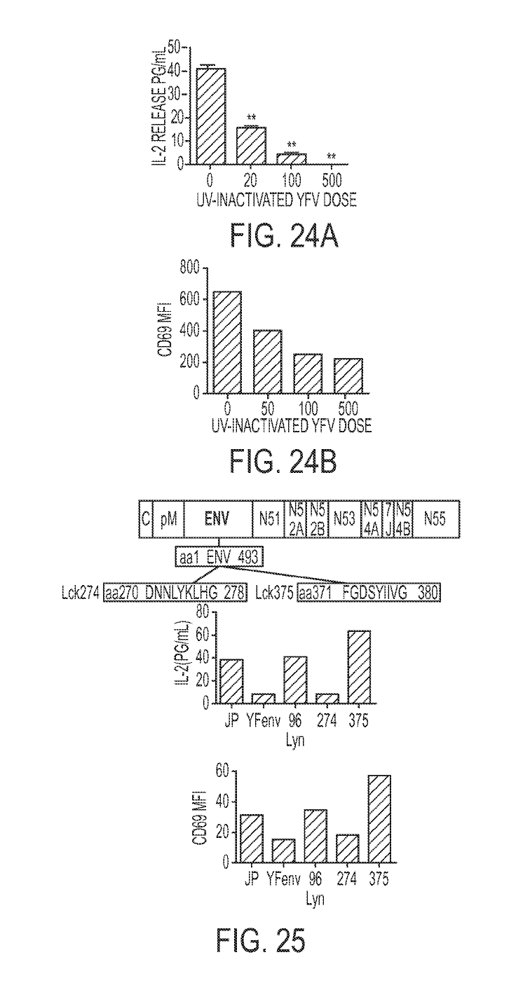

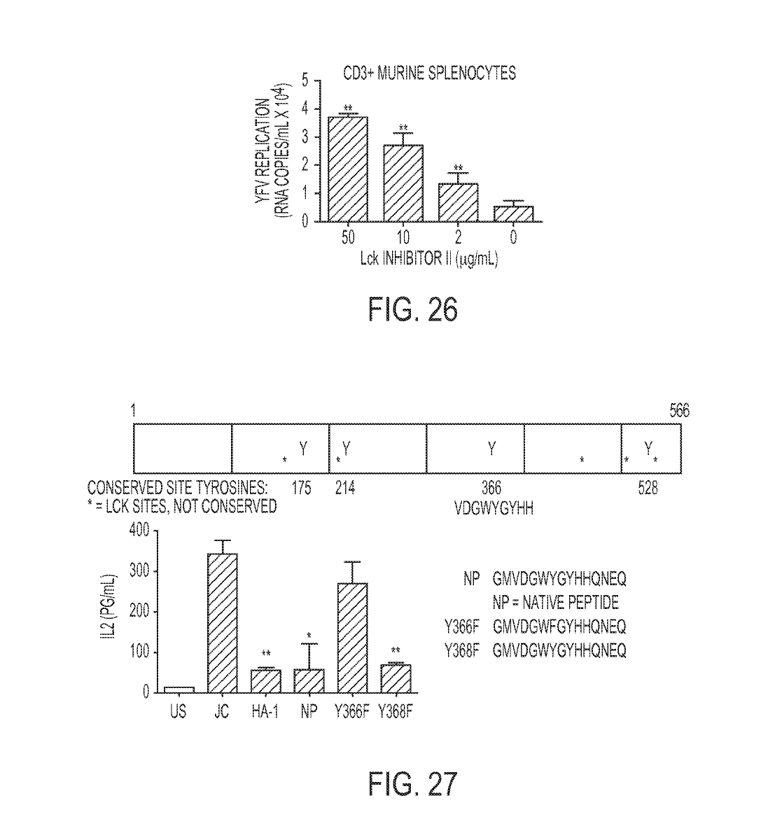

T cell activation with anti-CD3/CD28 or PMA-Ionomycin prior to YFV infection significantly reduced YFV replication in human and murine T cells and a human T cell line. Replication was enhanced in Lck− cells compared to Lck+ cells, and addition of the Lck kinase inhibitor II increased YFV replication in a dose-dependent manner (FIG. 21). T cell infection prior to activation blocked TCR-, and to a lesser extent, P-I-mediated activation (FIGS. 22A-B; 23A-B). UV-inactivated YFV also inhibited TCR-mediated activation of primary T cells (FIGS. 24A-B). Expression of YFV env and two peptides containing predicted Lck substrate sites inhibited TCR-activation in Jurkat cells, but not P-I activation (FIG. 25).

Conserved Lck substrate sites are present on GBV-C, HCV, and YFV envelope proteins. These may represent a mechanism to evade host immune responses, and interfere with immune potency by innately interfering with T cell function. Studies are underway to determine how these proteins are able to interact with Lck in the context of virus particles (FIGS. 24A-B) or when added to cells as synthetic peptides (data not shown).

In conclusion, YFV interacts with T cells, blunting TCR-activation. This does not require replication. Expression of YFV env blocks activation at the level of Lck, suggesting that env interacts with and competes for Lck phosphorylation. These data suggest that a conserved TCR-inhibition mechanism exists among env proteins of the Flaviviridae. Further studies to gain insight into this virus particle-mediated immune suppressive effect are underway.

Influenza.

To determine if T cell inhibitory motifs occur in other viruses, the inventors examined human, avian, and swine influenza HA sequences for evidence of conserved tyrosines predicted to be substrates for Lck. HA amino acid sequences (2,978 unique) from human (H1, H2, H3), avian (H1-H13, including H5 and H7) and swine (H1, H3) were aligned. Conserved tyrosines were identified using the NCBI Influenza Virus Resource Information site (Bao et al., 2007). Consensus sequences were examined using a Bayesian decision theory-based online program that predicts PK-specific phosphorylation sites (PPSP) (Xue et al., 2008; Xu et al., 2014), and conserved tyrosine sites predicted to be Lck substrates are shown in Table 5.

TABLE 5
Bioinformatic analyses of predicted and conserved influenza Lck substrate
sites

| Years | Type | Sequences/isolates | Conserved Lck | Location on consensus sequence |
|---|---|---|---|---|
| 1910-1997 | A, H1-H3 | 305/516 | 6 | 122, 182, 216, 373, 513 |
| 1998-2002 | A, H1-H3 | 190/327 | 4 | 125, 213, 377, 517 |
| 2006-2007 | A, H1-H3 | 350/692 | 4 | 125, 186, 377, 512 |
| 2011 | A, H1-H3 | 355/559 | 4 | 125, 185, 376, 516 |
| 1910-2011 | B | 432/926 | 1 | 313 |
| human A isolates: | | 1242/3024 | | |
| 1927-2000 | Avian H1- | 570/748 | 5 | 186, 220, 384, 519, 549 |
| | H13 | | | |
| 2010-2012 | Avian H5 | 24/30 | 12 | 112, 171, 176, 205, 211, 268, |
| | | | | 285, 364, 459, 499, 524, 530 |
| 2010-2012 | Avian H7 | 50/113 | 5 | 133, 153, 359, 456, 499 |
| 1942-2010 | Swine H1-H3 | 683/958 | 6 | 175, 190, 222, 382, 476, 516 |

| | |
|---|---|
| Total isolates examined: | 2969/4659 |

Lck sites predicted using the consensus sequence for each date interval noted (HA types indicated). Sites numbered using consensus sequences (thus numbering will vary-e.g., the Y366 in pH1N1 [FIG. 27] = Y376 in table). Additional tyrosines were predicted to be Lck sites; however, these sites were not as highly conserved. For type B, an additional Y was a predicted Lck substrate, but was incompletely conserved.

To determine if influenza A virus (IAV) HA interferes with TCR-mediated activation, a representative pH1N1 HA coding sequence was expressed in a human T cell line (Tet-Off) as described for HCV and GBV-C E2 (Bhattarai et al.; 2012; 2013; Xiang et al., 2006; McLinden et al., 2006; Xiang et al., 2008; Dhanasekaran et al., 2012) (FIG. 27). T cell lines expressing full-length pH1N1 HA protein or peptide regions were generated. Cells expressing the full-length HA inhibited IL-2 release compared to parental Jurkat cells (JC) following stimulation with anti-CD3/CD28 (FIG. 27; *=p<0.05, **=p<0.01 compared to JC). All predicted Lck substrate sites for the strain used are shown (* or Y). Cells expressing a peptide motif containing the most conserved Lck site (Y366) or mutants (Y366F and Y368F) were generated. Similar to the full-length HA, the native 360-374 peptide and the Y368F peptide inhibited TCR-mediated IL-2 release compared to the control. However, mutation of the predicted Lck substrate (Y366F) abolished the inhibition of TCR signaling (FIG. 27). Y368 is not predicted to be a target for Lck phosphorylation, and demonstrating the specificity of Y366 for the TCR inhibition. US=unstimulated. HA-1 is one of two independent cell lines generated that expressed pH1N1 HA. The second cell line (HA-2) also inhibited TCR-mediated signaling (data not shown). Although additional Lck sites are present on the HA that could regulate TCR signaling, some of which are conserved among all isolates, expressing just the Lck site with Y366 is sufficient to inhibit TCR. This work was funded by internal funds and due to limited resources, funding to pursue this work further are not available.

In summary, three flavivirus envelope proteins (GBV-C, HCV, YFV) and a pH1N1 HA protein contain TCR-inhibitory motifs that reduce T cell activation and proliferation. Since envelope proteins are the first viral proteins seen by immune cells, and T effector functions require TCR-mediated cell activation, interference with T cell activation will reduce T cell responses to foreign antigens. This effect likely contributes to the development of virus persistence for HCV and GBV-C, and to a slower or less potent antibody response due to impaired antigen presentation for influenza. The inhibition is not complete, otherwise GBV-C-, HCV-, and influenza-infected people would have life-threatening immunosuppression. However, clinical data are consistent with reported GBV-C associated reduced immune activation (Stapleton et al., 2009; Nattermann et al., 2003; Maidana et al., 2009; Stapleton et al., 2012; Stapleton et al., 2013; Schwarze-Zander et al., 2010; Bhattarai et al., 2012), mild clinical immune suppression in HCV-infected individuals (Pereira et al., 1998; Lechner et al., 2000; Hahn, 2003; Kittleson et al., 2000; Soguero et al., 2002; Isaguliants et al., 2004), and a poorly immunogenic influenza HA protein (Brydak and Machala, 200; Ghendon, 1990; Seidman et al., 2012).

### Example 3

### Discussion

GBV-C and the related HCV are the only two cytoplasmic RNA viruses that commonly cause persistent human infection. Among HIV-infected people, persistent GBV-C co-infection is associated with prolonged survival, reduced T cell activation and altered IL-2 signaling (Bhattarai et al., 2012a; Bhattarai et al., 2012b; Maidana-Giret et al., 2009; Rydze et al., 2012; Stapleton et al., 2012; Stapleton et al., 2009). The IL-2 signaling defect is due, at least in part, to inhibition of TCR signaling by the envelope glycoprotein E2 (Bhattarai et al., 2012a) and these T cell activation and IL-2 signaling effects may contribute to viral persistence (Bhattarai and Stapleton, 2012). In addition, antibodies to GBV-C proteins are usually not detected during viremia, suggesting an impairment in B cell function (Stapleton et al., 2011). This may reflect altered antigen presentation.

Although there is an association between GBV-C infection and reduced levels of global T cell activation (Bhattarai et al., 2012b; Maidana-Giret et al., 2009; Stapleton et al., 2012), only a small proportion of T cells contained viral genomes. Thus, virus and viral components present in virions, extracellular microvesicles, or virus-infected cells must interact with and inhibit activation of uninfected bystander T cells. The inventors show that extracellular microvesicles present in sera obtained from GBV-C infected subjects, and released by E2-expressing Jurkat cells inhibited TCR signaling in bystander primary human T cells. This is accomplished by reducing the activation of Lck, the proximal tyrosine kinase phosphorylated in the TCR signaling cascade. The data are consistent with the transfer of GBV-C E2 protein within virus particles or in EMV to bystander cells with resultant TCR-signaling inhibition. Since the average GBV-C RNA concentration in infected humans is greater than 1×10⁷ genome copies/mL of plasma, and the virus is produced by T cells (Rydze et al., 2012), lymphoid tissue is constantly exposed to high concentrations of GBV-C E2 protein in infected humans.

Synthetic peptides containing only one of the two predicted Lck substrate motifs on GBV-C E2 protein inhibited TCR signaling in the CD4+ T cell line and in primary human CD4+ and CD8+ T cells (Y87). Although the tyrosine at aa 285 was phosphorylated by Lck in vitro, this region of E2 did not inhibit TCR-mediated activation. This may reflect a lack of access of this E2 region to Lck, as the Y285 is not predicted to be the surface of the protein based on structural models of the related HCV E2 (Krey et al., 2010). This also suggests that not all predicted tyrosine kinase substrate motifs on viral structural proteins will display functional activity. GBV-C E2 protein bound to Lck in reciprocal co-immunoprecipitation experiments, most likely through interactions between SH3 binding domains on the GBV-C E2 protein and the SH3 domain on Lck. Although the SH3 binding regions were not required for TCR signaling inhibition, it is possible that either of these SH3 binding domains may contribute to Lck inhibition in the setting of natural infection.

The E2 Lck substrate motif (aa 83-91) that inhibited TCR signaling is highly conserved in human GBV-C (GBV-Chum) isolates, but is absent in chimpanzee GBV-C (GBV-Ccpz) isolates. Expression of GBV-Ccpz E2 protein did not inhibit TCR signaling. This observation raises the possibility that T cell activation inhibition is not important for GBV-Ccpz. Since the immune reactivity of chimpanzee lymphocytes is significantly lower than that of human lymphocytes (Soto et al., 2010), it is tempting to speculate that there is less selective pressure for GBV-Ccpz to acquire TCR signaling inhibition mechanisms.

The specificity of E2 protein for TCR-signaling was confirmed, as substitution of alanine or histidine for Y87 abolished the TCR-signaling inhibition, and activation through non-TCR-mediated pathways was not inhibited. Both E2 protein and the peptide served as substrates for Lck-mediated phosphorylation in vitro. GBV-C E2 protein inhibited TCR-mediated activation but not PMA/ionomycin activation, and TCR signaling was reduced, and not completely inhibited by GBV-C E2. Thus, although GBV-C infection reduces global T cell activation, it does not completely block TCR signaling in bystander cells. If it did, this would be disadvantageous, as it would create a state of severe immune suppression and clinical disease (Bhattarai and Stapleton, 2012).

In summary, the GBV-C structural protein E2 inhibits TCR-mediated T cell activation by interacting with Lck and competing for Lck phosphorylation. The inhibition is mediated either by the expression of GBV-C E2 protein within cells, or by the transfer of E2 to bystander cells either in the virion or within serum microvesicular particles. These data identify a novel mechanism by which a viral structural protein interferes with tyrosine kinase function resulting in global inhibition of T cell activation. A recent study using an unbiased approach identified interactions between 70 viral proteins (from 30 different viruses) and 579 host cell proteins from various cell lines. More than half of the host proteins interacting with viral proteins are involved in signal transduction pathways (Pichlmair et al., 2012). Since there are numerous predicted kinase binding and substrate sites encoded in viral structural proteins, it is tempting to speculate that the mechanism by which GBV-C inhibits Lck may apply to other host cell signaling processes, and illustrates the potential for regulation of host cell function in noninfected cells by interactions with virus particles. These interactions may influence viral persistence and viral pathogenesis. Identification of the interactions between viral structural proteins and host cells may facilitate the design of novel and specific antiviral therapies and vaccines.

Subunit vaccines for numerous pathogens have been tested for decades, and with the exception of viral proteins that assemble into virus-like particles, none of these are highly immunogenic. The inventors propose that this is the result of envelope-protein disarming the T cell and B cell response via interference with cellular immune function. Based on bioinformatic analyses of YFV, we expressed the YFV envelope in a CD4+ T cell line, and found that it too inhibits TCR-mediated activation, as measured by IL-2 release (FIG. 13B). There are numerous conserved predicted phosphorylation substrate sites encoded in the YFV envelope protein and in related human and animal viruses in the Flavivirus and Pestivirus genera within the Flaviviridae. Clearly, these data suggest that the T cell inhibition mechanism identified is highly conserved in these viruses. The Pestiviruses cause persistent infection in cattle (BVDV) and pigs (CSFV), and improved vaccines are needed for the agricultural industry (e.g., Ridpath, 2013). Furthermore, safe and effective vaccines against YFV, DENV, WNV, JEV, and other members of the human Flaviviridae are needed.

Several vaccines provide modest to good protection against pathogens, but have poor memory responses resulting in the need for frequent boosting (Schotsaert et al., 2012). Although influenza vaccine generates protective levels of anti-HA antibody within 28 days, by 1 year (usually <3 months), antibody titers are below the limit of detection (Wrammert and Ahmed, 2008). These features, in addition to the issue of antigenic drift, limit the effectiveness of influenza vaccination. Nevertheless, conserved antibodies that cross-protect against numerous influenza strains are possible (Chivero et al., 2012); however, the rapid fall in titer (reflecting memory B cells) and poor immunogenicity remain features that need to be overcome. Using bioinformatics, it is clear that influenza viruses (including A and B) contain T cell modulatory motifs on their hemagluttinin proteins (the protein used in the flu vaccine). Table 6 demonstrates examples of one important tyrosine kinase prediction sites (Lck) in influenza HA proteins and HIV envelope proteins. The same approach used for HCV and GBV-C will be used to engineer more potent and long-lived vaccines against influenza and other viral pathogens (including HIV). HIV is notoriously non-immunogenic, and the inventors propose that the HIV envelope glycoprotein 160 (gp160; comprised of two proteins—gp41 and gp120) inhibit T cell activation, contributing to poor immunogenicity and memory responses. By substituting critical amino acid residue(s) necessary for function, the inventors propose that this will enhance titer and memory to influenza HA. Since conserved neutralizing antibodies are present in the stalk region of influenza, we propose that this advance may lead to a universal influenza vaccine. At the least, prolonged memory against flu would obviate the need for changing the antigen for each strain each year, as most years, one or more of the trivalent strains does not change. Interestingly, influenza B has significantly fewer predicted Lck substrate motifs Table 6. A long noted clinical observation is that protection against influenza B following vaccination lasts considerably longer than that observed for influenza A (Cecil, 2012).

TABLE 6
Examples of Influenza and HIV Envelope
Predicted Lck Substrate Sites
(SEQ ID NOS: 3-37)

| | | | Kinase |
|---|---|---|---|
| Virus* | Envelope | Position | site |
| Influenza A | HA (H1) | 162 | AKSFYKNLI |
| | HA (H1) | 175 | KGNSYPKLS |
| | HA (H1) | 209 | QQSLYQNAD |
| | HA (H1) | 215 | NADAYVFVG |
| | HA (H1) | 366 | VDGWYGYHH |
| | HA (H1) | 463 | VKNLYEKVR |
| | HA (H1) | 501 | KNGTYDYPK |
| | HA (H1) | 528 | STRIYQILA |
| | HA (H1) | 534 | ILAIYSTVA |
| Influenza A | HA (H3) | 121 | DVPDYASLR |
| | HA (H3) | 177 | LNFKYPALN |
| | HA (H3) | 194 | FDKLYIWGV |
| | HA (H3) | 367 | MDGWYGFRH |
| Influenza A | HA (H3) #2 | 121 | DVPDYASLR |
| | HA (H3) #2 | 177 | LNFKYPALN |
| | HA (H3) #2 | 194 | FDKLYIWGV |
| | HA (H3) #2 | 367 | VDGWYGFRH |
| | HA (H3) #2 | 507 | DHDVYRDEA |
| Influenza B | HA | 315 | LHEKYGGLN |
| HIV JQ085296 | Gp160 | 38 | WVTVYYGVP |
| | Gp160 | 185 | SNDTYRLIN |
| | Gp160 | 323 | IRQAYCNLS |
| | Gp160 | 376 | GEFFYCNTT |
| | Gp160 | 396 | LNNTYTKEK |
| | Gp160 | 613 | SNKTYDTIW |
| | Gp160 | 632 | EIDNYTNII |
| | Gp160 | 647 | TNIIYSLIE |
| | Gp160 | 706 | VRQGYSPLS |
| HIV AF067158 | Gp160 | 38 | WVTVYYGVP |
| | Gp160 | 181 | SSEYYRLIN |
| | Gp160 | 306 | GQTFYATGD |
| | Gp160 | 372 | GEFFYCNTS |
| | Gp160 | 383 | FNGTYNWTE |
| | Gp160 | 623 | TNTIYRLLE |
| | Gp160 | 692 | VRQGYSPLS |

*Influenza and HIV envelope amino acid sequences were randomly chosen selected from GenBank.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,683,202
U.S. Pat. No. 4,879,236
U.S. Pat. No. 5,440,013
U.S. Pat. No. 5,446,128
U.S. Pat. No. 5,475,085
U.S. Pat. No. 5,618,914
U.S. Pat. No. 5,650,298
U.S. Pat. No. 5,670,155
U.S. Pat. No. 5,672,681
U.S. Pat. No. 5,674,976
U.S. Pat. No. 5,710,245
U.S. Pat. No. 5,840,833
U.S. Pat. No. 5,859,184
U.S. Pat. No. 5,925,565
U.S. Pat. No. 5,928,906
U.S. Pat. No. 5,929,237
U.S. Pat. No. 5,935,819
U.S. Pat. No. 6,261,569
Almendro, et al., J Immunol., 157(12):5411-21, 1996.
Alexandropoulos et al., Proc Natl Acad Sci USA, 92:3110-3114, 1995.
Angel et al., Cell, 49:729, 1987b.
Angel et al., Mol. Cell. Biol., 7:2256, 1987a.
Ausubel et al., Current Protocols in Molecular Biology, Greene Publishing Associates and Wiley Interscience, N. Y., 1994.
Baichwal and Sugden, In: Gene Transfer, Kucherlapati (Ed.), NY, Plenum Press, 117-148, 1986.
Bala, et al., Hepatology, 56:1946-1957, 2012.
Banerji et al., Cell, 27(2 Pt 1):299-308, 1981.
Banerji et al., Cell, 33(3):729-740, 1983.
Bao et al., Nuc Acids Res. 35:W280W284, 2007.
Barany and Merrifield, In: The Peptides, Gross and Meienhofer (Eds.), Academic Press, NY, 1-284, 1979.
Berkhout et al., Cell, 59:273-282, 1989.
Bhattarai et al., J Immunol., 189:2211-2216, 2012a.
Bhattarai et al., J Infect Dis., 206:1469-1472, 2012b.
Bhattarai et al., J Immunol., 189: 2211-2216, 2013.
Bhattarai and Stapleton, Trends Microbiol 20:124-130, 2012.
Blanar et al., EMBO J., 8:1139, 1989.
Boshart et al., Cell, 41:521, 1985.
Bosze et al., EMBO J., 5(7):1615-1623, 1986.
Bowen and Walker, Nature 436:946-952, 2005.
Braddock et al., Cell, 58:269, 1989.
Brydak and Machala, Drugs 60: 35-53, 2000.
Campo et al., Nature, 303:77, 1983.
Capaldi et al., Biochem. Biophys. Res. Comm., 74(2):425-433, 1977.
Carbonelli et al., FEMS Microbiol. Lett., 177(1):75-82, 1999.
Cecil, J. Hepatol. In Press, 2012.
Celander et al., J. Virology, 62:1314, 1988.
Cerny and Chisari, Hepatology 30:595-601, 1999.
Chandler et al., Cell, 33:489, 1983.
Chandler et al., Proc Natl. Acad. Sci. USA, 94(8):3569-601, 1997.
Chang et al., Mol. Cell. Biol., 9:2153, 1989.
Chatterjee et al., Proc Natl. Acad Sci. U.S.A., 86:9114, 1989.
Chen and Okayama, Mol. Cell Biol., 7:2745-2752, 1987.
Cheng et al., Immunol. Rev., 241:63-76, 2011.
Chivero, et al., 19th International Symposium on HCV and Related Viruses, Venice, Italy, 2012.
Cocea, Biotechniques, 23(5):814-6, 1997.
Cohen et al., J. Cell. Physiol., 5:75, 1987.
Costa et al., Mol. Cell. Biol., 8:81, 1988.
Cripe et al., EMBO J., 6:3745, 1987.
Dandolo et al., J. Virology, 47:55-64, 1983.
Davis and van der Merwe, Trends Immunol 32:1-5, 2011.
Deeks, et al., Blood, 104:942-947, 2004.
Deschamps et al., Science, 230:1174-1177, 1985.
De Villiers et al., Nature, 312(5991):242-246, 1984.
Dhanasekaran et al., Hepatic Medicine: Evidence and Res. 4:19-37, 2012.
Doganiuc, et al., Alchohol Clin. Exp. Res. 27:1023-1031, 2003.
Dreux, et al., Cell Host Microbe, 12:558-570, 2012.
Eckels, et al., Hum. Immunol. 60:187-199, 1999.
Edbrooke et al., Mol. Cell. Biol., 9:1908, 1989.
Edlund et al., Science, 230:912-916, 1985.
Fabbri, et al., Proc Natl Acad Sci USA, 109:E2110-2116, 2012.
Farci et al., Proc. Natl. Acad. Sci. USA 93:15394-15399, 1996.
Fechheimer et al., Proc. Nat'l Acad. Sci. USA, 84:8463-8467, 1987.
Fournillier, et al., J. Virol. 75:12088-12097, 2001.
Fraley et al., Proc. Natl. Acad. Sci. USA, 76:3348-3352, 1979.
Fujita et al., Cell, 49:357, 1987.
George et al., J. Infect. Dis., 193:451-454, 2006.
George et al., Virology, 316:191-201, 2003.
Ghendon, Acta Virol. 34: 295-304, 1990.
Ghosh and Bachhawat, In: Liver diseases, targeted diagnosis and therapy using specific receptors and ligands, (Wu G, Wu C ed.), NY, Marcel Dekker, 87-104, 1991.
Gilles et al., Cell, 33:717, 1983.
Gloss et al., EMBO J., 6:3735, 1987.
Godbout et al., Mol. Cell. Biol., 8:1169, 1988.
Goodbourn et al., Cell, 45:601, 1986.
Gopal, Mol. Cell Biol., 5:1188-1190, 1985.
Gossen et al., Proc. Natl. Acad. Sci. USA, 89:5547-5551, 1992.
Gossen et al., Science, 268:1766-69, 1995.
Graham and Van Der Eb, Virology, 52:456-467, 1973.
Greene et al., Immunology Today, 10:272, 1989
Grossman, et al., Nat Med, 12:289-295, 2006.
Hahn, Curr. Opin. Immunol. 15:443-449, 2003.
Harland and Weintraub, J. Cell Biol., 101:1094-1099, 1985.
Hazenberg et al., AIDS, 17:1881-1888, 2003.
Heaton et al., Cancer Res., 53:2597-2602, 1993.
Hensel et al., Lymphokine Res., 8:347, 1989.
Hirochika et al., J. Virol., 61:2599, 1987.
Holbrook et al., Virology, 157:211, 1987.
Huang et al., Cell, 27:245, 1981.
Hug et al., Mol. Cell. Biol., 8:3065, 1988.
Hunt, et al., J Infect Dis, 197:126-133, 2008.
Hunt, et al., J Infect Dis, 187:1534-1543, 2003.
Hwang et al., Mol. Cell. Biol., 10:585, 1990.
Imagawa et al., Cell, 51:251, 1987.
Imler et al., Mol. Cell. Biol, 7:2558, 1987.
Isaguliants et al., Vaccine 22:1656-1665, 2004.
Jakobovits et al., Mol. Cell. Biol., 8:2555, 1988.
Jaynes et al., Mol. Cell. Biol., 8:62, 1988.
Johannesson et al. J. Med. Chem., 42(22):4524-4537, 1999.
Johannesson et al., J. Med. Chem., 42:601-608, 1999.
Johnson et al., In: Biotechnology And Pharmacy, Pezzuto et al., (Eds.), Chapman and Hall, NY, 1993.
Johnson et al., Mol. Cell. Biol., 9:3393, 1989.
Kaneda et al., Science, 243:375-378, 1989.
Kanto, et al., J. Immunol. 162:5584-5591, 1999.
Karin et al., Mol. Cell. Biol., 7:606, 1987.
Katinka et al., Cell, 20:393, 1980.
Kawamoto et al., Mol. Cell. Biol., 8:267, 1988.
Kiledjian et al., Mol. Cell. Biol., 8:145, 1988.
Kittleson et al., J. Clin. Invest. 106:1239-1249, 2000.
Klamut et al., Mol. Cell. Biol., 10:193, 1990.
Kobayashi et al., J. Gastroenterol. 33:500-507, 1998.
Koch et al., Mol. Cell. Biol., 9:303, 1989.
Kraus et al., FEBS Lett., 428(3):165-70, 1998.
Krey, et al., PLoS Pathog, 6:e1000762, 2010.
Kriegler and Botchan, In: Eukaryotic Viral Vectors, Y. Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Krishnada, et al., Int. Immunol. 22:491-502, 2010.
Kuhl et al., Cell, 50:1057, 1987.
Kunz et al., Nucl. Acids Res., 17:1121, 1989.
Kyte and Doolittle, J. Mol. Biol., 157:105-132, 1982.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lauer and Walker, N Engl. J Med. 345:41-52, 2001.
Lechner et al., Eur. J. Immunol. 30:2479-2487, 2000.
Lee et al., DNA Cell Biol., 16(11):1267-1275, 1997.
Lee et al., Nature, 294:228, 1981.
Lee et al., Nucleic Acids Res., 12:4191-206, 1984.
Levenson et al., Hum. Gene Ther., 9(8):1233-6, 1998.
Levinson et al., Nature, 295:79, 1982.
Lin et al., Mol. Cell. Biol., 10:850, 1990.
Luria et al., EMBO J., 6:3307, 1987.
Lusky et al., Mol. Cell. Biol. 3:1108, 1983.
Maidana-Giret et al., AIDS, 23:2277-2287, 2009.
Masciopinto et al., Eur J Immunol, 34:2834-2842, 2004.
McLinden et al., J. Virol., 80:12131-12140, 2006.
McNeall et al., Gene, 76:81, 1989.
Meckes and Raab-Traub, J Virol, 85:12844-12854, 2011.
Merrifield, Science, 232(4748):341-347, 1986.
Miksicek et al., Cell, 46:203, 1986.
Mohr et al., J. Immunol., 185:4496-4505, 2010.
Moreau et al., Nucl. Acids Res., 9:6047, 1981.
Muesing et al., Cell, 48:691, 1987.
Nattermann et al., AIDS, 17:1457-1462, 2003.
Ng et al., Nuc. Acids Res., 17:601, 1989.
Nicolas and Rubenstein, In: Vectors: A survey of molecular cloning vectors and their uses, Rodriguez and Denhardt (eds.), Stoneham: Butterworth, 493-513, 1988.
Nicolau and Sene, Biochim. Biophys. Acta, 721:185-190, 1982.
Nomoto et al., Gene, 236(2):259-271, 1999.
Nunnari et al., Ann. Intern. Med., 139:26-30, 2003.
Obenauer, et al., Nucleic Acids Res, 31:3635-3641, 2003.
Ondek et al., EMBO J., 6:1017, 1987.
Palmiter et al., Nature, 300:611, 1982.
Pech et al., Mol. Cell. Biol., 9:396, 1989.
Pelletier and Sonenberg, Nature, 334(6180):320-325, 1988.
Pereira et al., Kidney Int. 53:1374-1381, 1998.
Pichlmair, et al., Nature, 487:486-490, 2012.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Nat'l Acad. Sci. USA, 81:7161-7165, 1984.
Potter, et al., J. Virol. 86:12923-12932, 2012.
Quinn et al., Mol. Cell. Biol., 9:4713, 1989.
Redondo et al., Science, 247:1225, 1990.
Remington's Pharmaceutical Sciences, 15th Ed., 33:624-652, 1990.
Remington's Pharmaceutical Sciences, 18th Ed. Mack Printing Company, 1389-1404, 1990,
Rey et al., Eur. J. Clin. Microbiol. Infect. Dis., 19:721-724, 2000.
Ridgeway, In: Vectors: A survey of molecular cloning vectors and their uses, Rodriguez and Denhardt (Eds.), Stoneham:Butterworth, 467-492, 1988.
Ridpath, Biologicals 41:14-19, 2013.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Rydze et al., Antivir Ther 17:1271-1279, 2012.
Sakai et al., Genes and Dev., 2:1144, 1988.
Saksela et al., Embo J 14:484-491, 1995.
Satake et al., J. Virology, 62:970, 1988.
Schaffner et al., J Mol. Biol., 201:81, 1988.
Schotsaert, et al., Expert Rev Vaccines 11:949-962, 2012.
Schwarze-Zander et al., Antivir. Ther., 15:745-752, 2010.
Searle et al., Mol. Cell. Biol., 5:1480, 1985.
Seidman et al., Influenza Other Respir. Viruses 6:52-62, 2012
Semmo et al., Hepatol. 41:1019-1028, 2005.
Serti et al., Cell. Mol. Life Sci. 68:505-522, 2011.
Sherman et al., Mol. Cell. Biol., 9:50, 1989.
Shimizu, J. Virol., 73:8411-8414, 1999.
Singh, et al., J Immunol, 189:777-785, 2012.
Soguero et al., J Virol. 76:9345-9354, 2002.
Soto, et al., J Immunol, 184:4185-4195, 2010.
Spalholz et al., Cell, 42:183, 1985.
Stapleton et al., AIDS, 23:605-610, 2009.
Stapleton et al., J Gen. Virol., 92:233-246, 2011.
Stapleton et al., AIDS 23:605-610, 2009.
Stapleton et al., J Gen Virol 92:233-246, 2011.
Stapleton et al., PLoS One 7:e50563, 2012.
Stapleton et al., AIDS 27: 1829-1832, 2103.
Stewart and Young, In: Solid Phase Peptide Synthesis, 2^{nd} Ed., Pierce Chemical Co., 1984.
Stuart et al., Nature, 317:828, 1985.
Takebe et al., Mol. Cell. Biol., 8:466, 1988.
Tam et al., J. Am. Chem. Soc., 105:6442, 1983.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (ed.), NY: Plenum Press, 149-188, 1986.
Thiesen et al., J. Virology, 62:614, 1988.
Tillmann et al., N. Engl. J. Med., 345:715-724, 2001.
Tomova, et al., Anticancer Res. 29:5241-5244, 2009.
Treisman, Cell, 42:889, 1985.
Tronche et al., Mol. Biol. Med., 7:173, 1990.
Tur-Kaspa et al., Mol. Cell Biol., 6:716-718, 1986.
Tyndell et al., Nuc. Acids. Res., 9:6231, 1981.
Vasseur et al., Proc Natl. Acad. Sci. USA, 77:1068, 1980.
Vinikoor, et al., J Acquir Immune Defic Syndr, 2013
Vita et al., Biopolymers, 47:93-100, 1998.
Weber et al., Cell, 36:983, 1984.
Weinberger et al. Mol. Cell. Biol., 8:988, 1984.
Weisshoff et al., Eur. J. Biochem., 259(3):776-788, 1999.
Williams et al., N. Engl. J. Med., 350:981-990, 2004.
Wrammert and Ahmed, Biol. Chem. 389:537-539, 2008.
Wu and Wu, Biochem., 27:887-892, 1988.
Wu and Wu, J. Biol. Chem., 262:4429-4432, 1987.
Wu et al., Biochem. Biophys. Res. Commun., 233(1):221-6, 1997.
Wu et al., J. Med. Virol., 52:83-85. 1997.
Xiang et al., J Virol 72:2738-2744, 1998.
Xiang et al., J. Viral Hepat., 6:S16-S22, 1999.
Xiang et al., J. Virol. 74:9125-9133, 2000.
Xiang et al., N Engl. J. Med., 345:707-714, 2001.
Xiang, et al., Lancet 363:2040-2046, 2004.
Xiang et al., Proc. Natl. Acad. Sci USA, 103:15570-15575, 2006.
Xiang et al., PLoS One 3: e2580, 1-10, 2008.
Xiang et al., Virology 430:53-62, 2012.
Xue et al., Mol Cell Proteomics 7:1598-1608, 2008.
Xu et al., J. Theor. Biol. 344: 78-87, 2014.
Yang et al., Proc. Natl. Acad. Sci USA, 87:9568-9572, 1990.
Yutzey et al. Mol. Cell. Biol., 9:1397, 1989.
Zhang et al., HIV Med 7:173-180, 2006.
Zhuang et al., Embo J 31:3513-3523, 2012.

1. A method of inhibiting immune cell activation comprising administering to a mammalian subject in need thereof an RNA virus envelope peptide or polypeptide comprising an immunomodulatory domain.

2. The method of claim 1, wherein said peptide or polypeptide comprises about 16, 17, 18, 19, 20, 21, 22, 23, 24, 25, 30, 35, 40, 45, 50, 75, 100, 150, 175, 200, 219, 250 consecutive residues of a native envelope polypeptide or immunomodulatory domain.

3. The method of claim 1, wherein the peptide or polypeptide comprises HCV E2 sequences.

4. The method of claim 3, wherein the peptide or polypeptide comprises non-HCV E2 sequences.

5. The method of claim 1, wherein the immune cell is a T cell or a B cell.

6. The method of claim 5, wherein the T cell is a helper T cell suppressor T cell, or a killer T cell.

7. The method of claim 1, wherein said subject is a human.

8. The method of claim 1, wherein administering comprises intravenous, intraarterial, oral, subcutaneous, topical or intraperitoneal administration.

9. The method of claim 1, further comprising administering a second anti-inflammatory agent.

10-15. (canceled)

16. The method of claim 1, wherein said RNA virus envelope peptide or polypeptide is not GBV-C E2.

17. The method of claim 1, wherein said peptide or polypeptide is administered at 0.1-500 mg/kg/d.

18-19. (canceled)

20. The method of claim 1, wherein the peptide or polypeptide is derived from Hepatitis C Virus E2, Human Immunodeficiency Virus envelope gp120/160, Yellow Fever Virus envelope protein, Bovine Viral Diarrhea Virus envelope protein, Classical Swine Fever Virus envelope protein, influenza envelope protein, Dengue Virus envelope protein, West Nile Virus envelope protein, and Japanese Encephalitis Virus envelope protein.

21. A composition comprising a peptide or polypeptide comprising a peptide segment as shown in FIG. 19 or 21, formulated with a pharmaceutically acceptable carrier buffer or diluent.

22-25. (canceled)

26. A method of inducing an immune response in an mammalian subject comprising administering to said subject with an RNA virus envelope protein wherein said envelope protein comprises one or more modified kinase sites.

27. The method of claim 26, wherein said modified kinase site comprises a deleted kinase site or a mutated kinase site.

28-30. (canceled)

31. The method of claim 26, wherein said envelope protein is comprised in a subunit vaccine comprising other viral components but lacking intact virions, or wherein said enveloped protein is comprised in a killed whole virion, or wherein said enveloped protein is comprised in a live attenuated virus.

32-33. (canceled)

34. The method of claim 26, wherein said envelope protein is administered with a second envelope protein from a distinct serotype or strain of said virus.

35-38. (canceled)

39. The method of claim 26, wherein said kinase site is an Lck site or Fyn site.

40-42. (canceled)

43. A vaccine comprising an RNA virus envelope protein having a modification in a peptide segment shown in Table 5 or FIG. 19 or 21.

44-50. (canceled)
